(19) United States
(12) Patent Application Publication
(10) Pub. No.: US 2002/0122798 A1
(43) Pub. Date: Sep. 5, 2002

(54) Compounds for targeting

(76) Inventor: Robert Young, London (GB)

Correspondence Address: JOHN S. PRATT, KILPATRICK STOCKTON LLP, 1100 PEACHTREE, SUITE 2800, ATLANTA, GA, 30309, US

(21) Appl. No.: 09/825,012

(22) Filed: Apr. 3, 2001

Related U.S. Application Data
(60) Provisional application No. 60/237,159, filed on Oct. 2, 2000.

(30) Foreign Application Priority Data
Apr. 3, 2000 (GB) GB 0008049.9

(51) Int. Cl. A61K 39/395; C07K 16/46

(52) U.S. Cl. 424/141.1; 424/178.1; 530/388.15

(57) ABSTRACT

A compound comprising a target cell-specific portion and a cytotoxic portion characterized in that the target cell-specific portion comprises an humanized monoclonal antibody having specificity for polymorphic epithelial mucin (PEM), or an antigen binding fragment thereof, and the cytotoxic portion has endonucleolytic activity. Preferably, the target cell-specific portion comprises an humanized HMFG-1 antibody or an antigen binding fragment thereof. Advantageously, the cytotoxic portion is at least the catalytically active portion of a DNA endonuclease, e.g. a human DNA endonuclease I.

The invention further provides nucleic acids encoding the compounds of the invention, and the use of such compounds in medicine, e.g. in the treatment of cancer.

The present invention relates to cytotoxic compounds that have a high avidity for, and can be targeted to, selected cells. Specifically, the invention provides compounds comprising a cytotoxic portion having DNA endonucleolytic activity and a target-cell specific portion having specificity for human polymorphic epithelial mucin (PEM).

### BACKGROUND

The cell-specific targeting of compounds that are directly, or indirectly, cytotoxic has been proposed as a way to combat diseases such as cancer. Bagshawe and his co-workers have disclosed (Bagshawe (1987) Br. J. Cancer 56, 531; Bagshawe et al (1988) Br. J. Cancer 58, 700; WO 88/07378) conjugated compounds comprising an antibody or part thereof and an enzyme, the antibody being specific to tumour cell antigens and the enzyme acting to convert an innocuous pro-drug into a cytotoxic compound. The cytotoxic compounds were alkylating agents, e.g. a benzoic acid mustard released from para-N-bis(2-chloroethyl)aminobenzoyl glutamic acid by the action of Pseudomonas sp. CPG2 enzyme.

An alternative system using different pro-drugs has been disclosed (WO 91/11201) by Epenetos and co-workers. The cytotoxic compounds were cyanogenic monosaccharides or disaccharides, such as the plant compound amygdalin, which release cyanide upon the action of a β-glucosidase and hydroxynitrile lyase.

In a further alternative system, the use of antibody-enzyme conjugates containing the enzyme alkaline phosphatase in conjunction with the pro-drug etoposide 4′-phosphate or 7-(2′-aminoethyl phosphate)mitomycin or a combination thereof have been disclosed (EP 0 302 473; Senter et al (1988) Proc. Natl. Acad. Sci. USA 85, 4842).

Rybak and co-workers have disclosed (Rybak et al (1991) J. Biol. Chem. 266, 21202; WO 91/16069) the cytotoxic potential of a monomeric pancreatic ribonuclease when injected directly into Xenopus oocytes and the cytotoxic potential of monomeric RNase coupled to human transferrin or antibodies directed against the transferrin receptor. The monomeric RNase hybrid proteins were cytotoxic to human erythroleukaemia cells in vitro.

Other approaches are the in vivo application of streptavidin conjugated antibodies followed, after an appropriate period, by radioactive biotin (Hnatowich et al (1988) J. Nucl. Med. 29, 1428-1434), or injection of a biotinylated mAb followed by radioactive streptavidin (Paganelli et al (1990) Int. J. Cancer 45, 1184-1189). A pilot radioimmunolocalisation study in non-small cell lung carcinomas was conducted with encouraging results (Kalofonos et al (1990) J. Nucl. Med. 31, 1791-1796).

Apart from these examples, it is rather more common to see biotinylated antibodies and streptavidin-enzyme conjugates, which are used in enzyme-linked immunosorbent assays.

These previous systems have used relatively large antibody-enzyme, antibody-streptavidin or antibody-biotin conjugates and may comprise portions of non-mammalian origin which are highly immunoreactive.

We have now devised improved compounds for targeting cells to be destroyed.

### SUMMARY OF INVENTION

A first aspect of the invention provides a compound comprising a target cell-specific portion and a cytotoxic portion characterised in that the target cell-specific portion comprises an humanised monoclonal antibody having specificity for polymorphic epithelial mucin (PEM), or an antigen binding fragment thereof, and the cytotoxic portion has endonucleolytic activity.

By “target cell specific” portion we mean the portion of the compound which comprises one or more binding sites which recognise and bind to polymorphic epithelial mucin (PEM) on the target cell. Upon contact with the target cell, the target cell specific portion is preferably internalised along with the cytotoxic portion. Such internalisation results in the cytotoxic portion being delivered to the cell cytosol, where it has access to the cell's nucleic acid molecules.

The target cell-specific portion of the compounds of the invention comprises an humanised monoclonal antibody having specificity for polymorphic epithelial mucin (PEM), or an antigen binding fragment thereof.

Polymorphic epithelial mucin, or PEM, is a component of the human milk fat globule. PEM is expressed by cells in several body tissues and is also found in urine. Significantly, PEM is known to be expressed in epithelial cancer cells, notably in ovarian, gastric, colorectal and pancreatic cancer cells.

Monoclonal antibodies which will bind to PEM are already known, but in any case, with today's techniques in relation to monoclonal antibody technology, antibodies can be prepared to most antigens. The antigen-specific portion may be a whole antibody, a part of an antibody (for example a Fab or F(ab′)₂ fragment), a synthetic antibody fragment (for example a single chain Fv fragment [ScFv]), or a peptide/peptidomimetic or similar. Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982) and Antibody Engineering, A Practical Approach, McCafferty, J. et al, ed. (IRL Pres, 1996).

By ‘humanised monoclonal antibody’ we include monoclonal antibodies having at least one chain wherein the framework regions are predominantly derived from a first, acceptor monoclonal antibody of human origin and at least one complementarity-determining region (CDR) is derived from a second, donor monoclonal antibody having specificity for PEM. The donor monoclonal antibody may be of human or non-human origin, for example it may be a murine monoclonal antibody.

Preferably, both chains of the humanised monoclonal antibody comprise CDRs grafted from a donor monoclonal antibody having specificity for PEM.

Advantageously, the CDR-grafted (i.e. humanised) chain comprises two or all three CDRs derived from a donor antibody having specificity for PEM.

Conveniently, the humanised monoclonal antibody comprises only human framework residues and CDRs from a donor antibody having specificity for PEM.

However, it will be appreciated by those skilled in the art that in order to maintain and optimise the specificity of the humanised antibody it may be necessary to alter one or more residues in the framework regions such that they correspond to equivalent residues in the donor antibody.

Conveniently, the framework regions of the humanised antibody are derived from an human IgG monoclonal antibody.

Methods of making humanised monoclonal antibodies are well-known in the art, for example see Jones et al. (1986) Nature 321:522-525, Riechmann et al. (1988) Nature 332:323-327, Verhoeyen et al. (1988) Science 239:1534-1536 and EP 239 400 (to Winter).

In a preferred embodiment of the first aspect of the invention, the target cell-specific portion comprises an humanised HMFG-1 monoclonal antibody or an antigen binding fragment thereof. HMFG antibodies are raised against human milk fat globule (HMFG), in a delipidated state (see Taylor-Papadimiriou et al., 1981, Int. J. Cancer 28:17-21 and Gendler et al., 1988, J. Biol. Chem. 236:1282-12823). HMFG-1 monoclonal antibodies bind to a particular component of HMFG, namely polymorphic epithelial mucin (PEM). Binding is thought to involve the amino acid sequence APDTR within the twenty amino acid tandem repeats of the muc-1 gene product.

Exemplary humanised HMFG-1 antibodies are disclosed in WO 92/04380.

Advantageously, the target cell-specific portion is an humanised HMFG-1 monoclonal antibody.

In a preferred embodiment of the first aspect of the invention, the target cell-specific portion comprises a fragment of an humanised monoclonal antibody having specificity for polymorphic epithelial mucin (PEM), said fragment retaining the antigen binding properties of the parent antibody.

The variable heavy (V_{H}) and variable light (V_{L}) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sci. USA 81, 6851-6855).

That antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); disulphide-linked Fv molecules (Young et al., 1995, FEBS Lett. 377:135-139); single-chain Fv (ScFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dabs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

By “ScFv molecules” we mean molecules wherein the V_{H} and V_{L} partner domains are linked via a flexible oligopeptide.

Chimaeric antibodies are discussed by Neuberger et al (1988, 8th International Biotechnology Symposium Part 2, 792-799).

The advantages of using antibody fragments, rather than whole antibodies, are several-fold. The smaller size of the fragments allows for rapid clearance, and may lead to improved tumour to non-tumour ratios. Fab, Fv, ScFv, disulphide Fv and dAb antibody fragments can all be expressed in and secreted from bacteria, such as E. coli, or eukaryotic expression systems such as Yeast or mammalian systems, thus allowing the facile production of large amounts of the said fragments.

Whole antibodies, and F(ab′)₂ fragments are “bivalent”. By “bivalent” we mean that the said antibodies and F(ab′)₂ fragments have two antigen combining sites. In contrast, Fab, Fv, ScFv, disulphide Fv and dAb fragments are monovalent, having only one antigen combining site.

Preferably, the target cell-specific portion of the compounds of the invention comprises an antigen binding fragment of the humanised antibody selected from the group consisting of Fab-like molecules, such as Fab and F(ab′)₂, Fv molecules, disulphide-linked Fv molecules, ScFv molecules and single domain antibodies (dAbs).

More preferably, the target cell-specific portion comprises a Fab molecule or a F(ab′)₂ molecule.

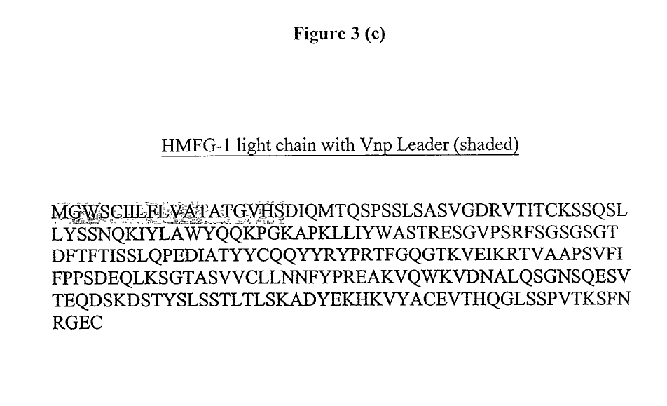

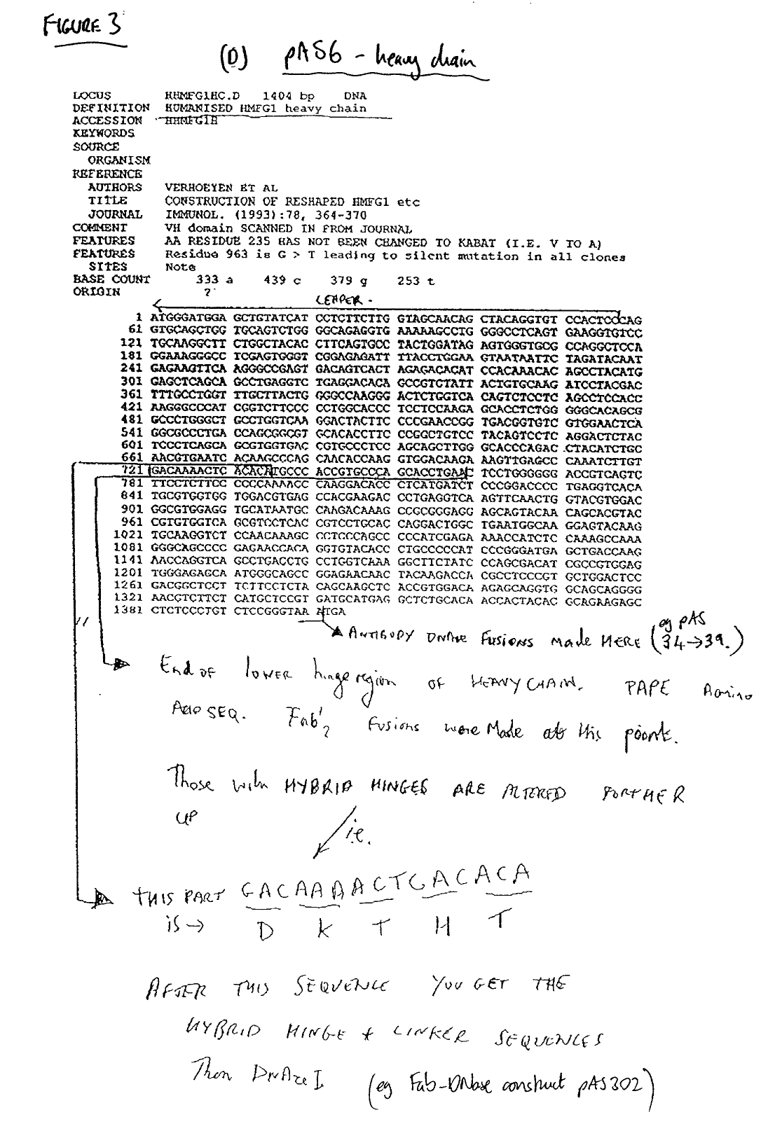

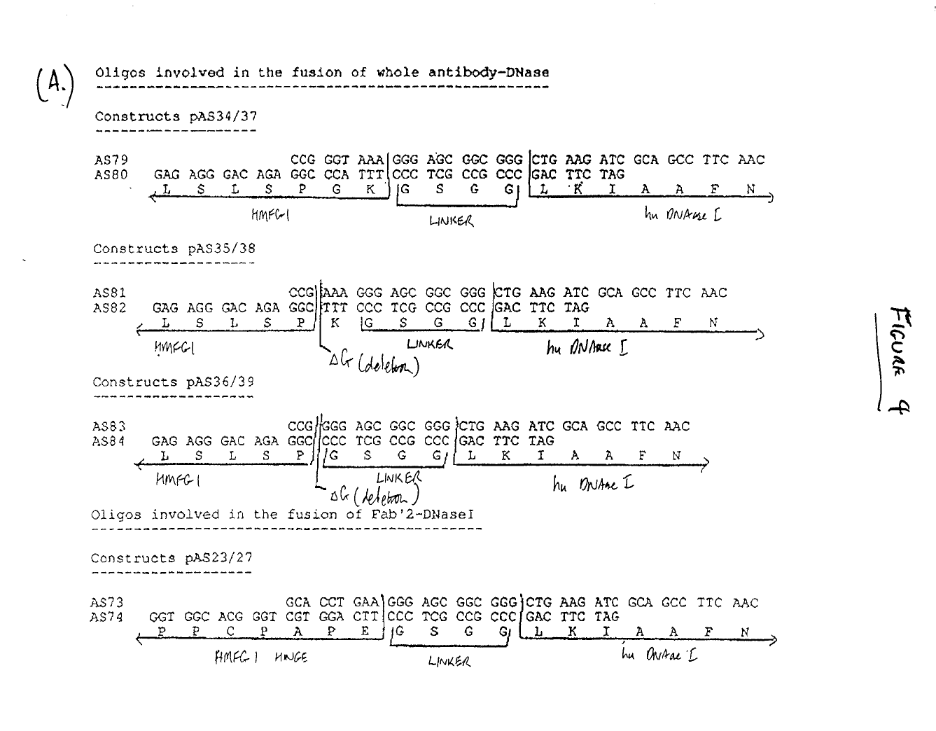

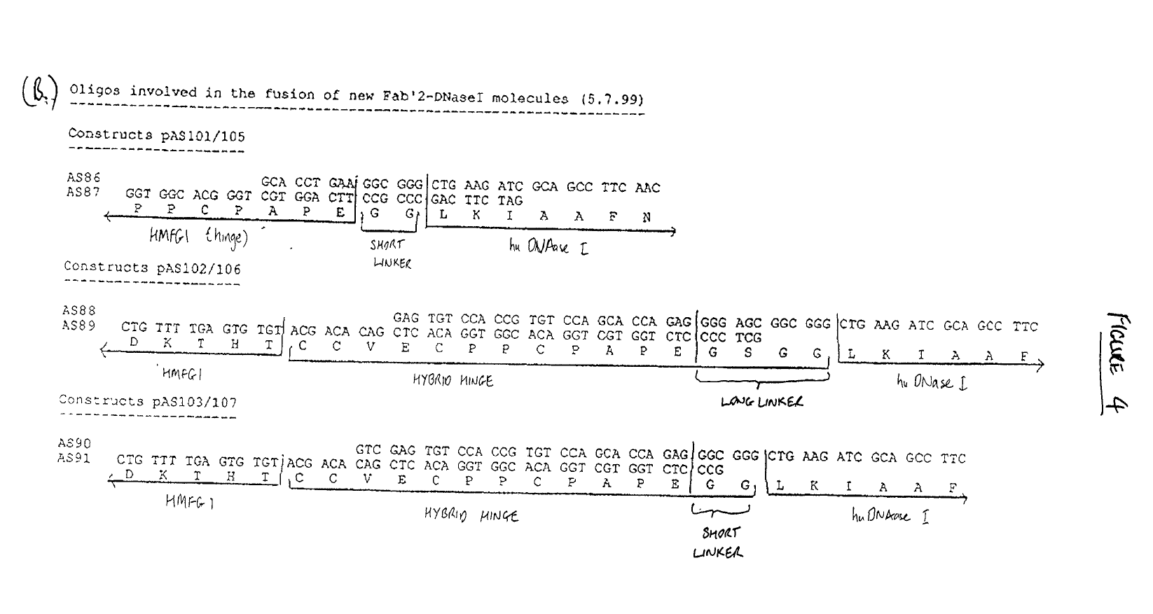

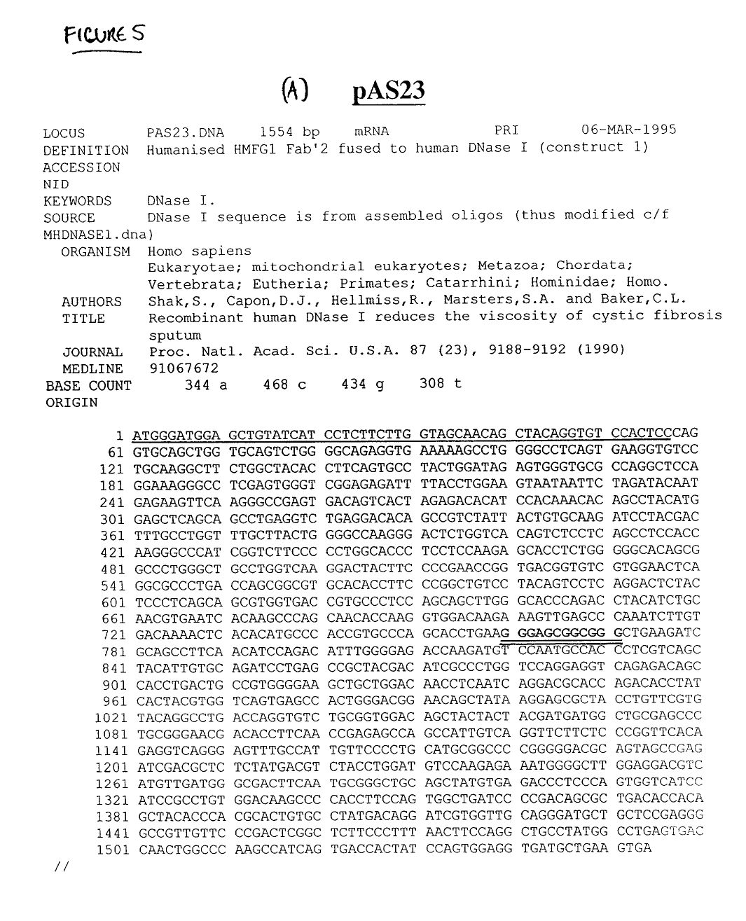

Yet more preferably, the target cell-specific portion comprises an amino acid sequence encoded by at last part of one or both of the nucleotide sequences of FIG. 3(a) and (d).

Most preferably, the target cell-specific portion comprises an amino acid sequence encoded by the nucleotide sequence of FIG. 3(a) and an amino acid sequence encoded by the nucleotide sequence of FIG. 3(d).

Preferably, the target cell-specific portion recognises the target cell with high avidity.

By “high avidity” we mean that the target cell-specific portion recognises the target cell with a binding constant of at least K_{d}=10⁻⁶M, preferably at least K_{d}=10⁻⁹M, suitably K_{d}=10⁻¹⁰M, more suitably K_{d}=10⁻¹¹M, yet more suitably still K_{d}=10⁻¹²M, and more preferably K_{d}=10⁻¹⁵M or even K_{d}=10⁻¹⁸M.

Preferably, the target cell-specific portion comprises an antigen binding fragment of an humanised HMFG-1 monoclonal antibody, e.g. an Fab or F(ab′)₂ fragment thereof, wherein a hinge region contains a mutation (i.e. wherein the hinge is a variant or hybrid of a naturally occurring hinge). More preferably, the variant hinge comprises the amino acid sequence CCVECPPCPAPE.

By ‘cytotoxic portion’ we mean a portion having endonucleolytic activity which is toxic to the cell if it is to reach, and preferably enter said cell.

In a preferred embodiment of the first aspect of the invention, the cytotoxic portion has DNA endonucleolytic activity.

Advantageously, the cytotoxic portion is at least the catalytically active portion of a DNA endonuclease.

Examples of known DNA endonucleases include bovine DNase I (see Worrall and Conolly, 1990, J. Biol. Chem. 265:21889-21895). Human pancreatic DNase I has also been cloned (see Shak et al., 1990, Proc.

Natl. Acad. Sci. USA 87:9188-9192 and Hubbard et al., 1992, New Eng. J. Med. 326:812-815).

Preferably, the endonuclease is a mammalian deoxyribonuclease I.

More preferably, the endonuclease is a human deoxyribonuclease I.

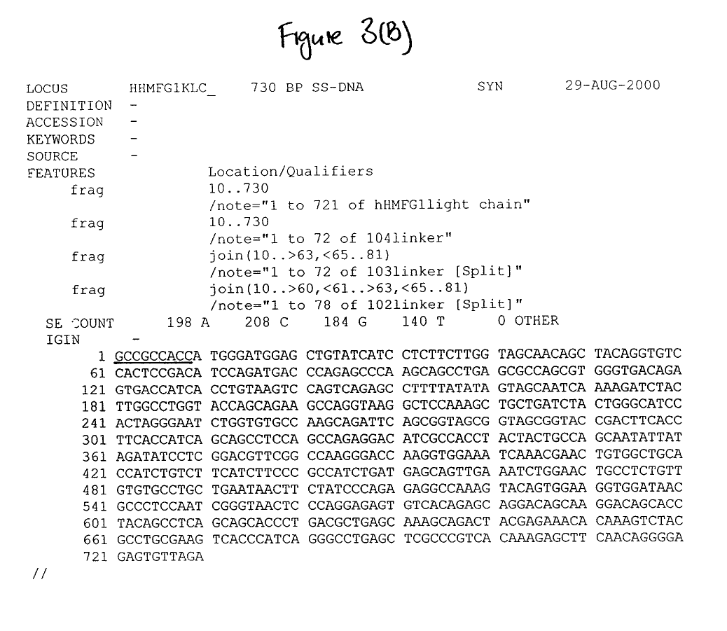

Most preferably, the cytotoxic portion comprises the amino acid sequence shown in FIG. 2(a) or 2(b).

Preferably, the cytotoxic portion of the compound of the invention is capable of oligomerisation, e.g. dimerisation. Attachment of the target-cell specific portion to a cytotoxic portion capable of oligomerisation provides a method for increasing the number of binding sites to the target cell. For example, if the target cell-specific portion is joined to a portion capable of forming a dimer then the number of target cell-specific binding sites is two; if the target cell-specific portion is joined to a portion capable of forming a tetramer then the number of target cell-specific binding sites is four. The number of target cell-specific binding sites is greater than one and the compounds may therefore have a greater avidity for the target cell than do compounds which only have one target cell-specific binding site.

It is preferable for the cytotoxic portion of the compound of the invention capable of oligomerisation to contain no interchain disulphide bonds nor intrachain disulphide bonds; to be well characterised; to be non-toxic; to be stable; to be amenable to preparation in a form suitable for pre-clinical or clinical use or be in pre-clinical or clinical use; and for the subunit monomers to have a high affinity for each other, that is they contain one or more subunit binding sites.

Advantageously, the cytotoxic portion is of mammalian, preferably human, origin. The use of the said mammalian proteins as the cytotoxic portion of the compound of the invention is advantageous since such compounds are less likely to give rise to undesirable immune reactions.

It will be appreciated by those skilled in the art that the cytotoxic portion may be a variant of a naturally occurring endonuclease.

By “a variant” we include cytotoxic portions comprising of a naturally occurring endonuclease wherein there have been amino acid insertions, deletions or substitutions, either conservative or non-conservative, such that the changes do not substantially reduce the endonuclease activity of the variant compared to that of the naturally occurring endonuclease. For example, the variant may have increased activity compared to the naturally occurring endonuclease.

Such variants may be made using methods of protein engineering and site-directed mutagenesis commonly known in the art (for example, see Sambrook et al., 1989, Molecular cloning: A Laboratory Manual, 2^{nd} edition, Cold Spring Harbor Laboratory Press, NY, USA).

In an alternative embodiment, the endonuclease is a restriction endonuclease, such as a microbial type II restriction endonuclease. Exemplary type II restriction endonucleases include BamHI, HindIII, MspI, Sau3AI, HinfI, NotI and EcoRI.

In another preferred embodiment of the first aspect of the invention, a nuclear localization signal is incorporated into the compound.

Preferably, the nuclear localization signal (NLS) comprises a nuclear localization signal from the SV40 large T antigen (Kalderon et al., 1984, Cell 39:499-509), and specifically the amino acid sequence PKKKRKV.

Inclusion of a nuclear localization signal encourages the compound of the invention to gain access to the chromosomal DNA during the periods of the cell cycle when the nuclear membrane is intact, since the nuclear pores are permeable to large molecules incorporating said nuclear localization signal.

In a further preferred embodiment of the first aspect of the invention, the target cell-specific portion and the cytotoxic portion are fused to create a fusion compound.

By “fusion compound” we include a compound comprising one or more functionally distinct portions, wherein the distinct portions are contained within a single polypeptide chain produced by recombinant DNA techniques. For example, the compound may comprise a whole antibody wherein the heavy chain is fused to human DNase I. Alternatively, the compound may comprise an Fab or F(ab′)₂ fragment of an antibody wherein the truncated heavy chain (i.e. the Fd chain) is fused to human DNase I.

Preferably, the target-cell specific and the cytotoxic portion of the fusion compound of the invention separated by a linker sequence, for example to allow greater flexibility of the portions relative to one another.

More preferably, the linker sequence comprises a GG dipeptide.

Most preferably the linker sequence is or comprises GG or GSGG.

Alternatively, the target-cell specific and the cytotoxic portion of the compound of the invention are separate moieties linked together by any of the conventional ways of cross-linking polypeptides, such as those generally described in O'Sullivan et al Anal. Biochem. (1979) 100, 100-108. For example, the antibody portion may be enriched with thiol groups and the enzyme portion reacted with a bifunctional agent capable of reacting with those thiol groups, for example the N-hydroxysuccinimide ester of iodoacetic acid (NHIA) or N-succinimidyl-3-(2-pyridyldithio)propionate (SPDP). Amide and thioether bonds, for example achieved with m-maleimidobenzoyl-N-hydroxysuccinimide ester, are generally more stable in vivo than disulphide bonds.

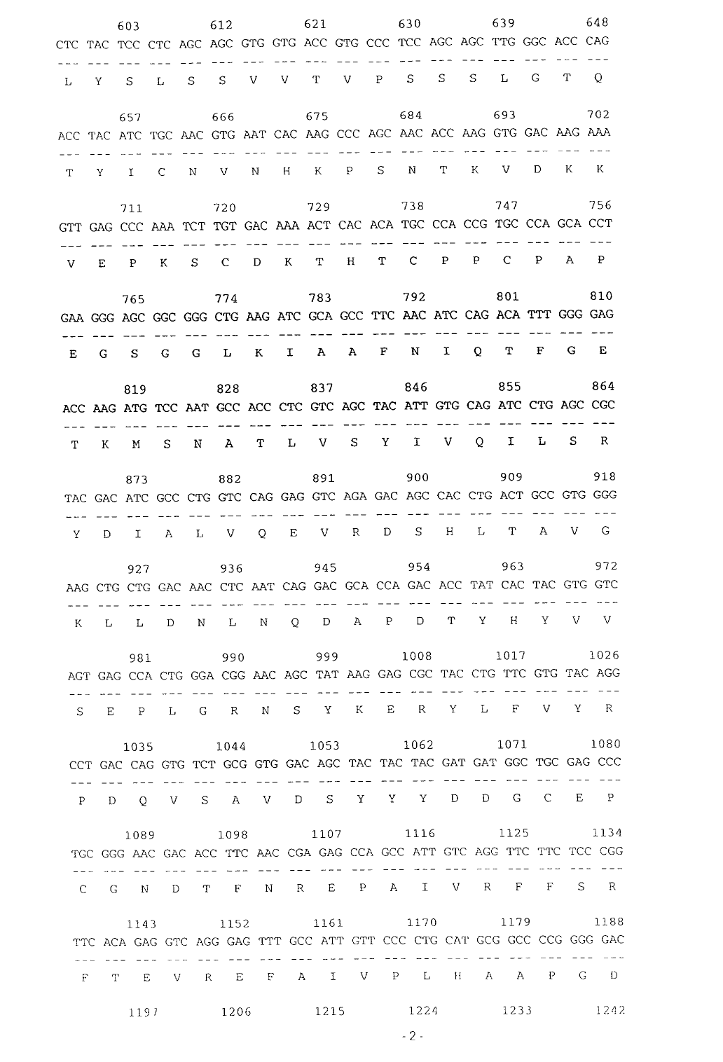

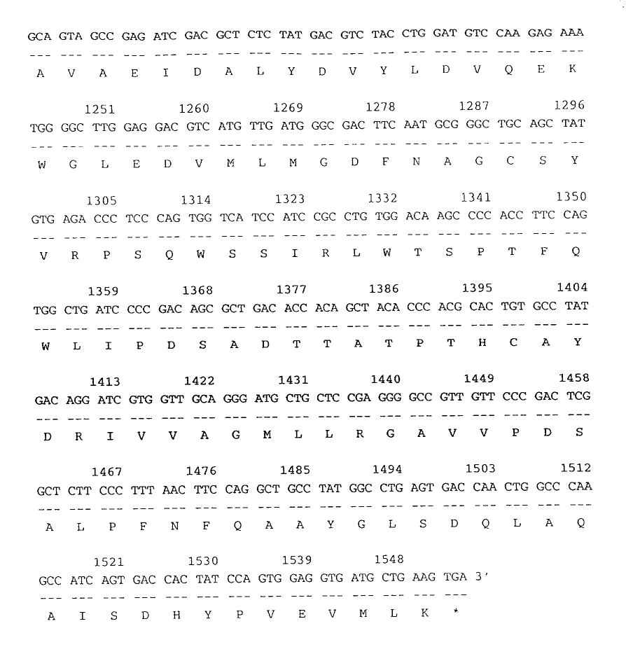

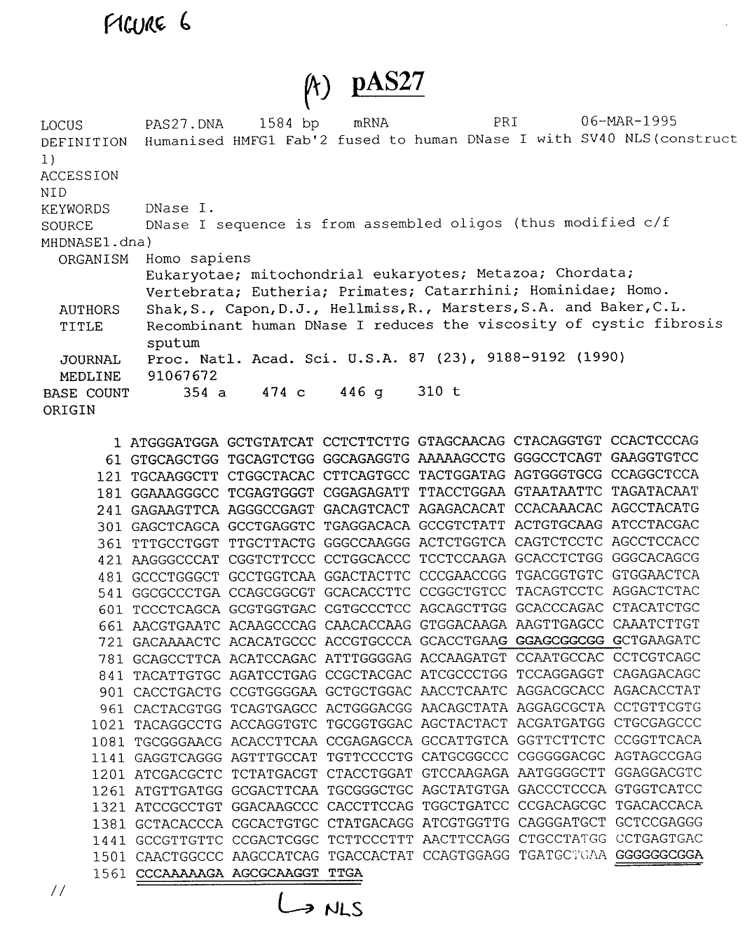

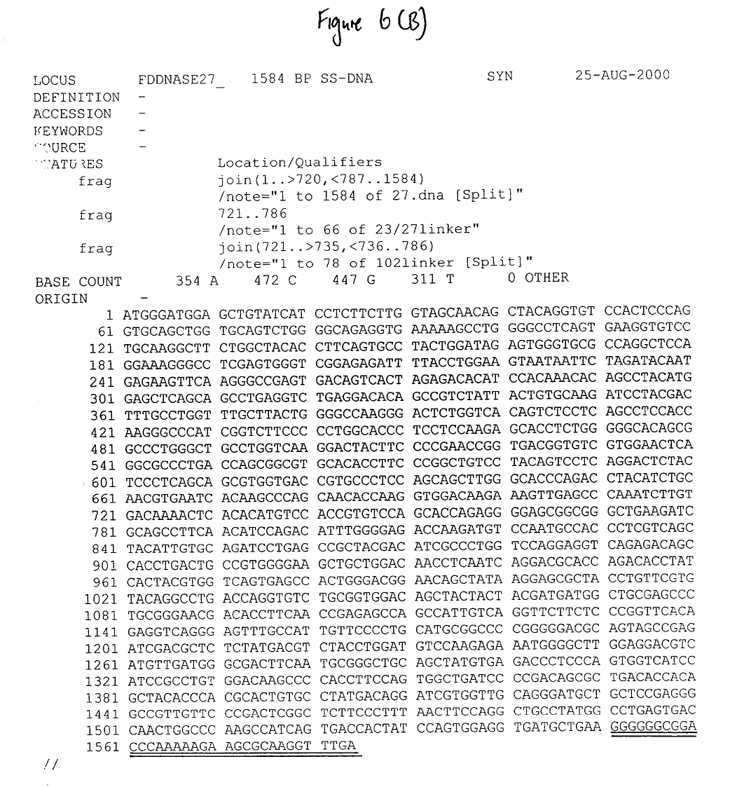

In a preferred embodiment of the first aspect of the invention, the compound comprises all or part of the amino acid sequence as shown in FIG. 3(c) (i.e. an HMFG-1 light chain) together with all or part of an amino acid sequence selected from the group consisting of amino acid sequences as shown in FIGS. 5(d), 6(d), 7(b), 8(b), 9(b), 10(b), 11(b), 12(b), 13(d), 14(d), 15(d), 16(c), 17(d), 18(d) and 19(d) (i.e. an HMFG-1 heavy or Fd chain/DNase fusion).

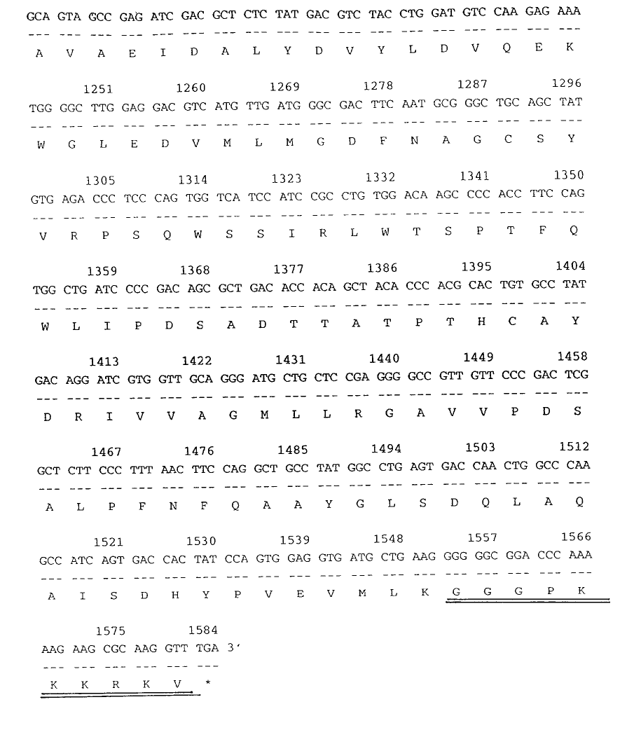

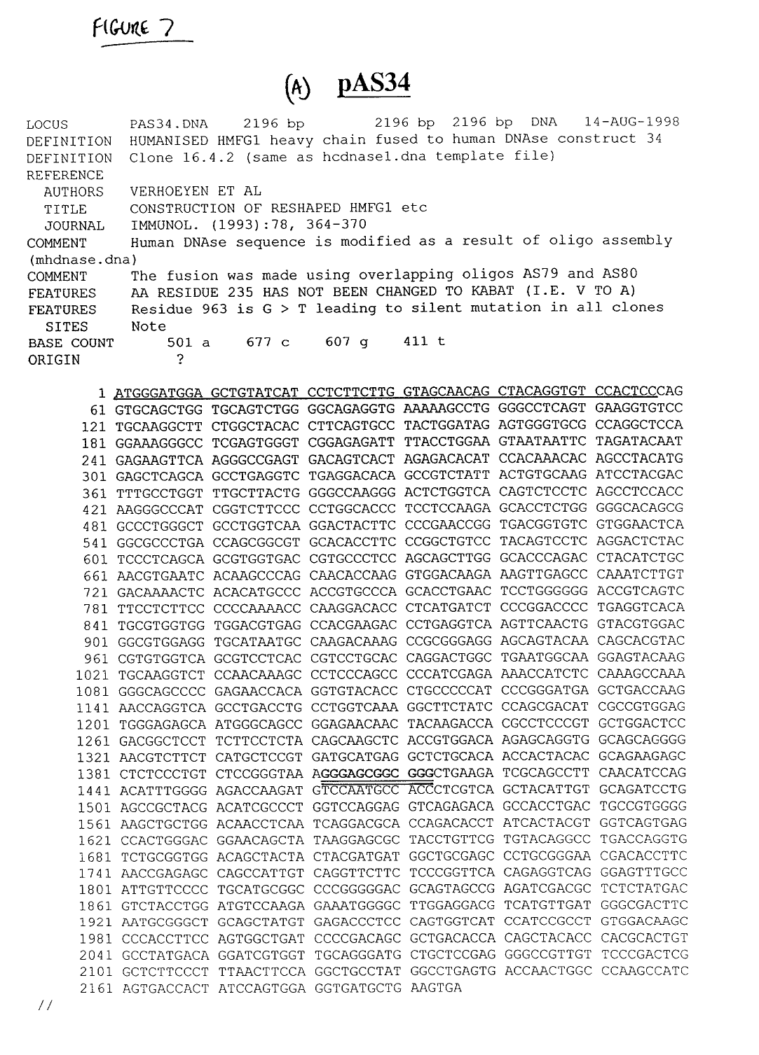

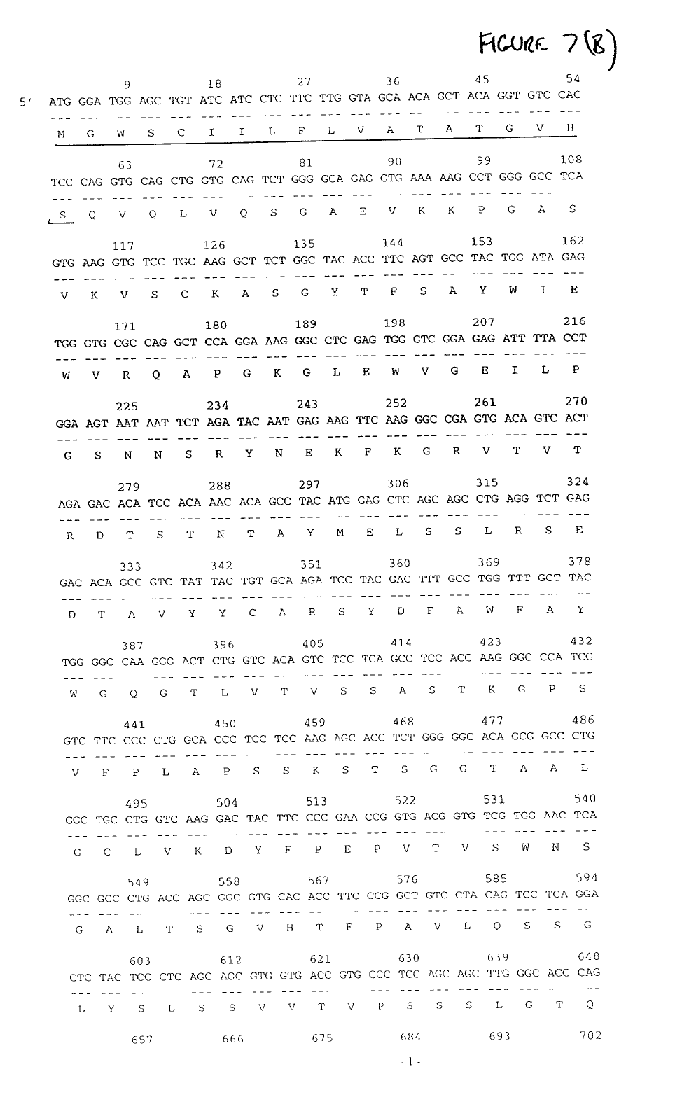

Advantageously, the compound is a whole HMFG-1 antibody/human DNase I fusion compound comprising an amino acid sequence as shown in FIG. 3(c) and an amino acid sequence as shown in FIG. 7(b). Preferably, the compound is a tetrameric compound comprising two HMFG-1 light chains and two HMFG- 1 heavy chain/DNase I fusions.

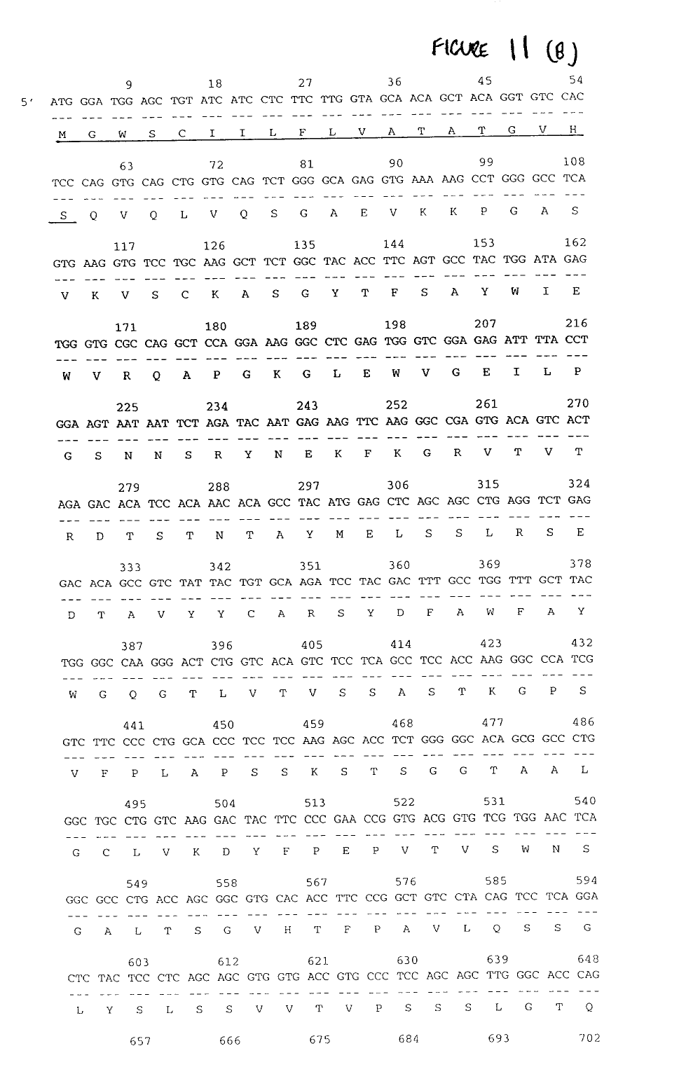

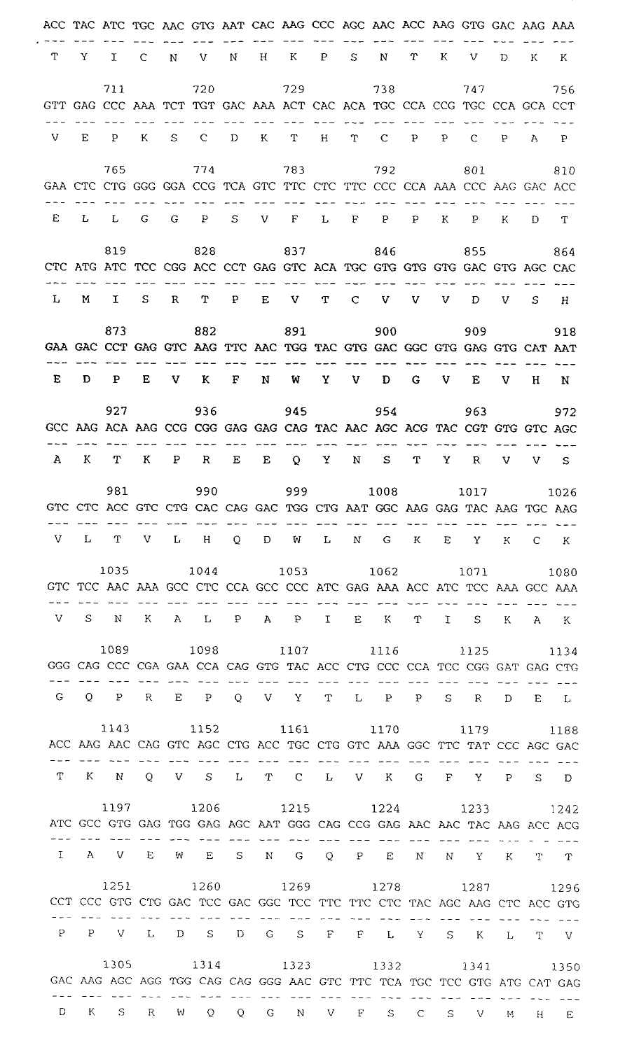

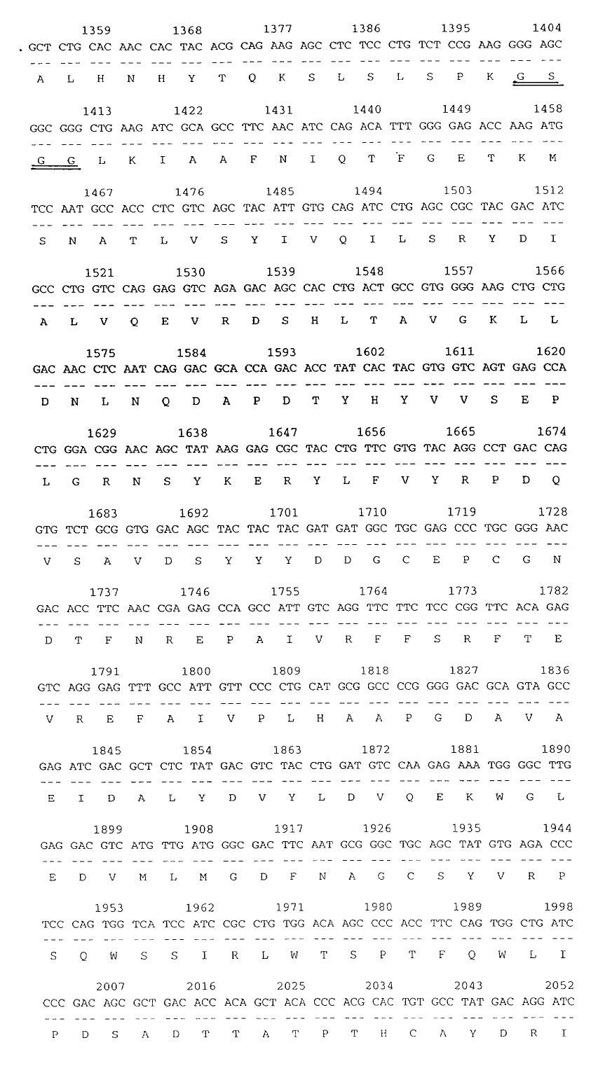

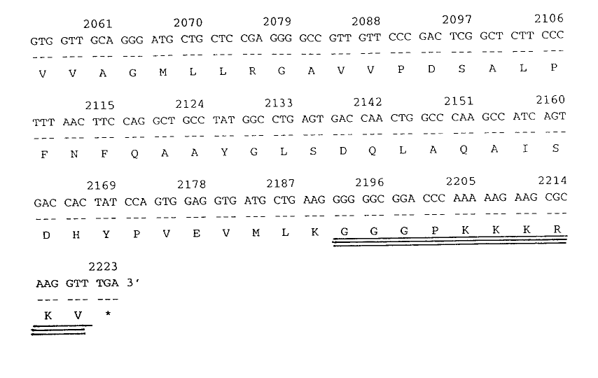

Conveniently, the compound comprises an amino acid sequence as shown in FIG. 3(c) and an amino acid sequence as shown in FIG. 14(d).

Preferably, the compound comprises one of the pairs of amino acid sequences defined above wherein the leader sequence of each amino acid (the first 19 amino acids of the sequences shown in each figure) is removed. It will be appreciated by persons skilled in the art that the compounds of the invention may also comprise variants of such amino acid sequences.

Suitably, the compound is a tetrameric compound comprising two HMFG-1 light chains and two HMFG-1 Fd chain/DNase I fusions. More preferably, the compound is a dimeric compound comprising one HMFG-1 light chain and one HMFG-1 Fd chain/DNase I fusion.

A second aspect of the invention provides a nucleic acid molecule encoding a compound according to the first aspect of the invention, or a target cell-specific portion or cytotoxic portion thereof.

By “nucleic acid molecule” we include DNA, cDNA and MRNA molecules.

In a preferred embodiment of the second aspect of the invention, the nucleic acid molecule comprises all or part of the nucleotide sequence as shown in FIG. 3(a or b) (i.e. encoding an HMFG-1 light chain) together with all or part of a nucleotide sequence selected from the group consisting of nucleotide sequences as shown in FIGS. 5(a, b and c), 6(a, b and c), 7(a), 8(a), 9(a), 10(a), 11(a), 12(a), 13(a, b and c), 14(a, b and c), 15(a, b and c), 16(a and b), 17(a, b and c), 18(a, b and c) and 19(a, b and c) (i.e. encoding an HMFG-1 heavy or Fd chain/DNase fusion).

Advantageously, the nucleic acid molecule comprising a nucleotide sequence as shown in FIG. 3(b) and a nucleotide sequence as shown in FIG. 7(a).

Conveniently, the compound comprises a nucleotide sequence as shown in FIG. 3(b) and a nucleotide sequence as shown in FIG. 14(c).

Alternatively, the nucleic acid molecule comprises nucleotide sequences that are degenerate sequences of those nucleotide sequences identified above (i.e. which encode the same amino acid sequence).

A further aspect of the present invention provides a method of making a compound according to the first aspect of the invention, said method comprising expressing one or more nucleic acid molecules according to the second aspect of the invention in a host cell and isolating the compound therefrom.

It is preferable that the two portions of the compound of the invention are produced as a fusion compound by recombinant DNA techniques, whereby a length of DNA comprises respective regions encoding the two portions of the compound of the invention either adjacent one another or separated by a region encoding a linker peptide which does not destroy the desired properties of the compound. The benefits in making the compound of the invention using recombinant DNA techniques are several fold. Firstly, it enables a high degree of precision with which the two portions of the compound can be joined together. Secondly, the construction of compounds which are “hetero-oligomeric” can be controlled by the expression of the different recombinant DNA molecules encoding each of the different type of subunit of the “hetero-oligomer” in the same host cell.

By “hetero-oligomer” we mean those compounds in which two or more different cell-specific portions are joined to either the same or to different subunits which are capable of oligomerisation. The expression, in the same host cell of two compounds, of A and B, each with different target cell specific portions but with a common second portion capable of oligomerisation will result in a mixed population of compounds. For example, if the common second portion is capable of dimerisation, three potential compounds will be produced: A₂, AB and B₂, in a ratio of 1:2:1, respectively.

The separation of the desired compound with each of the different cell specific portions, that is AB, can be achieved by two step affinity chromatography.

Application of the mixture of compounds to an affinity column specific for A will result in the binding of A₂ and AB. These compounds are eluted from this first column, and then applied to an affinity column specific for B. This will result in AB, but not A₂, being bound to the column. Finally, the desired product AB, can be eluted.

Of course, the order in which the affinity columns are used is not important.

The same principle of separating those compounds with two or more different binding sites can be applied to the purification of the desired compounds from mixtures of other hetero-oligomers.

Conceivably, the two portions of the compound may overlap wholly or partly.

Preferably, the compound is a multimeric compound such as a whole antibody/DNase fusion comprising two light chains and two heavy chains (H₂L₂), a F(ab′)₂ fusion comprising two light chains and two truncated heavy chains (Fd₂L₂), or a Fab fusion comprising one light chain and one truncated heavy chain (FdL).

The nucleic acid may be expressed in a suitable host to produce a polypeptide comprising the compound of the invention. Thus, the nucleic acid encoding the compound of the invention or a portion thereof may be used in accordance with known techniques, appropriately modified in view of the teachings contained herein, to construct an expression vector, which is then used to transform an appropriate host cell for the expression and production of the polypeptide of the invention. Such techniques include those disclosed in U.S. Pat. No. 4,440,859 issued Apr. 3, 1984 to Rutter et al, U.S. Pat. No. 4,530,901 issued Jul. 23, 1985 to Weissman, U.S. Pat. No. 4,582,800 issued Apr. 15, 1986 to Crowl, U.S. Pat. No. 4,677,063 issued Jun. 30, 1987 to Mark et al, U.S. Pat. No. 4,678,751 issued Jul. 7, 1987 to Goeddel, U.S. Pat. No. 4,704,362 issued Nov. 3, 1987 to Itakura et al, U.S. Pat. No. 4,710,463 issued Dec. 1, 1987 to Murray, U.S. Pat. No. 4,757,006 issued Jul. 12, 1988 to Toole, Jr. et al, U.S. Pat. No. 4,766,075 issued Aug. 23, 1988 to Goeddel et al and U.S. Pat. No. 4,810,648 issued Mar. 7, 1989 to Stalker, all of which are incorporated herein by reference.

Where the compound of the invention is multimeric, the constituent chains may be encoded by a single nucleic acid molecule or separate nucleic acid molecule (expressed in a common host cell or in different host cells and assembled in vitro).

The nucleic acid encoding the compound of the invention or a portion thereof may be joined to a wide variety of other nucleic acid sequences for introduction into an appropriate host. The companion nucleic acid will depend upon the nature of the host, the manner of the introduction of the nucleic acid into the host, and whether episomal maintenance or integration is desired.

It will be appreciated that in order to prevent expression of the cytotoxic portion of the compound of the invention from killing the host cells in which it is expressed, it may be necessary to link the nucleic acid of the second aspect of the invention to a signal sequence capable of directing secretion of the expressed compound (or portion) out of the host cell. Signal sequences will be selected according to the type of host cell used. Exemplary signal sequences include the ompA signal sequence (for example, see Takahara et al., 1985, J. Biol. Chem. 260(5):2670-2674).

Generally, the nucleic acid is inserted into an expression vector, such as a plasmid, in proper orientation and correct reading frame for expression. If necessary, the nucleic acid may be linked to the appropriate transcriptional and translational regulatory control nucleotide sequences recognised by the desired host, although such controls are generally available in the expression vector. For example, the nucleic acid molecule encoding a compound of the invention may be linked to or comprise a Kozak consensus ribosome binding sequence (such as GCCGCCACC) to enhance translation.

The vector is then introduced into the host through standard techniques. Generally, not all of the hosts will be transformed by the vector. Therefore, it will be necessary to select for transformed host cells. One selection technique involves incorporating into the expression vector a nucleic acid sequence, with any necessary control elements, that codes for a selectable trait in the transformed cell, such as antibiotic resistance. Alternatively, the gene for such selectable trait can be on another vector, which is used to co-transform the desired host cell.

Host cells that have been transformed by the recombinant nucleic acid of the invention are then cultured for a sufficient time and under appropriate conditions known to those skilled in the art in view of the teachings disclosed herein to permit the expression of the polypeptide, which can then be recovered.

Many expression systems are known, including bacteria (for example E. coli and Bacillus subtilis), yeasts (for example Saccharomyces cerevisiae and Pichia pastoris), filamentous fungi (for example Aspergillus), plant cells, animal cells (for example COS-1, COS-7, CHO, NIH 3T3, NSO and BHK cells) and insect cells (for example Drosophila, SF9 cells).

Those vectors that include a replicon such as a procaryotic replicon can also include an appropriate promoter such as a procaryotic promoter capable of directing the expression (transcription and translation) of the genes in a bacterial host cell, such as E. coli, transformed therewith.

A promoter is an expression control element formed by a DNA sequence that permits binding of RNA polymerase and transcription to occur. Promoter sequences compatible with exemplary bacterial hosts are typically provided in plasmid vectors containing convenient restriction sites for insertion of a DNA segment of the present invention.

Typical procaryotic vector plasmids are pUC18, pUC19, pBR322 and pBR329 (available from Biorad Laboratories, Richmond, Calif., USA), pTrc99A and pKK223-3 (available from Pharmacia Piscataway, N.J., USA) and the pET system (T7 promoter, Novagen Ltd).

A typical mammalian cell vector plasmid is pSV_{L} available from Pharmacia, Piscataway, N.J., USA. This vector uses the SV40 late promoter to drive expression of cloned genes, the highest level of expression being found in T antigen-producing cells, such as COS-1 cells.

An example of an inducible mammalian expression vector is pMSG, also available from Pharmacia. This vector uses the glucocorticoid-inducible promoter of the mouse mammary tumour virus long terminal repeat to drive expression of the cloned gene.

Useful yeast plasmid vectors are pRS403-406 and pRS413-416 and are generally available from Stratagene Cloning Systems, La Jolla, Calif. 92037, USA. Plasmids pRS403, pRS404, pRS405 and pRS406 are Yeast Integrating plasmids (YIps) and incorporate the yeast selectable markers his3, trp1, leu2 and ura3. Plasmids pRS413-416 are Yeast Centromere plasmids (YCps).

Further useful vectors for transformation of yeast cells, such as Pichia, include the 2μ plasmid pYX243 (available from R and D Systems Limited) and the integrating vector pPICZ series (available from Invitrogen).

A variety of methods have been developed to operatively link DNA to vectors via complementary cohesive termini. For instance, complementary homopolymer tracts can be added to the DNA segment to be inserted to the vector DNA. The vector and DNA segment are then joined by hydrogen bonding between the complementary homopolymeric tails to form recombinant DNA molecules.

Synthetic linkers containing one or more restriction sites provide an alternative method of joining the DNA segment to vectors. The DNA segment, generated by endonuclease restriction digestion as described earlier, is treated with bacteriophage T4 DNA polymerase or E. coli DNA polymerase I, enzymes that remove protruding, 3′-single-stranded termini with their 3′-5′-exonucleolytic activities, and fill in recessed 3′-ends with their polymerizing activities.

The combination of these activities therefore generates blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the products of the reaction are DNA segments carrying polymeric linker sequences at their ends. These DNA segments are then cleaved with the appropriate restriction enzyme and ligated to an expression vector that has been cleaved with an enzyme that produces termini compatible with those of the DNA segment.

Synthetic linkers containing a variety of restriction endonuclease sites are commercially available from a number of sources including International Biotechnologies Inc, New Haven, Conn., USA.

A desirable way to modify the nucleic acid encoding the compound of the invention or a portion thereof is to use the polymerase chain reaction as disclosed by Saiki et al (1988) Science 239, 487-491.

In this method the nucleic acid to be enzymatically amplified is flanked by two specific oligonucleotide primers which themselves become incorporated into the amplified nucleic acid. The said specific primers may contain restriction endonuclease recognition sites which can be used for cloning into expression vectors using methods known in the art.

Exemplary genera of yeast contemplated to be useful in the practice of the present invention are Pichia, Saccharomyces, Kluyveromyces, Candida, Torulopsis, Hansenula, Schizosaccharomyces, Citeromyces, Pachysolen, Debaromyces, Metschunikowia, Rhodosporidium, Leucosporidium, Botryoascus, Sporidiobolus, Endomycopsis, and the like. Preferred genera are those selected from the group consisting of Pichia, Saccharomyces, Kluyveromyces, Yarrowia and Hansenula. Examples of Saccharomyces are Saccharomyces cerevisiae, Saccharomyces italicus and Saccharomyces rouxii. Examples of Kluyveromyces are Kluyveromyces fragilis and Kluyveromyces lactis. Examples of Hansenula are Hansenula polymorpha, Hansenula anomala and Hansenula capsulata. Yarrowia lipolytica is an example of a suitable Yarrowia species.

Methods for the transformation of S. cerevisiae are taught generally in EP 251 744, EP 258 067 and WO 90/01063, all of which are incorporated herein by reference.

Suitable promoters for S. cerevisiae include those associated with the PGK1gene, GAL1 or GAL10 genes, CYC1, PHO5, TRP1, ADH1, ADH2, the genes for glyceraldehyde-3-phosphate dehydrogenase, hexokinase, pyruvate decarboxylase, phosphofructokinase, triose phosphate isomerase, phosphoglucose isomerase, glucokinase, α-mating factor pheromone, a-mating factor pheromone, the PRB1 promoter, the GUT2 promoter, and hybrid promoters involving hybrids of parts of 5′ regulatory regions with parts of 5′ regulatory regions of other promoters or with upstream activation sites (e.g. the promoter of EP-A-258 067).

The transcription termination signal is preferably the 3′ flanking sequence of a eukaryotic gene which contains proper signals for transcription termination and polyadenylation. Suitable 3′ flanking sequences may, for example, be those of the gene naturally linked to the expression control sequence used, i.e. may correspond to the promoter. Alternatively, they may be different in which case the termination signal of the S. cerevisiae AHD1 gene is preferred.

The present invention also relates to a host cell transformed with a polynucleotide vector construct of the present invention. The host cell can be either procaryotic or eukaryotic. Bacterial cells are preferred procaryotic host cells and typically are a strain of E. coli such as, for example, the E. coli strains DH5 available from Bethesda Research Laboratories Inc., Bethesda, Md., USA, and RR1 available from the American Type Culture Collection (ATCC) of Rockville, Md., USA (No ATCC 31343). Preferred eukaryotic host cells include yeast and mammalian cells, preferably vertebrate cells such as those from a mouse, rat, monkey or human fibroblastic cell line. Preferred eukaryotic host cells include Chinese hamster ovary (CHO) cells available from the ATCC as CCL61, NIH Swiss mouse embryo cells NIH/3T3 available from the ATCC as CRL 1658 and monkey kidney-derived COS-1 cells available from the ATCC as CRL 1650 or WSØ cells.

Transformation of appropriate cell hosts with a nucleic acid constructs of the present invention is accomplished by well known methods that typically depend on the type of vector used. With regard to transformation of procaryotic host cells, see, for example, Cohen et al, Proc. Natl. Acad. Sci. USA, 69: 2110 (1972); and Sambrook et al, Molecular Cloning, A Laboratory Manual, Cold Spring Harbor Laboratory, Cold Spring Harbor, N.Y. (1989). Transformation of yeast cells is described in Sherman et al, Methods In Yeast Genetics, A Laboratory Manual, Cold Spring Harbor, N.Y. (1986). The method of Beggs, Nature, 275: 104-109 (1978) is also useful. With regard to vertebrate cells, reagents useful in transfecting such cells, for example calcium phosphate and DEAE-dextran or liposome formulations, are available from Stratagene Cloning Systems, or Life Technologies Inc, Gaithersburg, Md. 20877, USA.

Successfully transformed cells, i.e. cells that contain a nucleic acid construct of the present invention, can be identified by well known techniques. For example, cells resulting from the introduction of an expression construct of the present invention can be grown to produce the polypeptide of the invention. Cells can be harvested and lysed and their DNA content examined for the presence of the DNA using a method such as that described by Southern, J. Mol. Biol., 98: 503 (1975) or Berent et al, Biotech., 3: 208 (1985). Alternatively, the presence of the protein in the supernatant can be detected using antibodies as described below.

In addition to directly assaying for the presence of recombinant nucleic acid, successful transformation can be confirmed by well known immunological methods when the recombinant nucleic acid is capable of directing the expression of the protein. For example, cells successfully transformed with an expression vector produce proteins displaying appropriate antigenicity. Samples of cells suspected of being transformed are harvested and assayed for the protein using suitable antibodies.

Thus, in addition to the transformed host cells themselves, the present invention also contemplates a culture of those cells, preferably a monoclonal (clonally homogeneous) culture, or a culture derived from a monoclonal culture, in a nutrient medium. Preferably, the culture also contains the protein.

Nutrient media useful for culturing transformed host cells are well known in the art and can be obtained from several commercial sources.

A third aspect of the invention provides a vector comprising a nucleic acid according to the second aspect of the invention.

A fourth aspect of the invention provides a host cell comprising a vector according to the third aspect of the invention.

Preferably, the host cell is a mammalian cell.

More preferably the host cell is NS0 or CHO.

A fifth aspect of the invention provides a pharmaceutical composition comprising a compound according to the first aspect of the invention and a pharmaceutically acceptable carrier.

The compounds and compositions of the invention are administered in any suitable way, usually parenterally, for example intravenously, intraperitoneally or, preferably (for bladder cancer), intravesically (i.e. into the bladder), in standard sterile, non-pyrogenic formulations of diluents and carriers, for example isotonic saline (when administered intravenously).

A sixth aspect of the invention provides a compound according to the first aspect of the invention for use in medicine.

The compounds and compositions of the invention may be used to treat a patient with any disease involving a dysfunction of a population of cells expressing PEM, said compounds and compositions selectively targeting and destroying said population of cells within a patient. For example, said compounds and compositions may be used in the treatment of cancer, e.g. cancer of the breast, ovaries, lung, stomach, intestines, blood etc. Thus, anti-tumour cell antigen antibodies can be used to deliver a cytotoxic portion with endonuclease activity to a tumour cell. Antibodies that are internalised upon contact with the target antigen are used, such that the cytotoxic portion enters the cytosol of the tumour cell, where it can trigger cell death.

In principle, the compounds and compositions of the invention may be used to treat any mammal, including pets such as dogs and cats and agriculturally important animals such as cows, horses, sheep and pigs.

Preferably, the patient is human.

A seventh aspect of the invention provides the use of a compound according to first aspect of the invention in the preparation of a medicament for treating a mammal having said target cells to be destroyed.

Preferably, the medicament is for treating cancer, such as ovarian cancer.

A eighth aspect of the invention provides a method of treating a mammal having target cells to be destroyed, the method comprising administering a compound according to the first aspect of the invention to said mammal.

In a preferred embodiment of the seventh and eighth aspects of the invention, the mammal is a human.

Preferably, the target cells to be destroyed are cancer cells. More preferably, the cancer cells are epithelial cancer cells, such as ovarian, gastric, colorectal and/or pancreatic cancer cells. Most preferably, the cancer cells are ovarian cancer cells.

The invention will now be described in detail with reference to the following figures and examples:

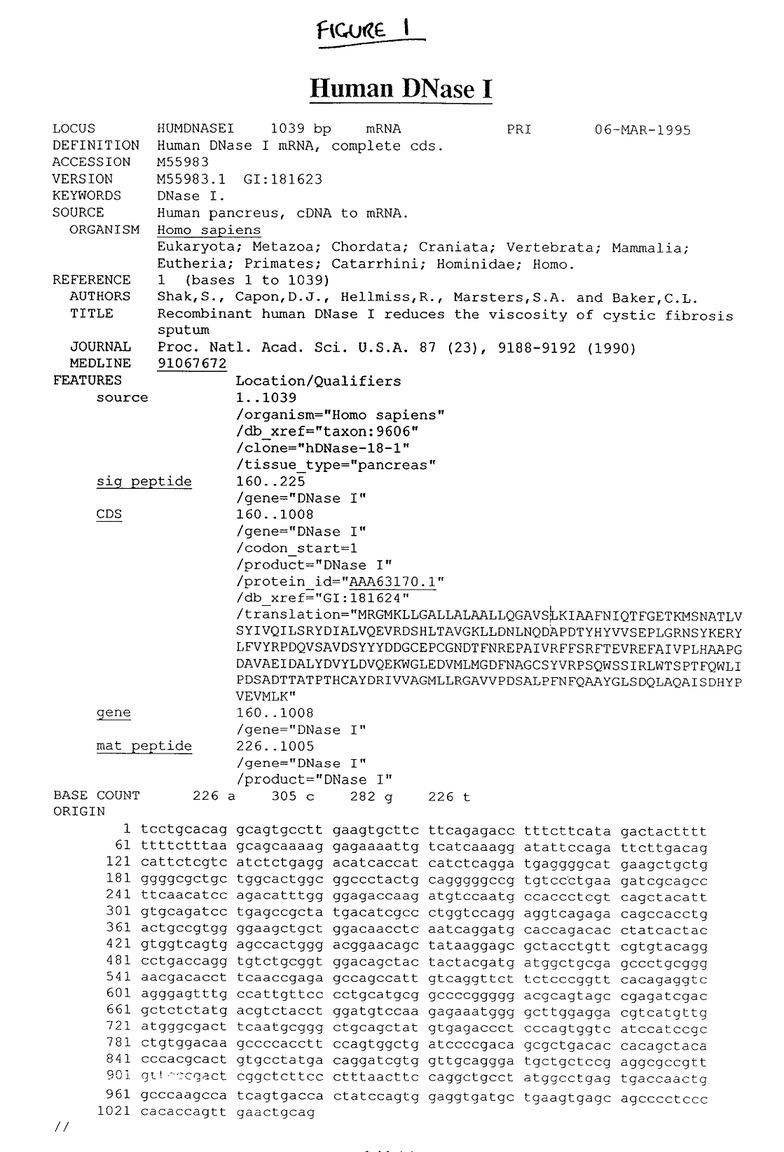

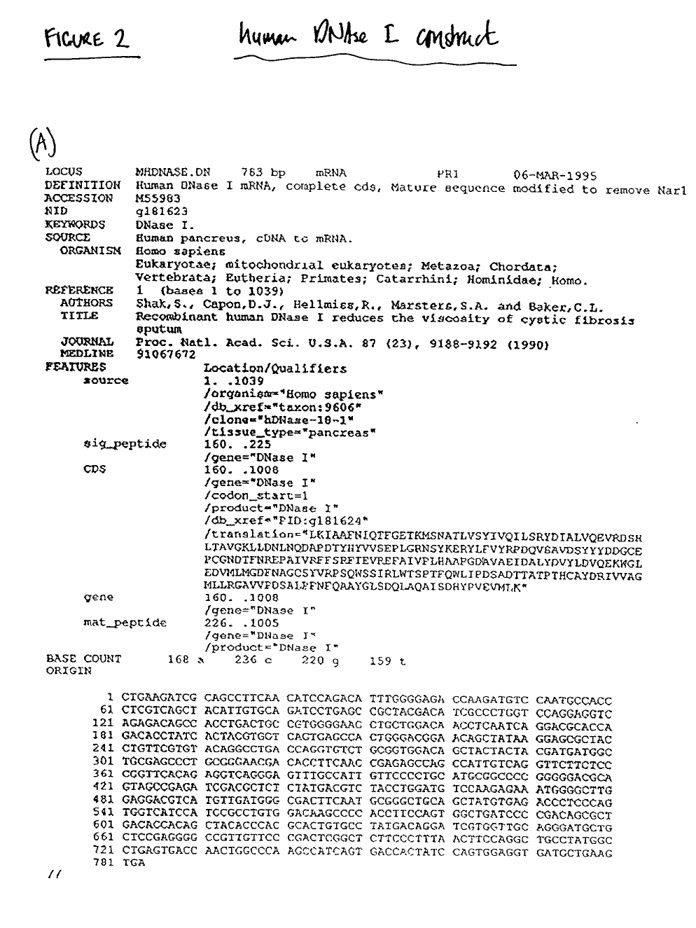

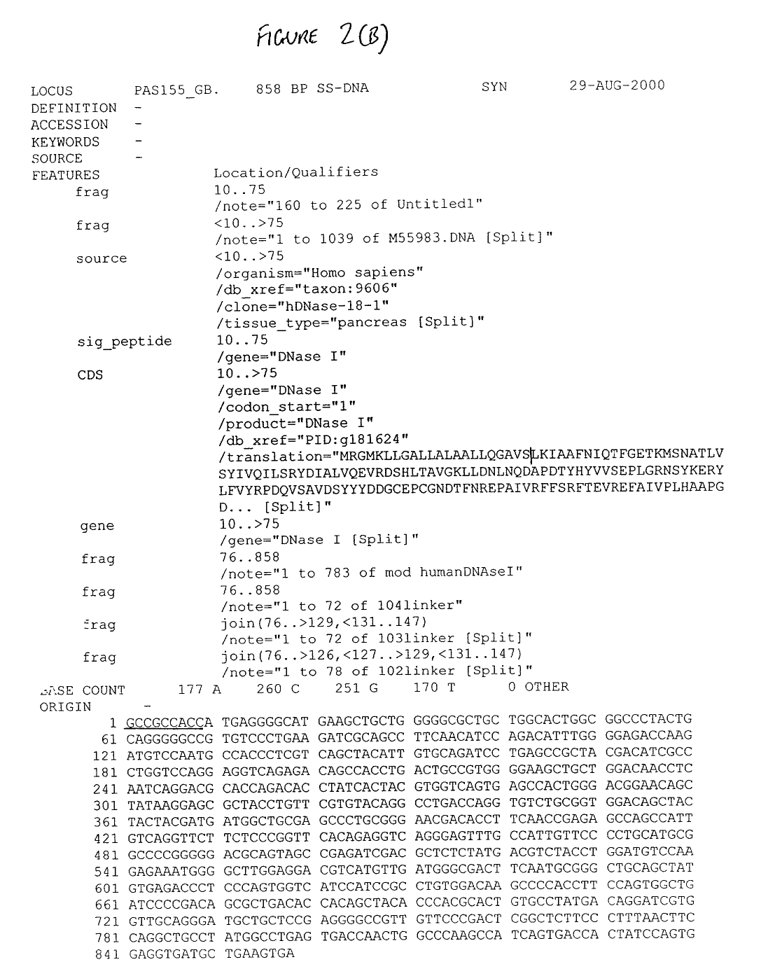

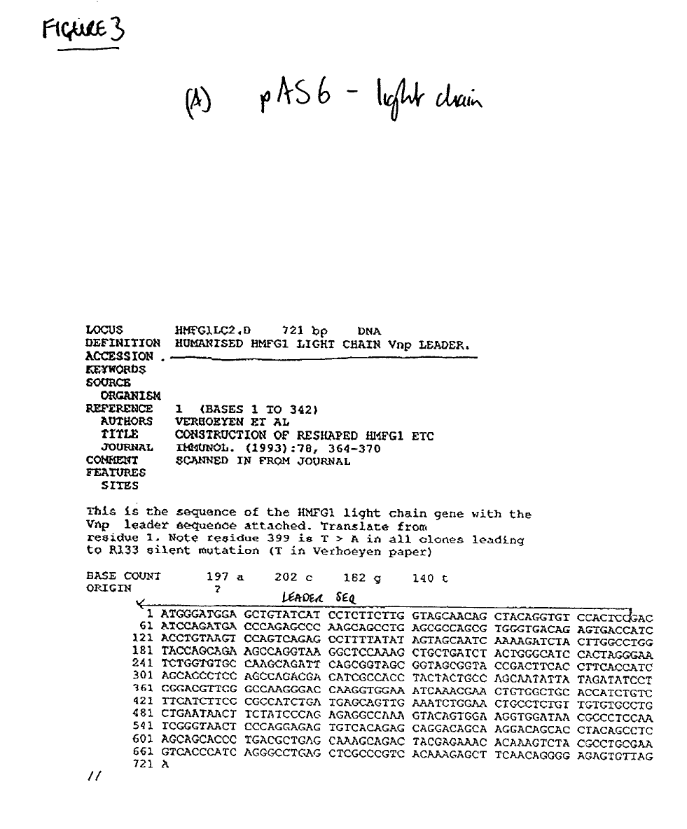

FIG. 1 shows the complete coding sequence of human DNAse I.

FIG. 2 shows (A) the mature DNAse peptide I sequence used in the exemplary Ab-DNase and Fab-DNase constructs, and (B) a truncated DNAse peptide I sequence encoded by a nucleotide sequence comprising a Kozak sequence (underlined).

FIG. 3 shows (A) the nucleotide sequence encoding the humanised HMFG1 light chain including leader peptide, (B) the nucleotide sequence of (A) further comprising a Kozak sequence (underlined), (C) the amino acid sequence of the humanised HMFG1 light chain including leader peptide (shaded) and (D) the nucleotide sequence encoding the humanised HMFG1 heavy chain including leader peptide,

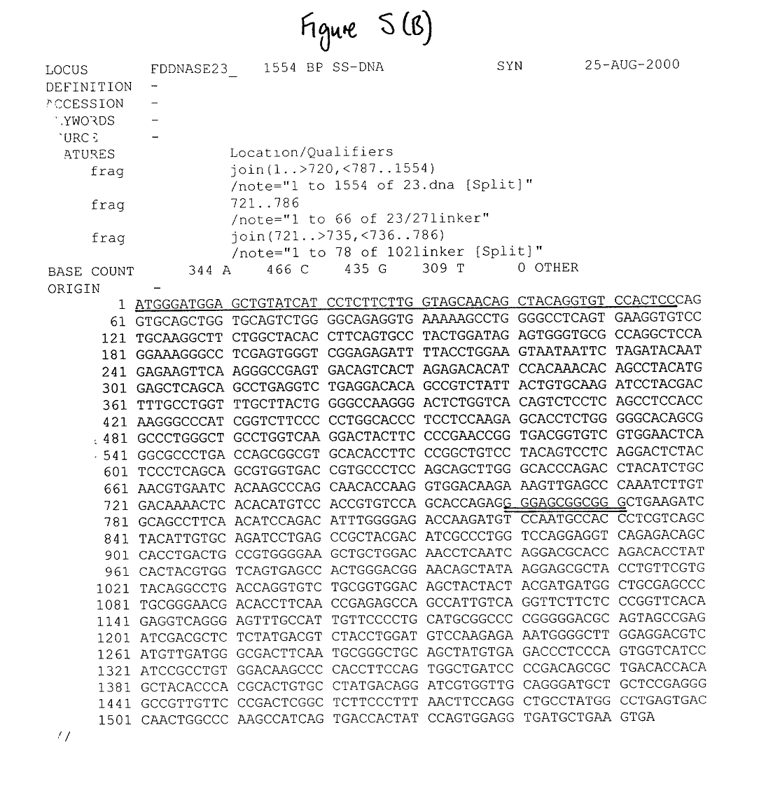

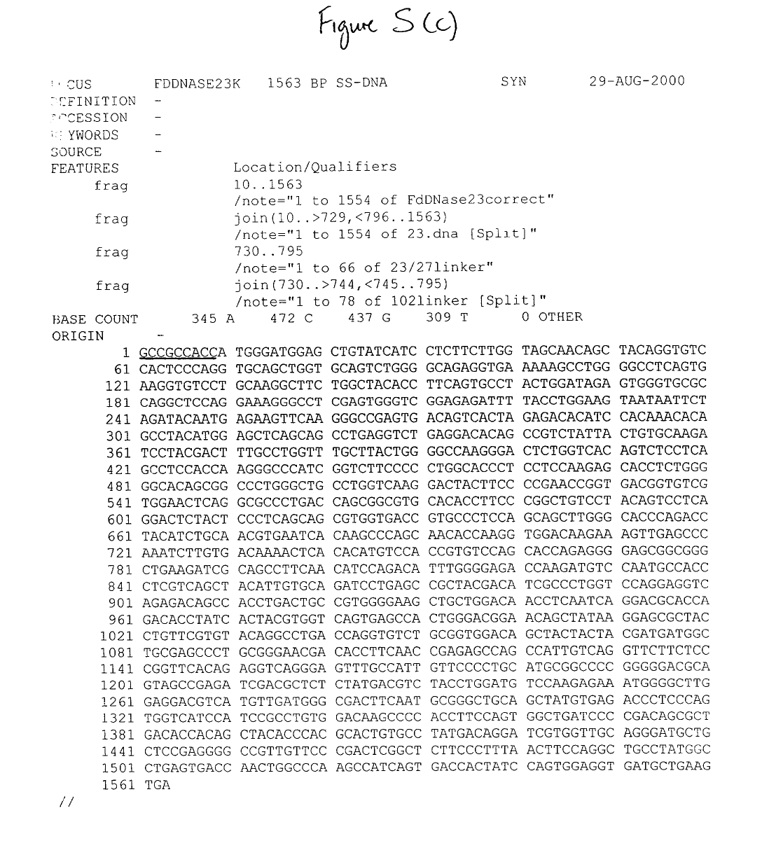

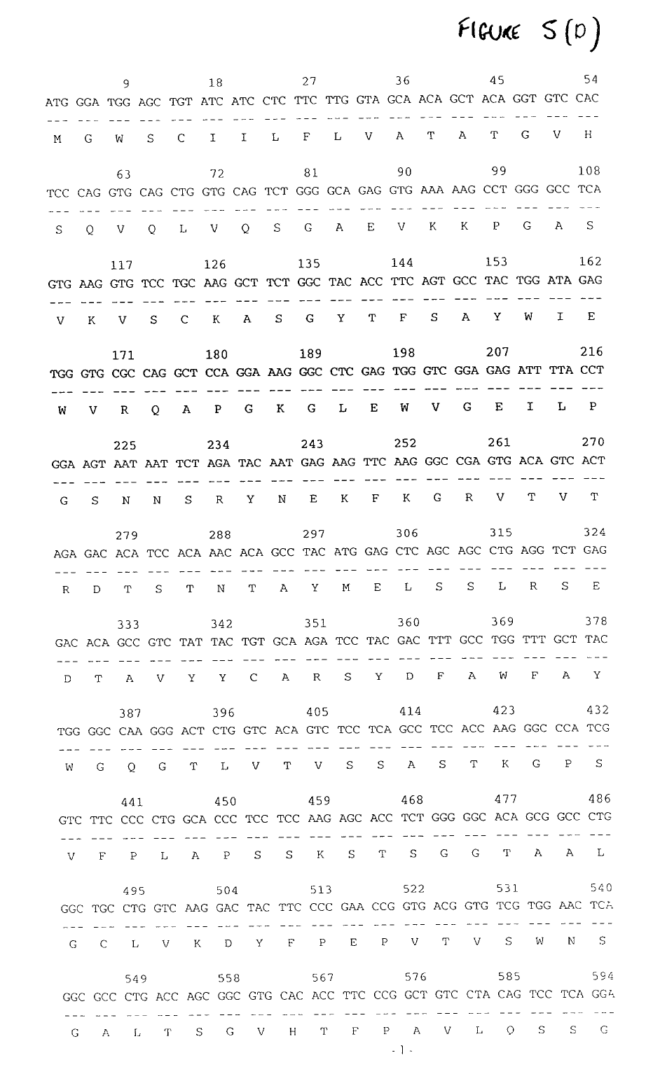

FIG. 4 shows the linker and hinge-linker oligonucleotides used in (A) the whole antibody-DNase and (B) the Fd-DNase exemplary constructs. Note, in FIG. 4(A) a deletion of one or more codons between the HMFG1 hinge and the linker is represented as ΔG.

FIG. 5 shows nucleotide sequences (A and B) encoding a humanised HMFG-1 Fd/DNase I fusion pAS23 comprising a leader sequence (underlined) and a linker sequence (double-underlined). FIG. 5(C) shows the nucleotide sequence of (B) further comprising a Kozak sequence (underlined). Figure (D) shows the amino acid sequence of a humanised HMFG-1 Fd/DNase I fusion.

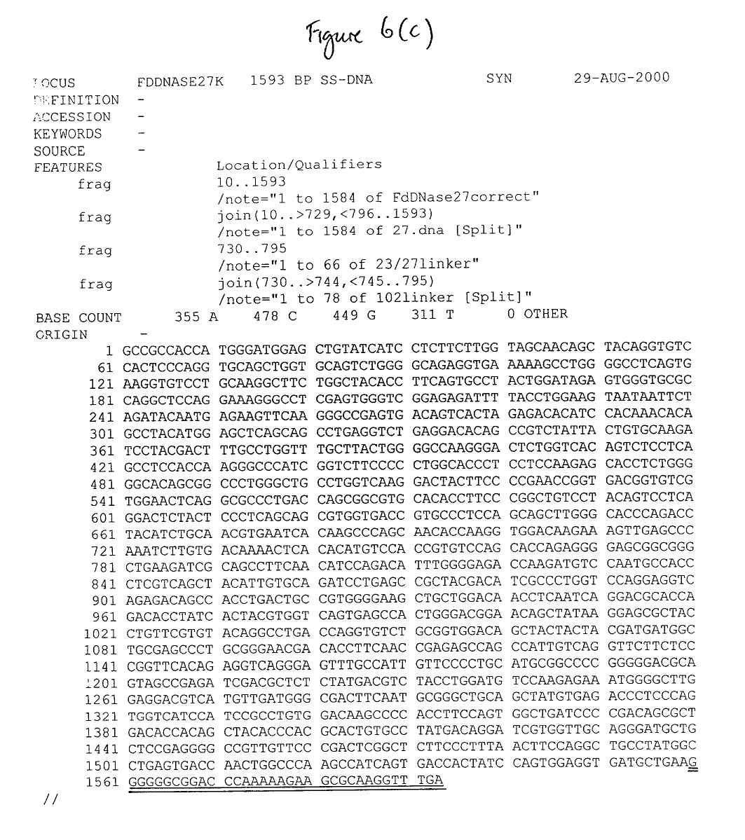

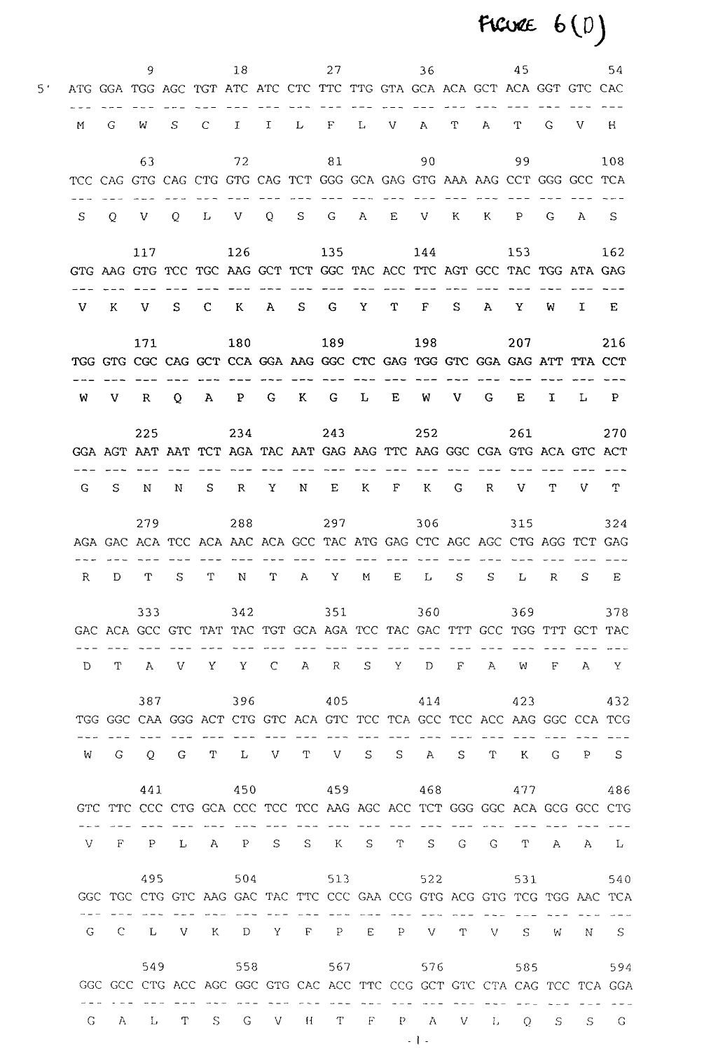

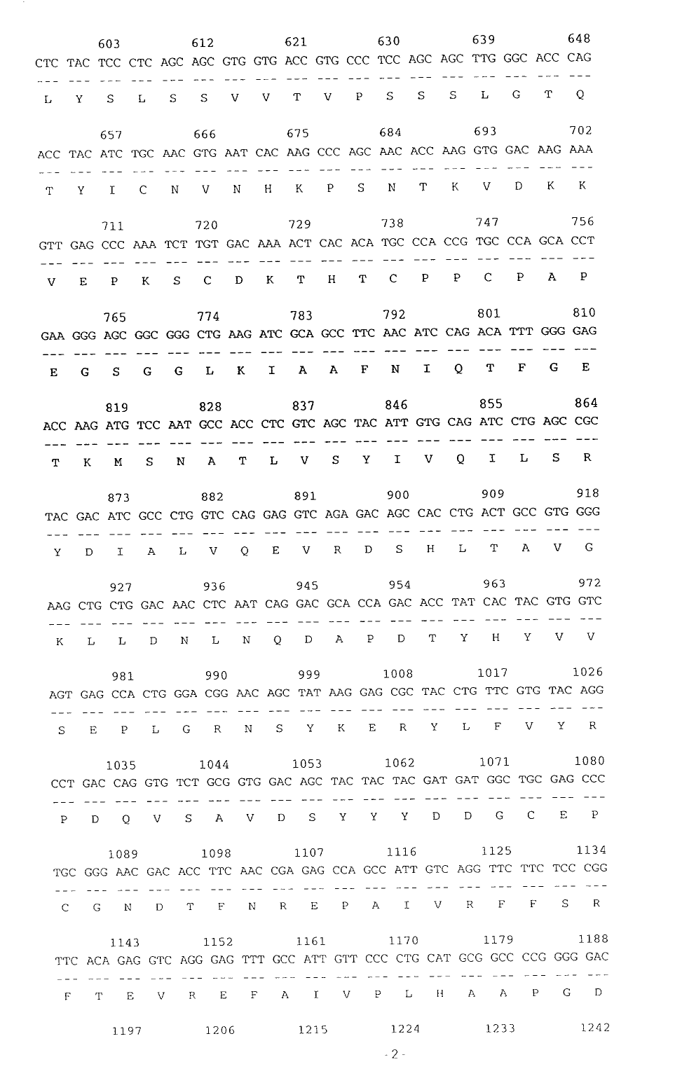

FIG. 6 shows (A), (B) and (C) shows the nucleotide sequences of FIG. 5 (A), (B) and (C), respectively, further comprising an SV40 NLS (double underlined) (pAS27). Figure (D) shows the amino acid sequence of a humanised HMFG-1 Fd/DNase I fusion comprising an SV40 NLS (double underlined).

FIG. 7 shows (A) the nucleotide sequence and (B) the translated amino acid sequence of an exemplary HMFG-1 heavy chain/DNase I fusion pAS34 (as used in ‘Ab-DNase’ in Example 2), comprising a leader sequence (underlined) and a linker sequence (double-underlined).

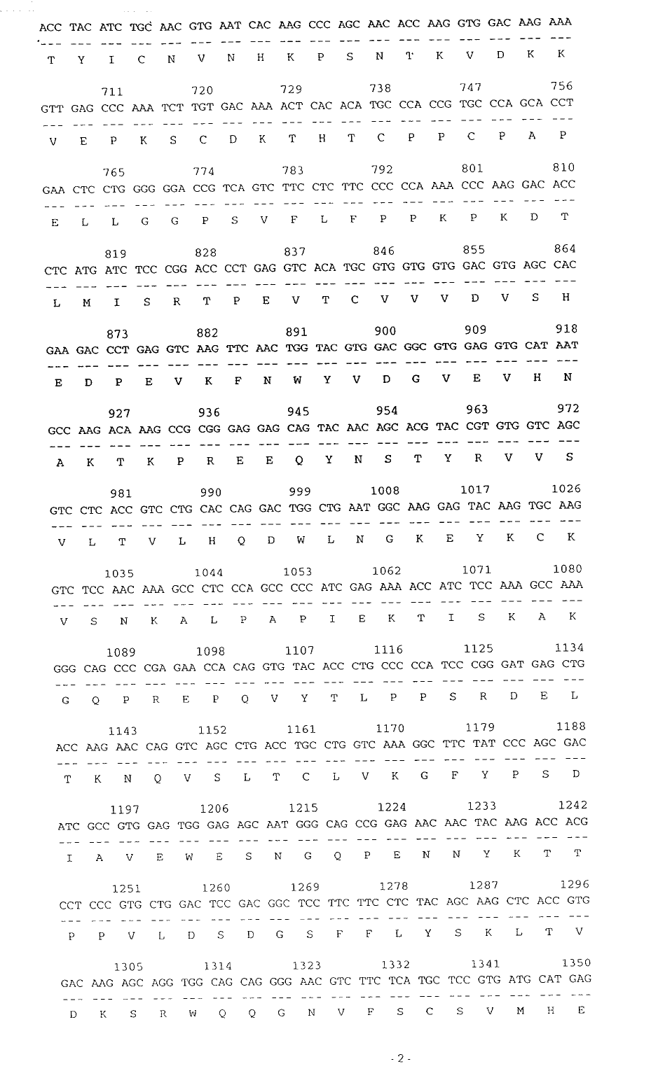

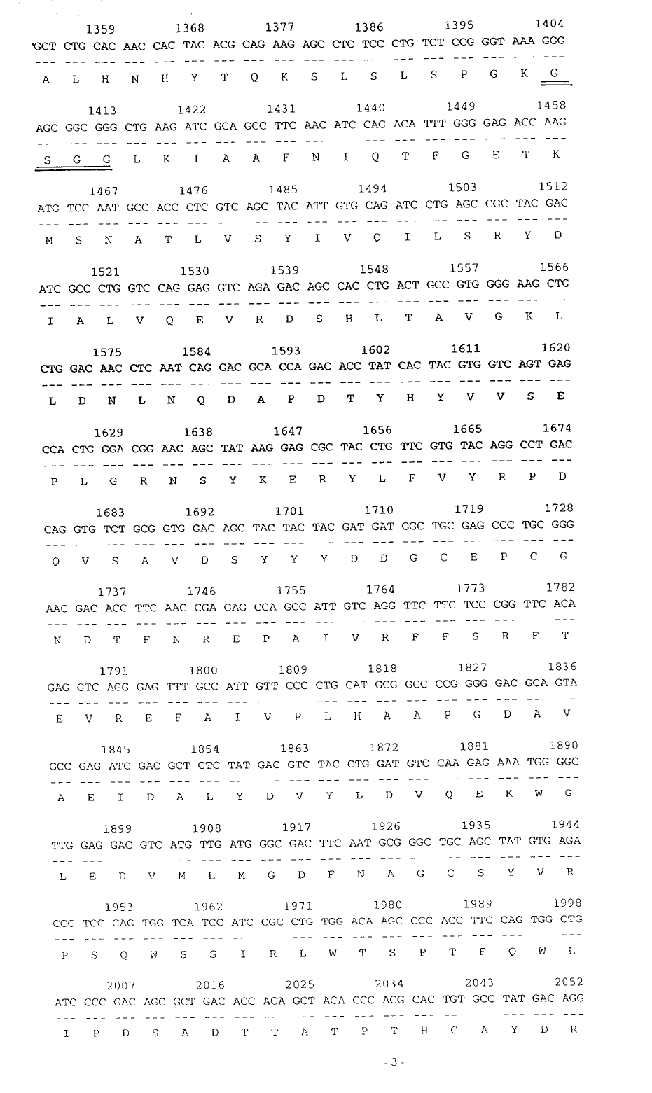

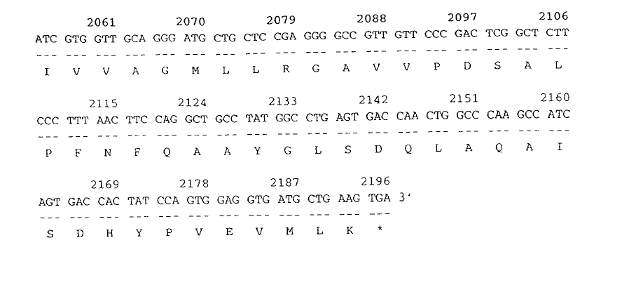

FIG. 8 shows (A) the nucleotide sequence and (B) the translated amino acid sequence of an exemplary HMFG-l heavy chain/DNase I fusion pAS35, comprising a leader sequence (underlined) and a linker sequence (double-underlined). The lower case ‘g’ represents a silent mutation caused by PCR amplification.

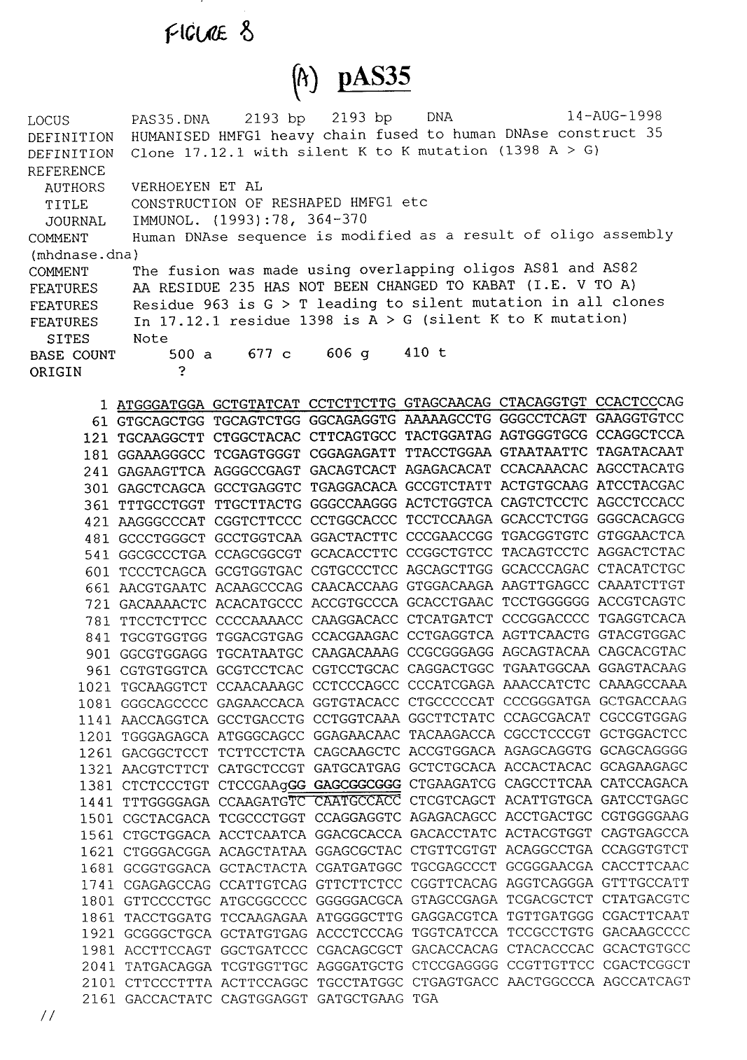

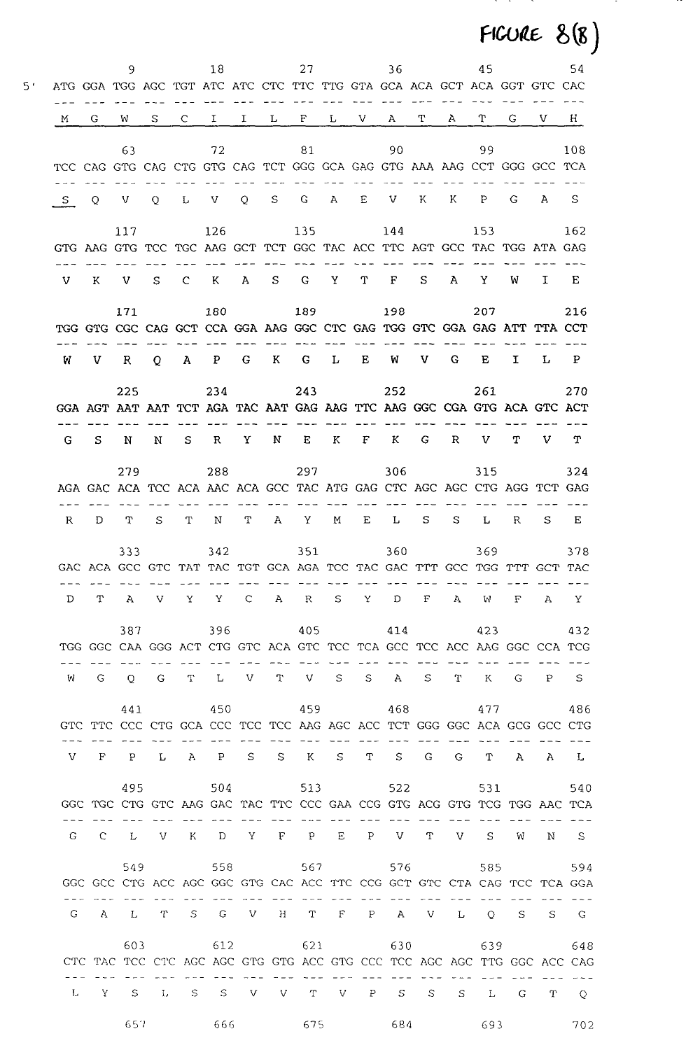

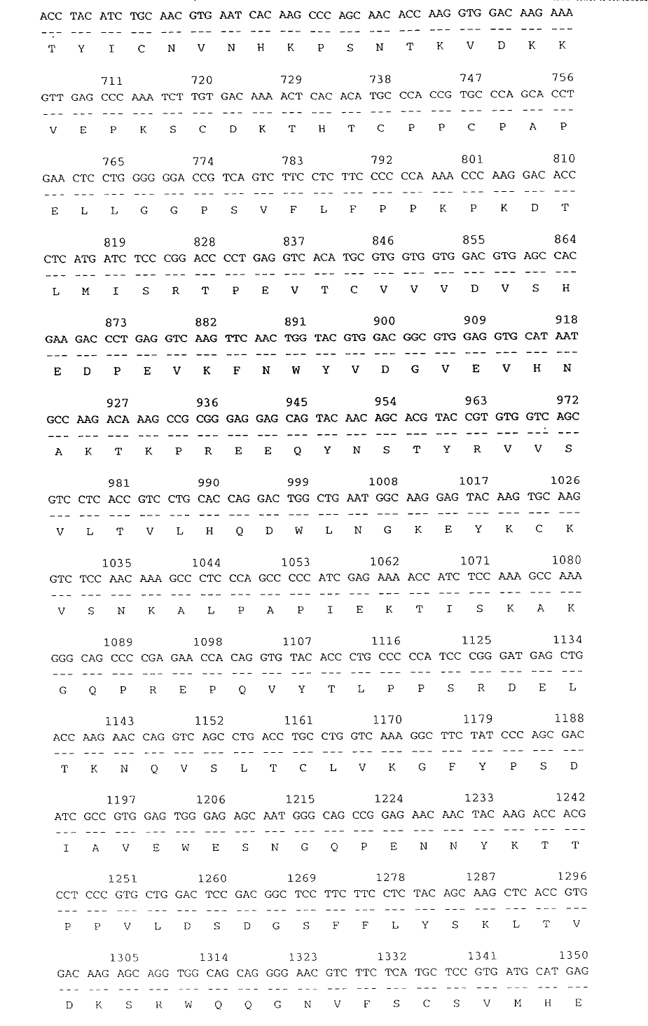

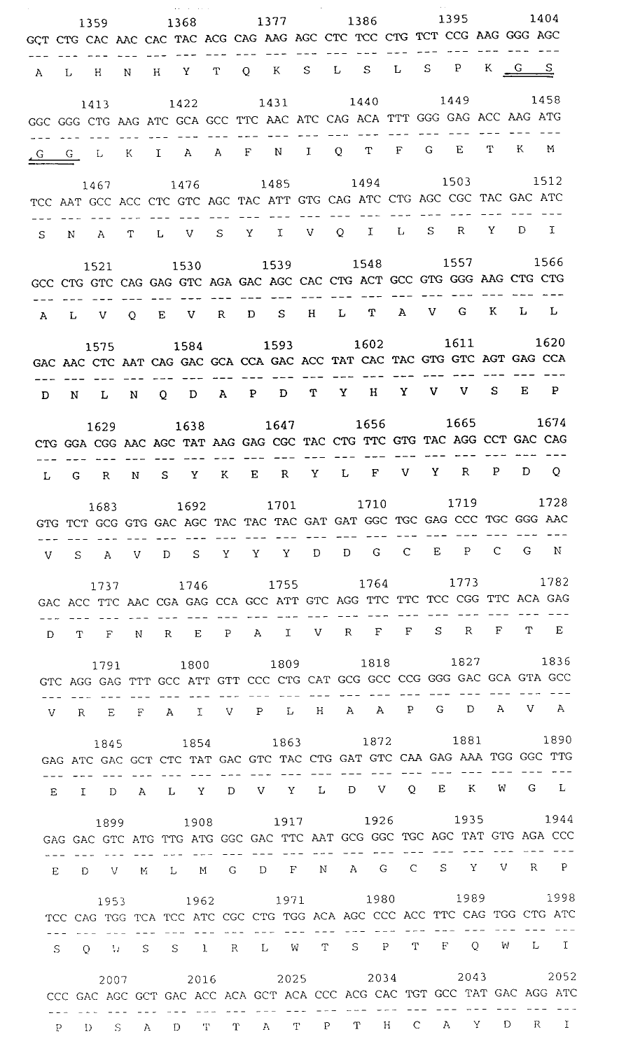

FIG. 9 shows (A) the nucleotide sequence and (B) the translated amino acid sequence of an exemplary HMFG-1 heavy chain/DNase I fusion pAS36, comprising a leader sequence (underlined) and a linker sequence (double-underlined). The lower case ‘c’ represents a silent mutation caused by PCR amplification.

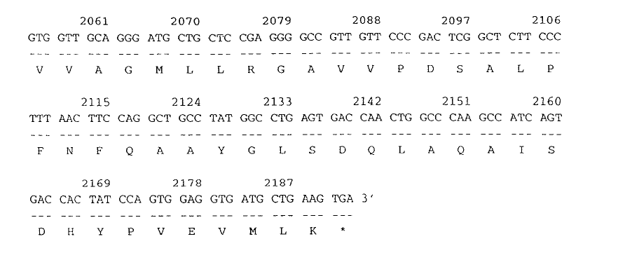

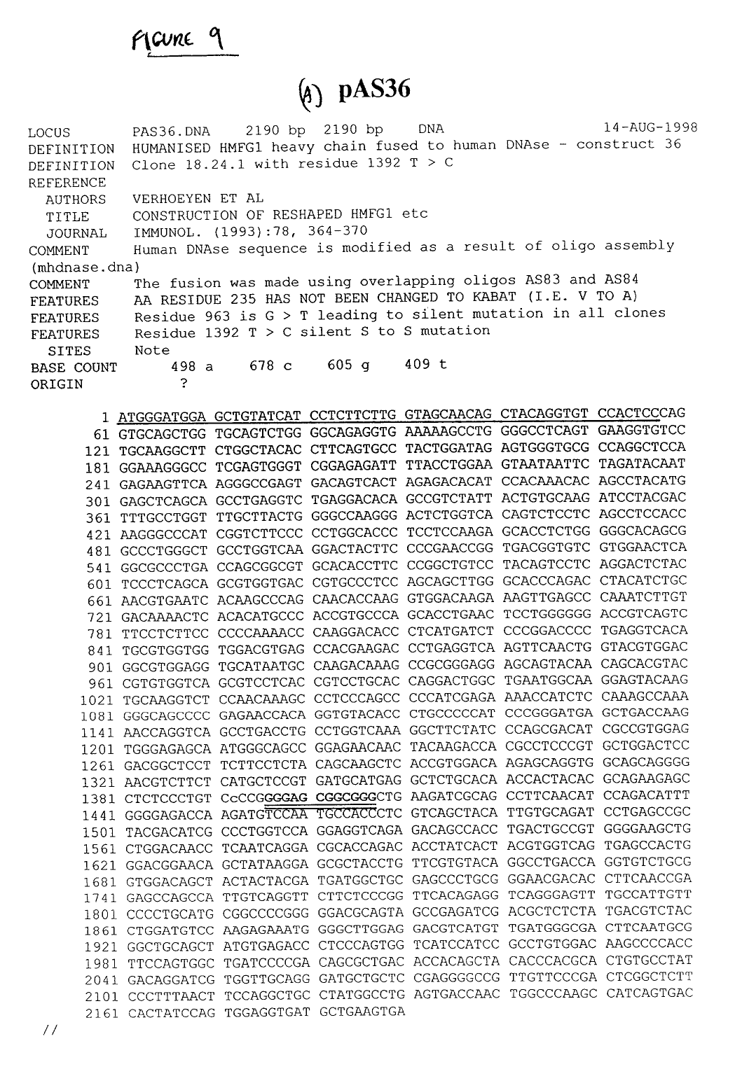

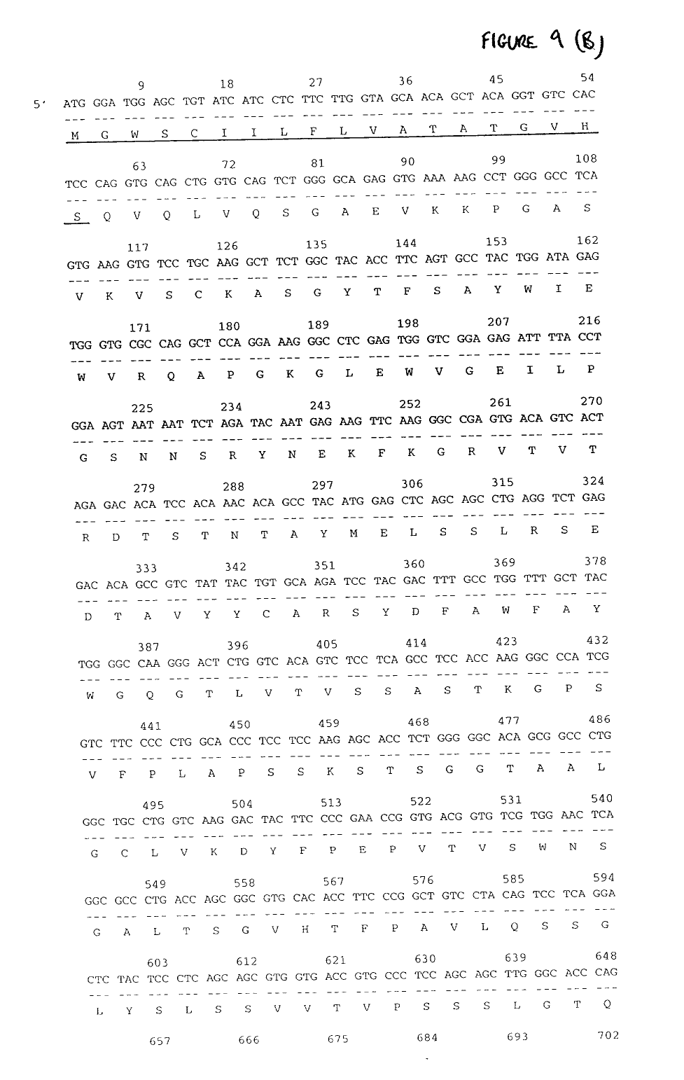

FIG. 10 shows (A) the nucleotide sequence and (B) the translated amino acid sequence of an exemplary HMFG-1 heavy chain/DNase I fusion pAS37, comprising a leader sequence (underlined), a linker sequence (double-underlined) and an NLS sequence (triple underlined).

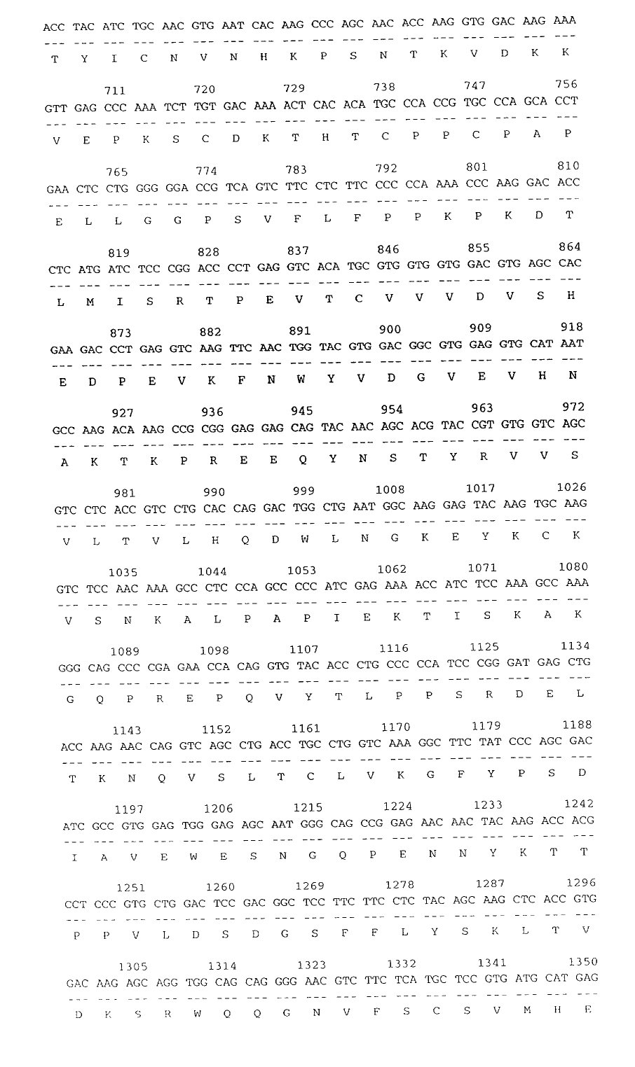

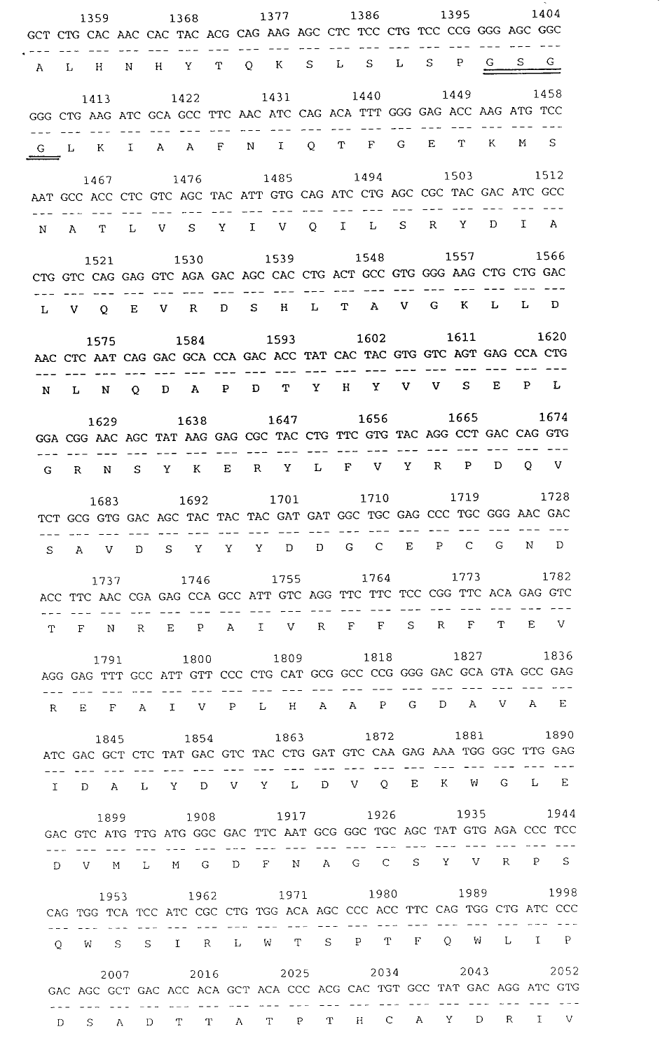

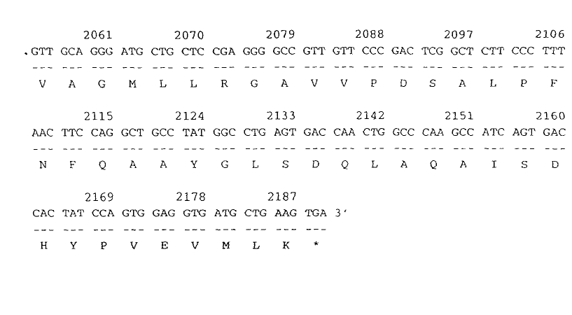

FIG. 11 shows (A) the nucleotide sequence and (B) the translated amino acid sequence of an exemplary HMFG-1 heavy chain/DNase I fusion pAS38, comprising a leader sequence (underlined), a linker sequence (double-underlined) and an NLS sequence (triple underlined). The lower case ‘g’ represents a silent mutation caused by PCR amplification.

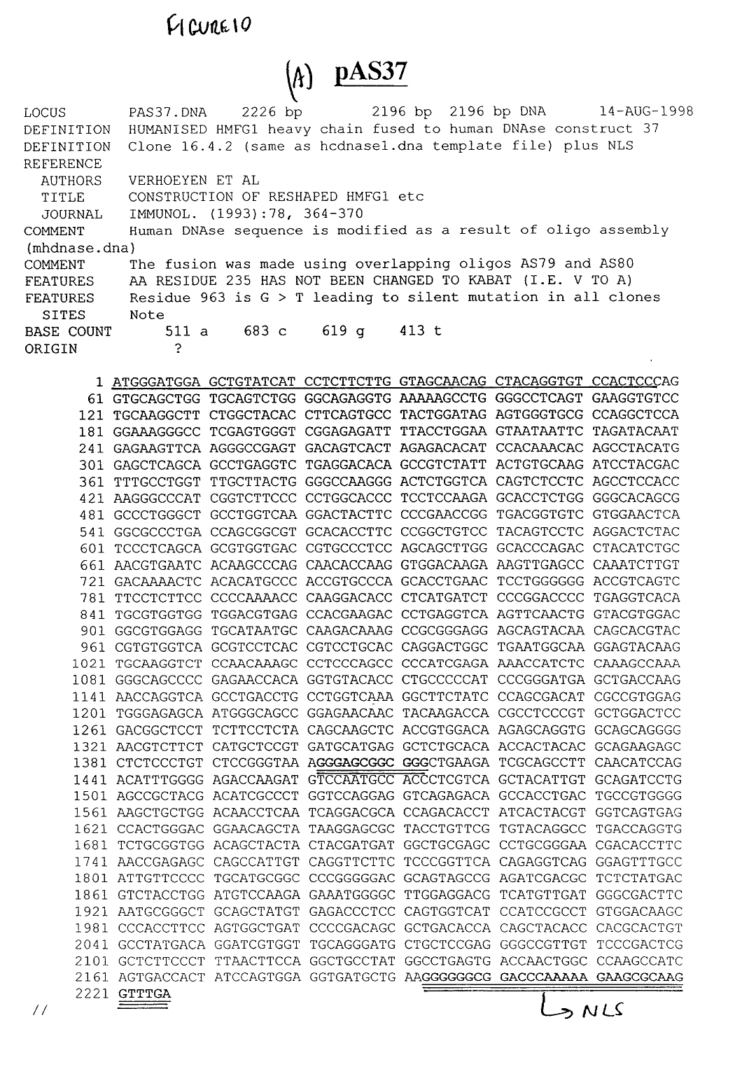

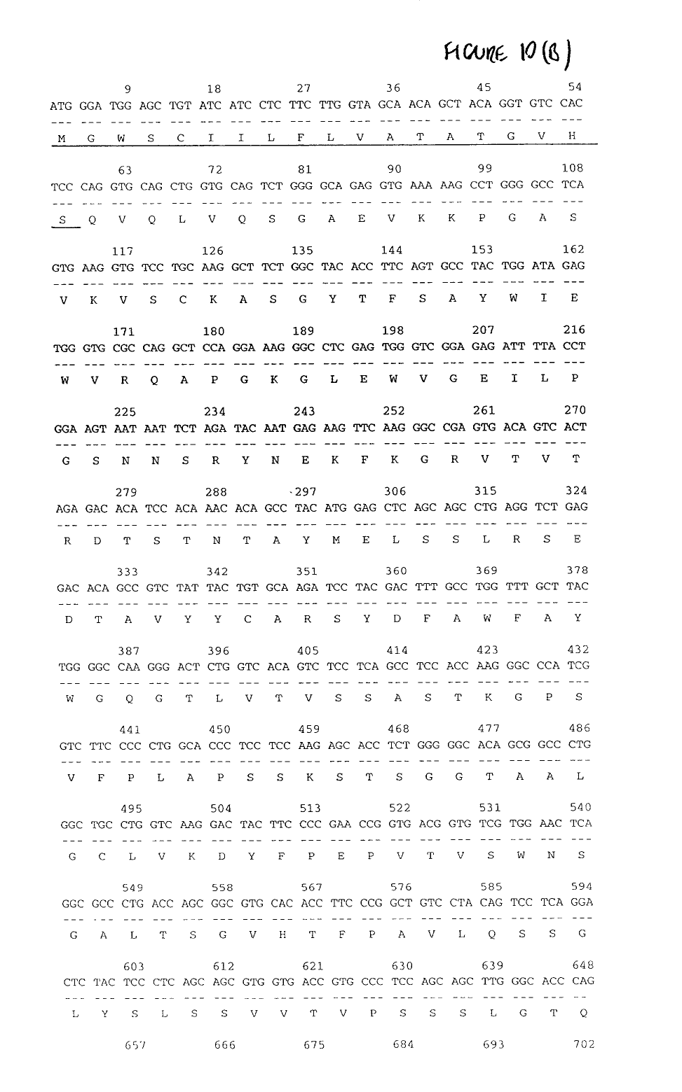

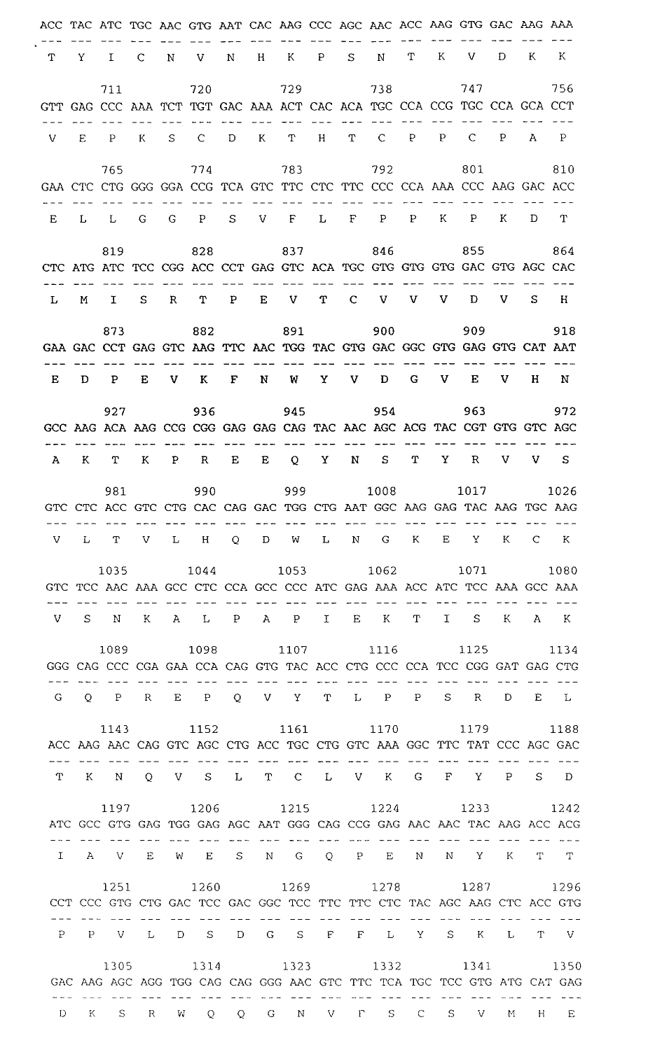

FIG. 12 shows (A) the nucleotide sequence and (B) the translated amino acid sequence of an exemplary HMFG-1 heavy chain/DNase I fusion pAS39, comprising a leader sequence (underlined), a linker sequence (double-underlined) and an NLS sequence (triple underlined). The lower case ‘c’ represents a silent mutation caused by PCR amplification.

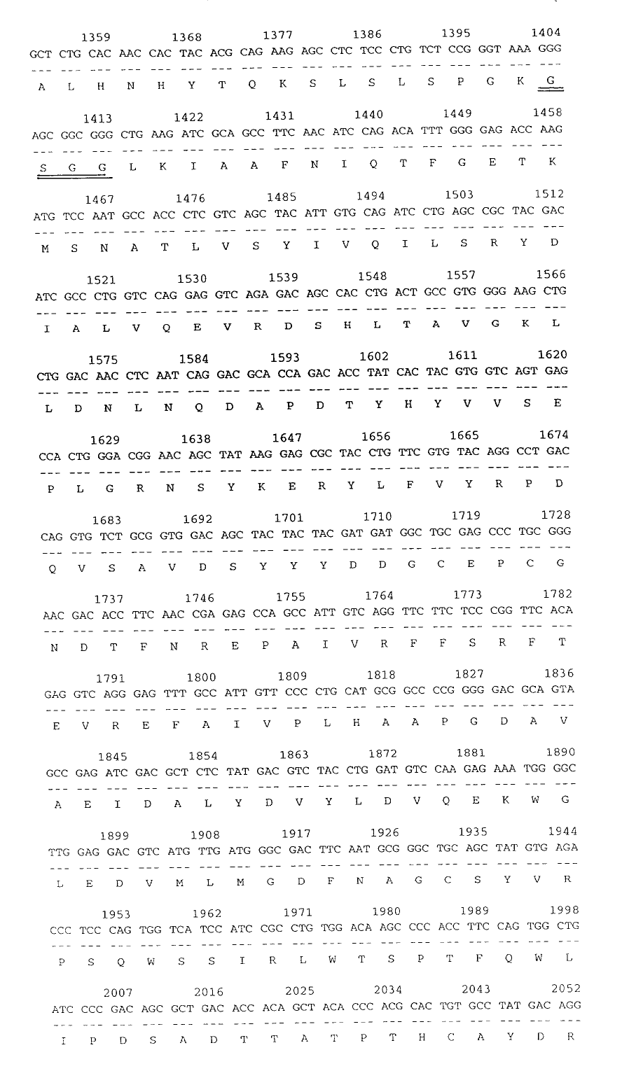

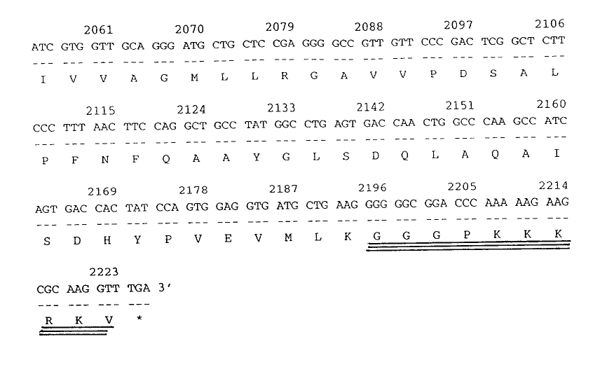

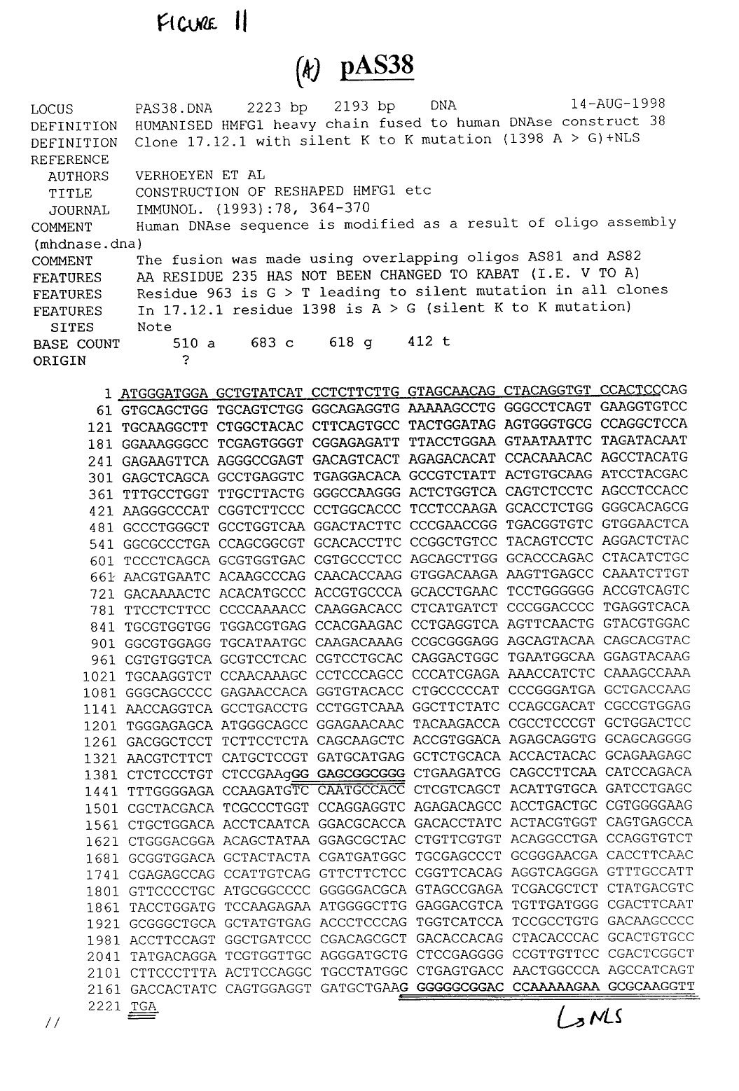

FIG. 13 shows nucleotide sequences (A and B) encoding a humanised HMFG-1 Fd/DNase I fusion pAS101 comprising a short leader sequence (underlined) and a linker sequence (double-underlined). FIG. 13(C) shows the nucleotide sequence of (B) further comprising a Kozak sequence (underlined). Figure (D) shows the amino acid sequence of a humanised HMFG-1 Fd/DNase I fusion.

FIG. 14 shows nucleotide sequences (A and B) encoding a humanised HMFG-1 Fd/DNase I fusion pAS102 comprising a leader sequence (underlined) and a hybrid hinge+linker sequence (double-underlined). FIG. 14(C) shows the nucleotide sequence of (B) further comprising a Kozak sequence (underlined) (construct designated pAS302 in Example 2). Figure (D) shows the amino acid sequence of a humanised HMFG-1 Fd/DNase I fusion.

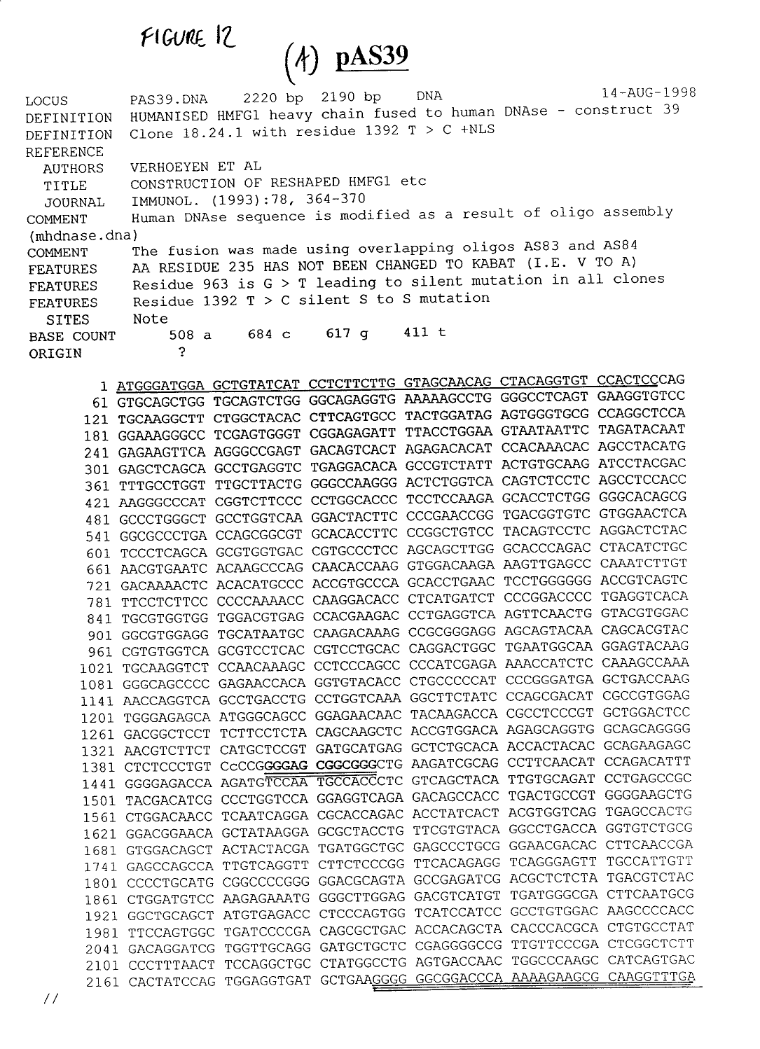

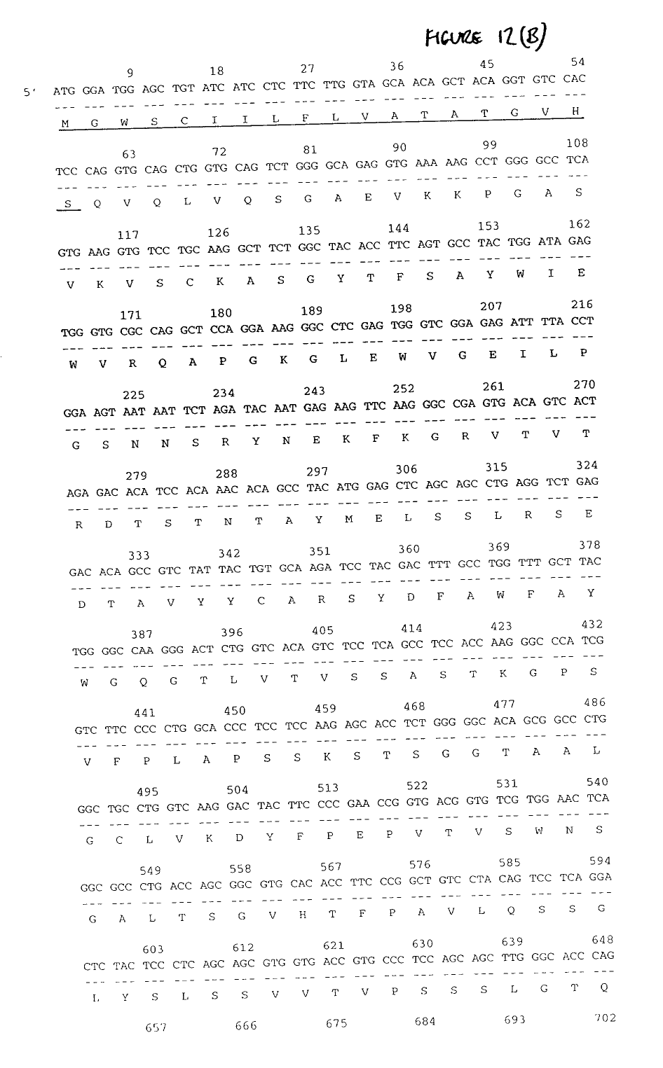

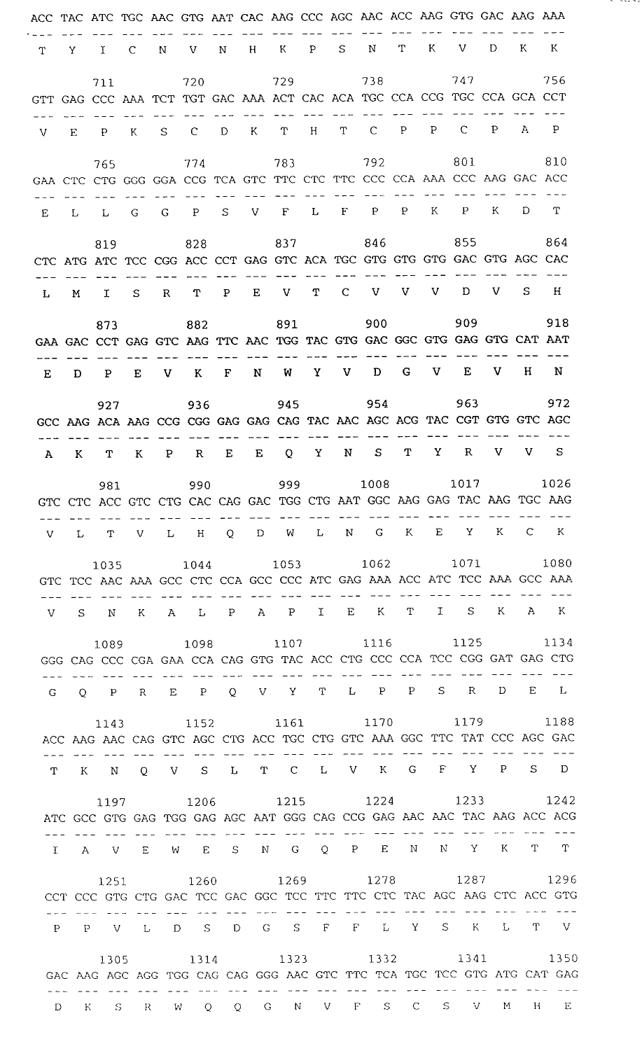

FIG. 15 shows nucleotide sequences (A and B) encoding a humanised HMFG-1 Fd/DNase I fusion pAS103 comprising a leader sequence (underlined) and a hybrid hinge+short linker sequence (double-underlined). FIG. 15(C) shows the nucleotide sequence of (B) further comprising a Kozak sequence (underlined). Figure (D) shows the amino acid sequence of a humanised HMFG-1 Fd/DNase I fusion.

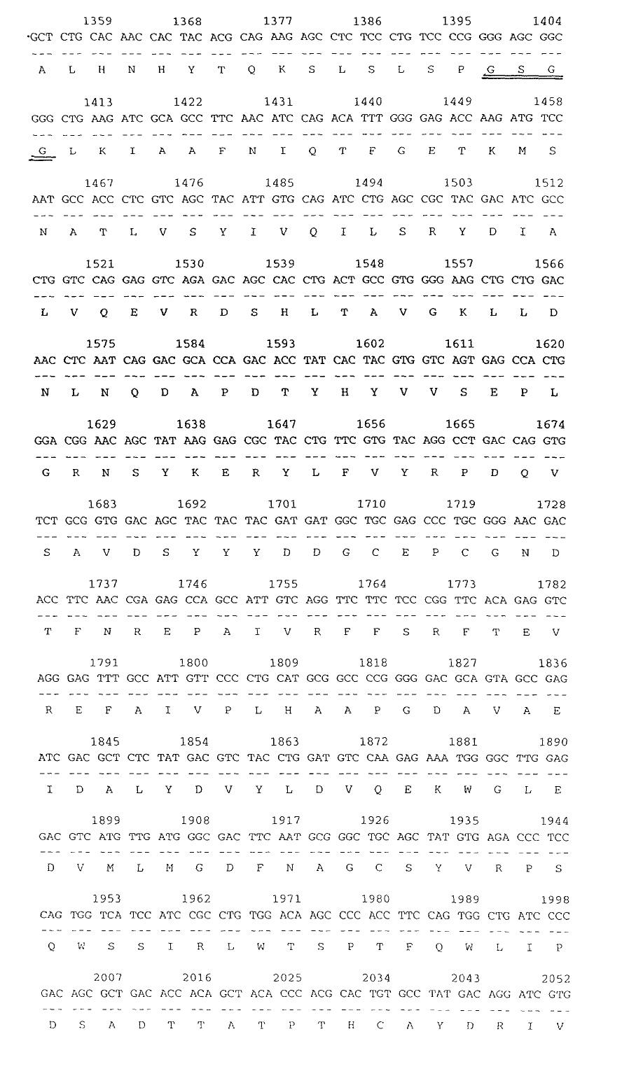

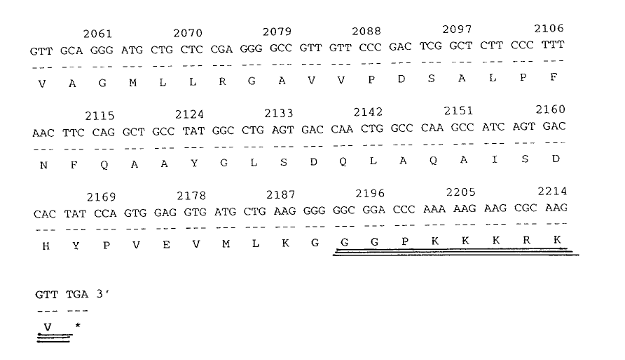

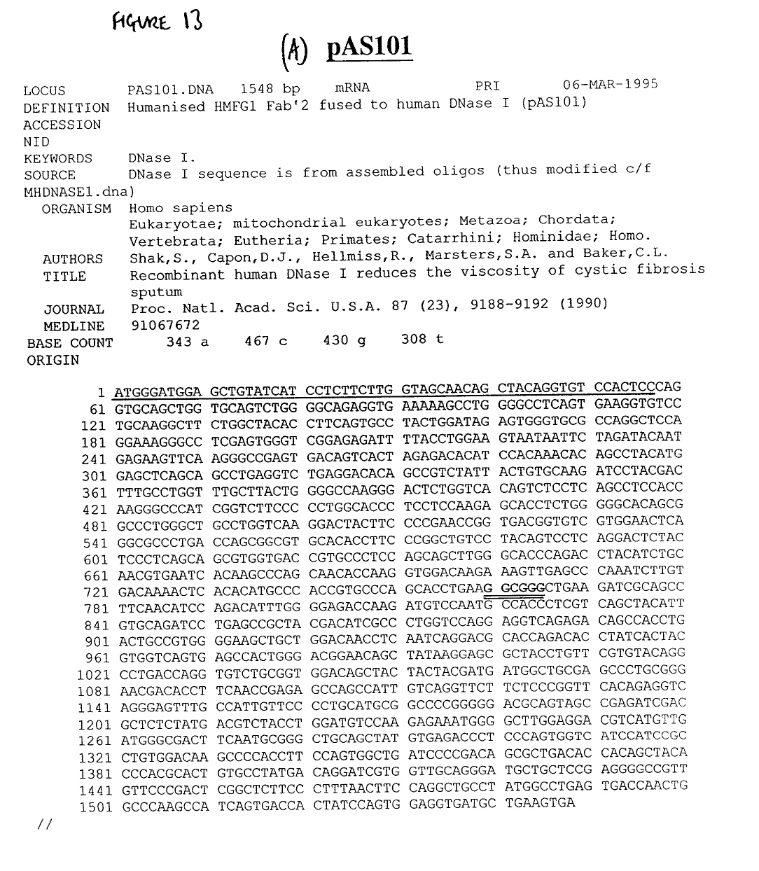

FIG. 16 shows nucleotide sequences (A and B) encoding a humanised HMFG-1 Fd/DNase I fusion pAS104 comprising a leader sequence (underlined) and a hybrid hinge+mutated short linker sequence (double-underlined). Figure (C) shows the amino acid sequence of a humanised HMFG-1 Fd/DNase I fusion. Mutations (compared to pAS103) at positions 775 and 924 are shaded.

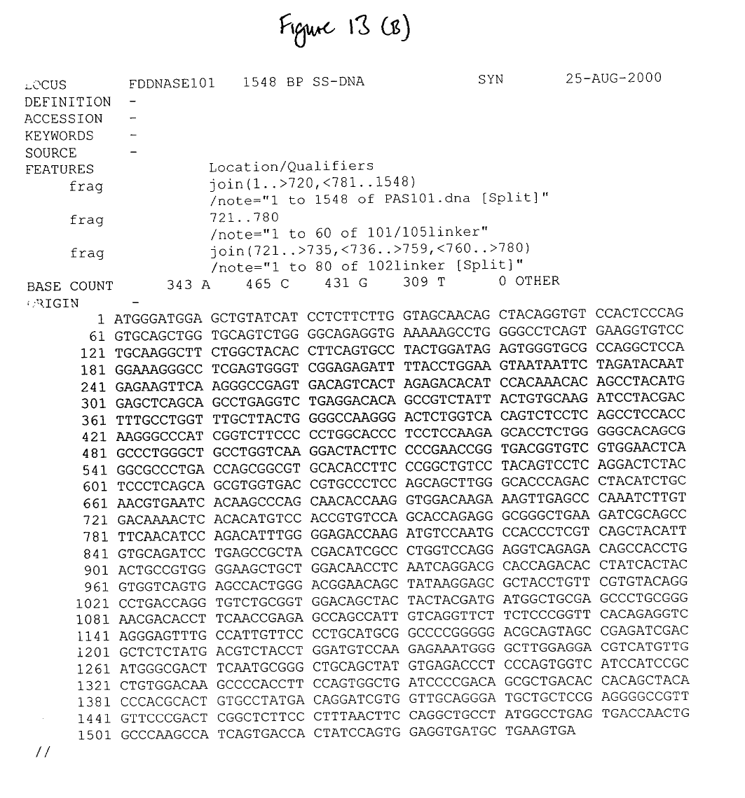

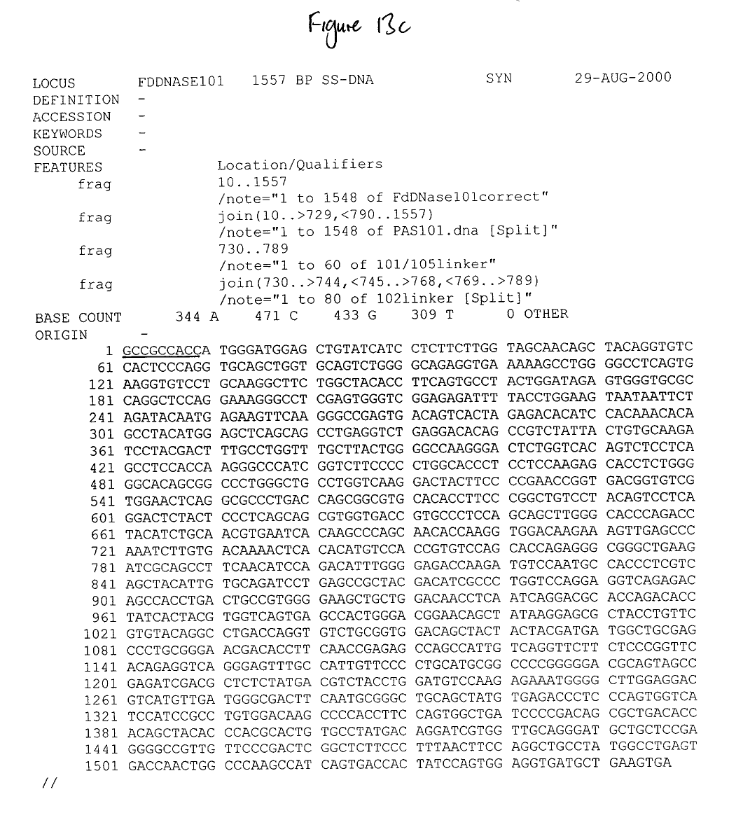

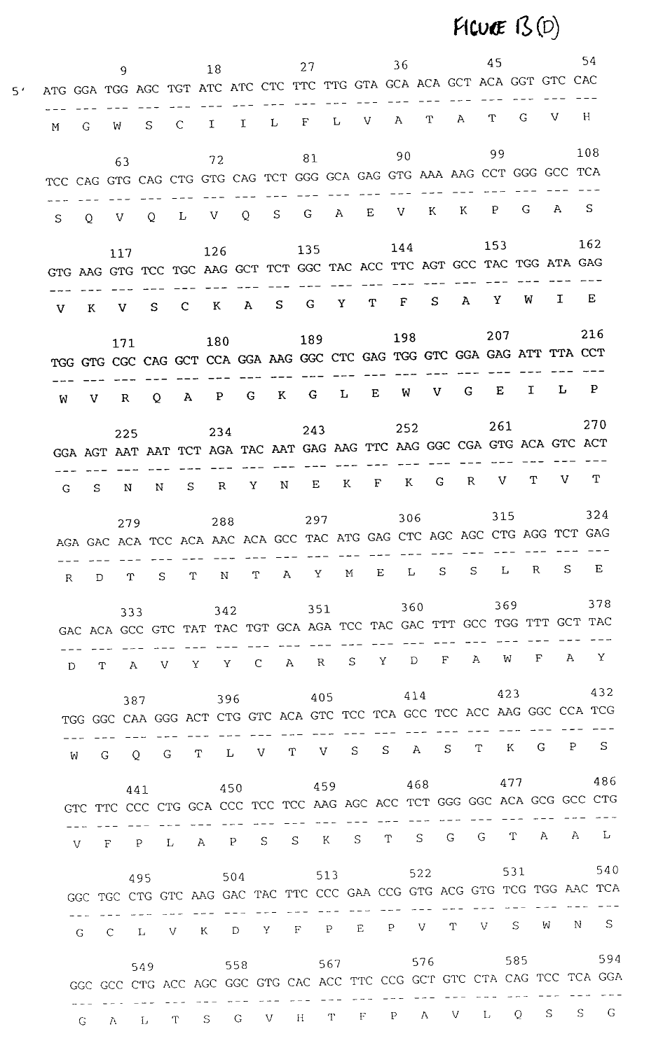

FIG. 17 shows nucleotide sequences (A and B) encoding a humanised HMFG-1 Fd/DNase I fusion pAS105 comprising a leader sequence (underlined), a short linker sequence (double-underlined) and an NLS sequence (triple underlined). FIG. 17(C) shows the nucleotide sequence of (B) further comprising a Kozak sequence (underlined). Figure (D) shows the amino acid sequence of a humanised HMFG-1 Fd/DNase I fusion.

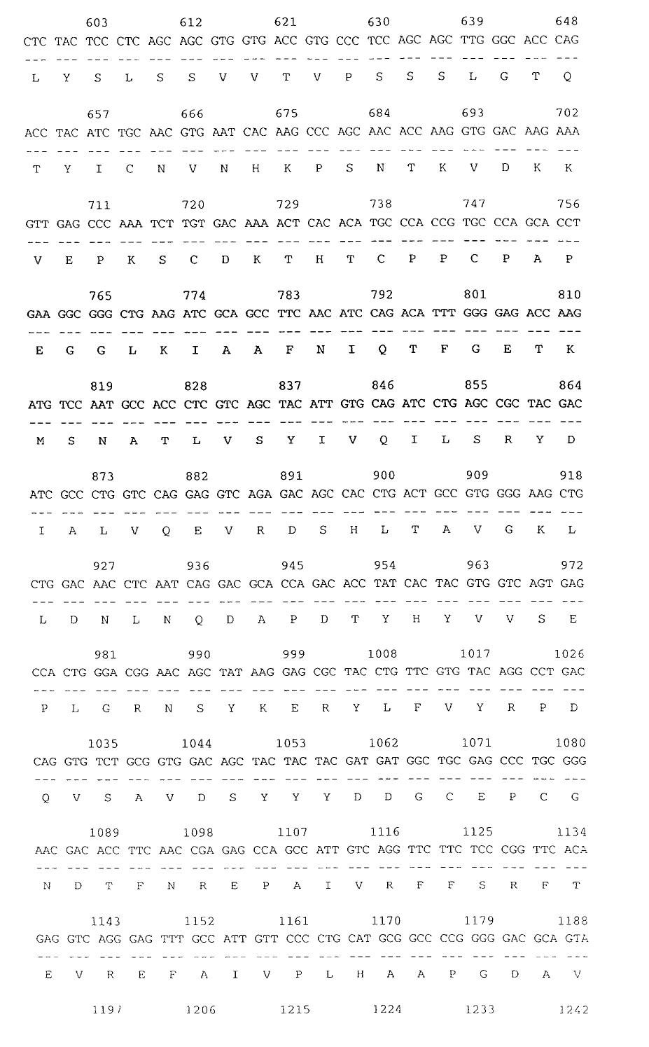

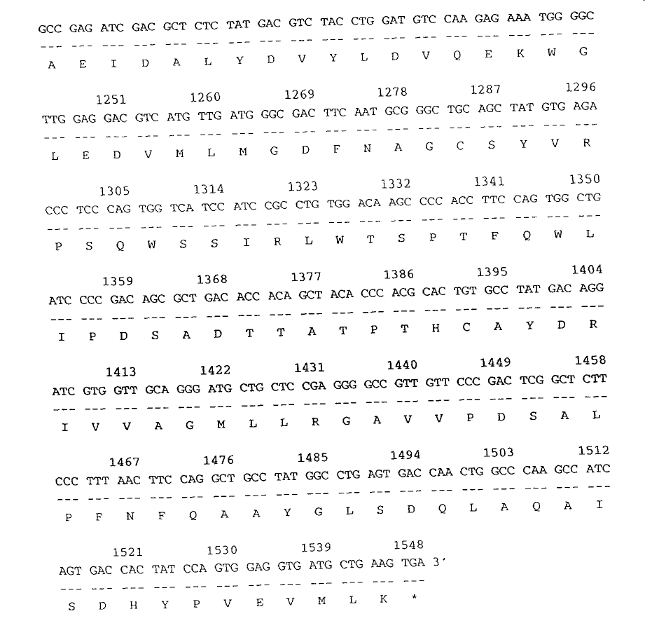

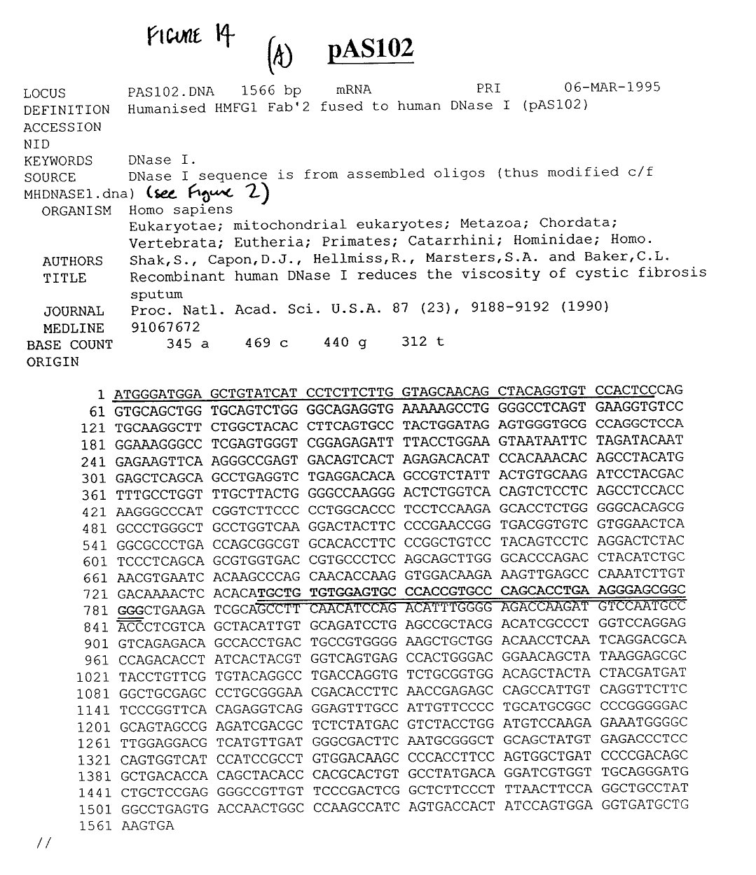

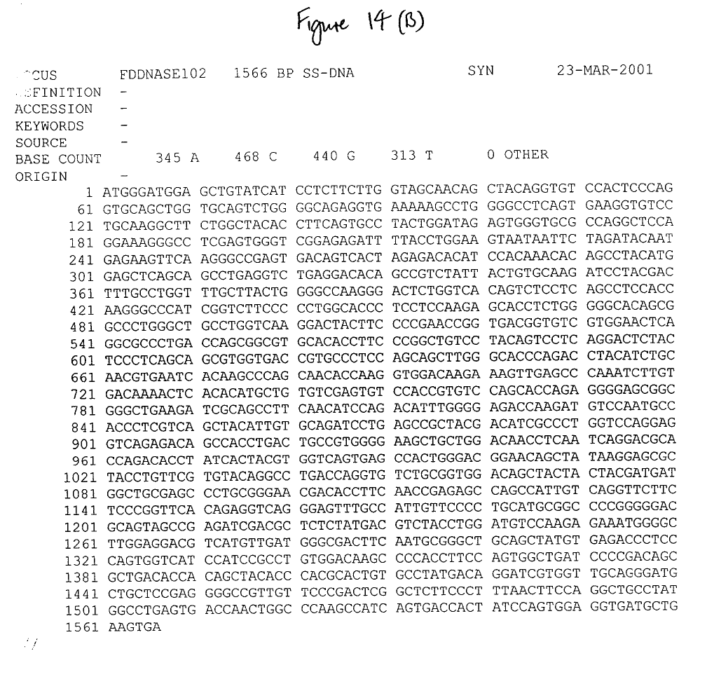

FIG. 18 shows nucleotide sequences (A and B) encoding a humanised HMFG-1 Fd/DNase I fusion pAS106 comprising a leader sequence (underlined), a hybrid hinge+linker sequence (double-underlined) and an NLS sequence (triple underlined). FIG. 18(C) shows the nucleotide sequence of (B) further comprising a Kozak sequence (underlined). Figure (D) shows the amino acid sequence of a humanised HMFG-1 Fd/DNase I fusion.

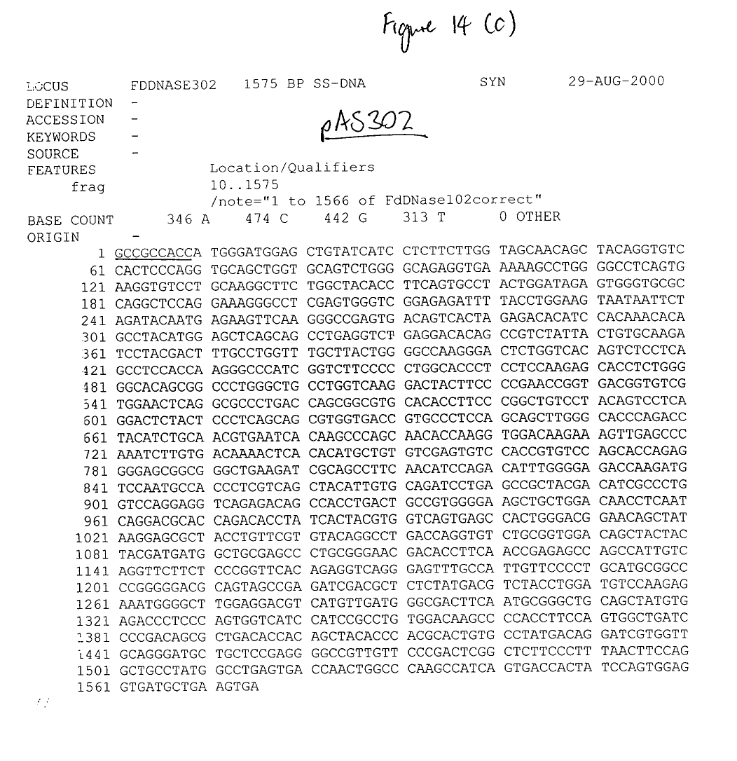

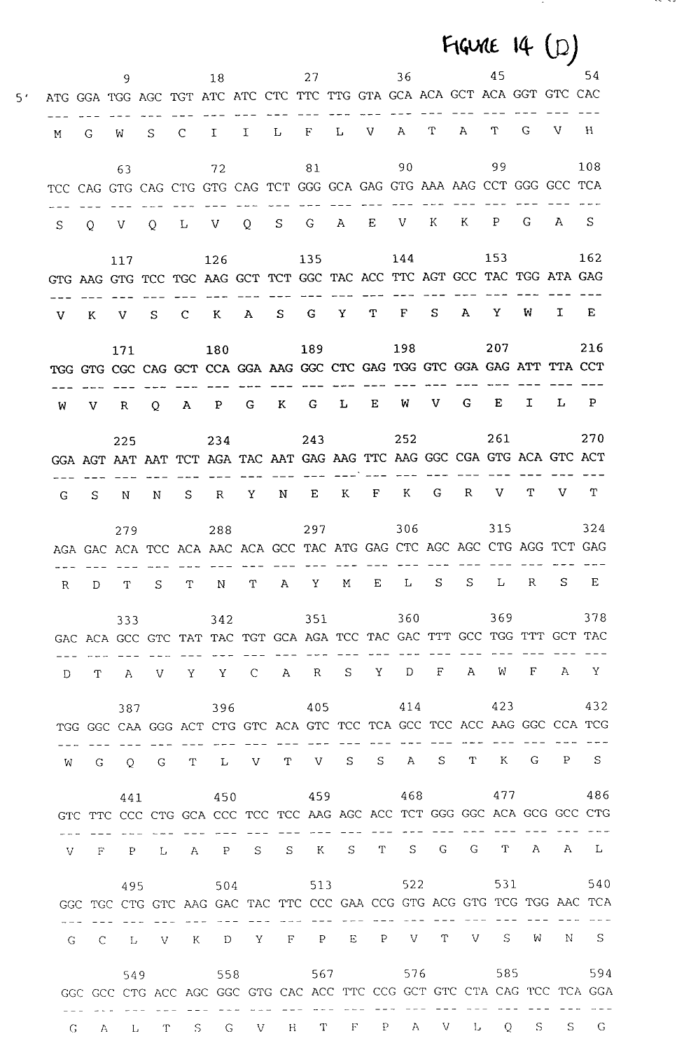

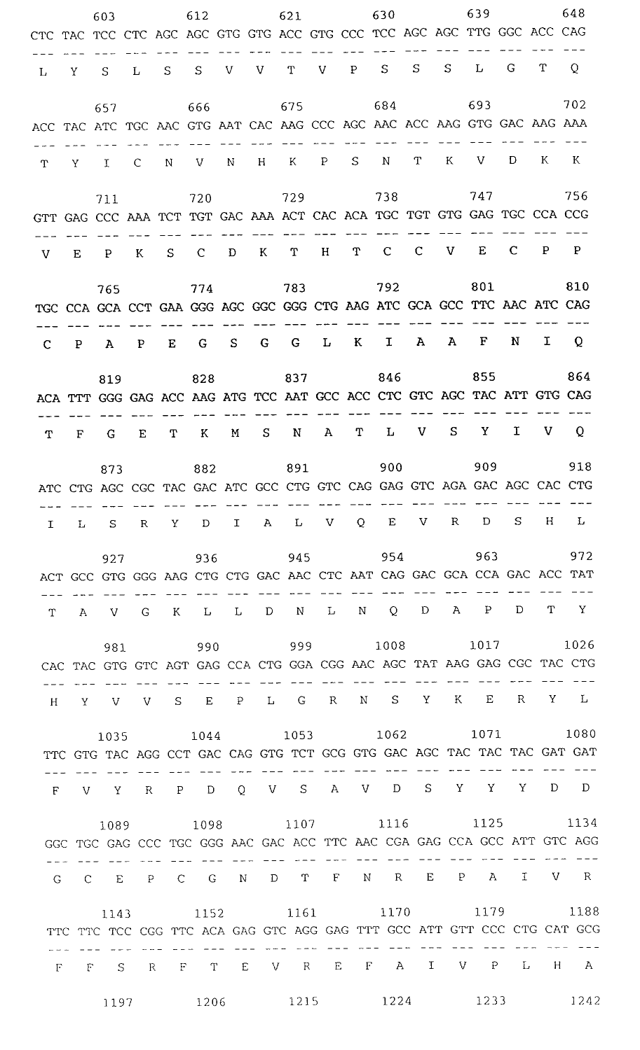

FIG. 19 shows nucleotide sequences (A and B) encoding a humanised HMFG-1 Fd/DNase I fusion pAS107 comprising a leader sequence (underlined), a hybrid hinge+short linker sequence (double-underlined) and an NLS sequence (triple underlined). FIG. 19(C) shows the nucleotide sequence of (B) further comprising a Kozak sequence (underlined). Figure (D) shows the amino acid sequence of a humanised HMFG-1 Fd/DNase I fusion.

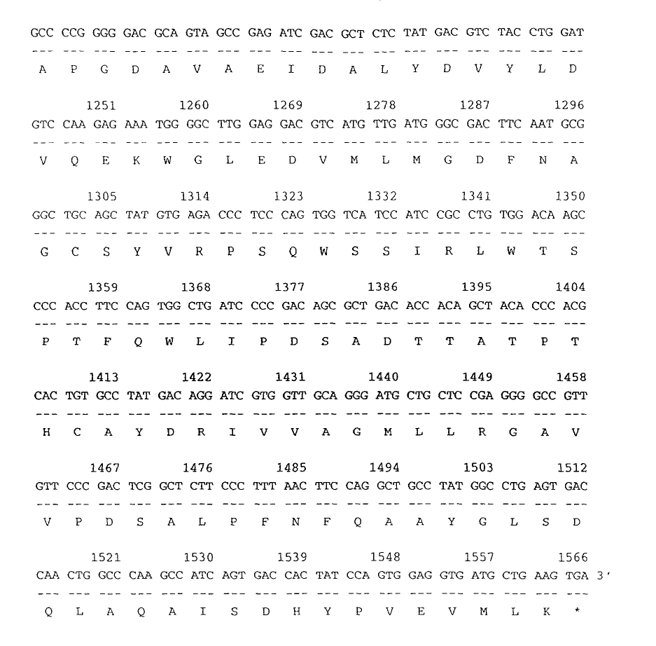

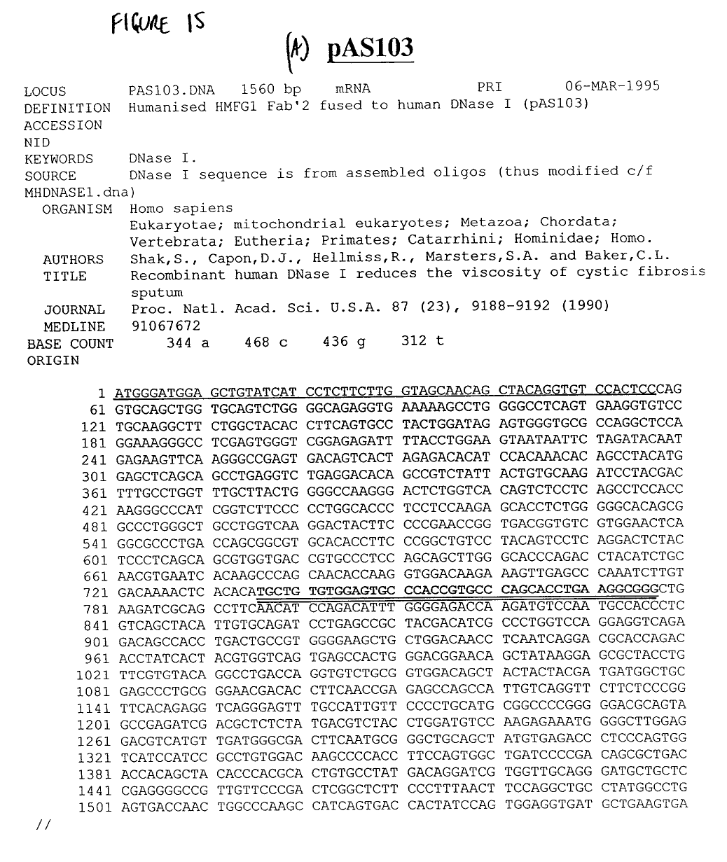

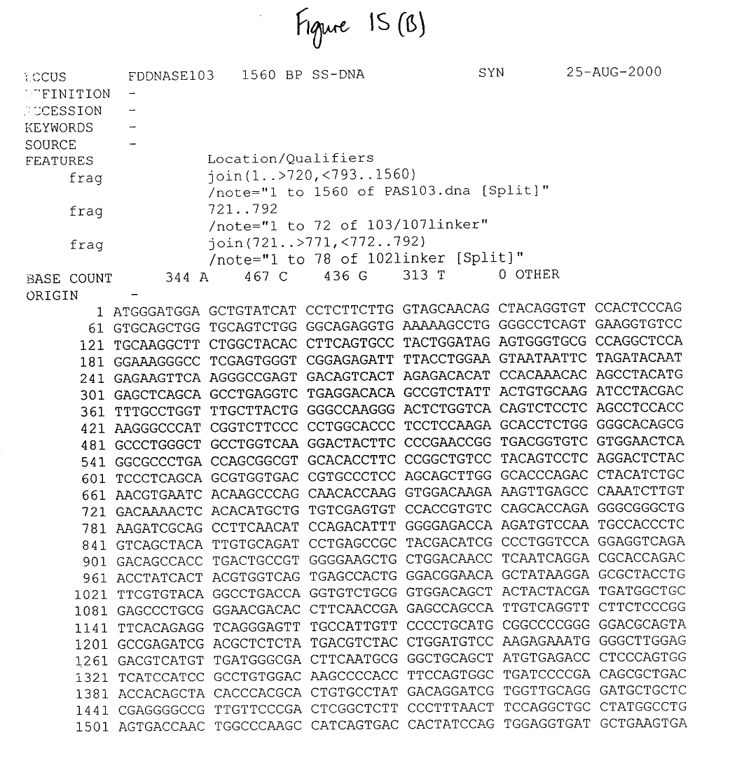

FIG. 20 shows a schematic diagram of the pEE6 expression vector used in the exemplary constructs.

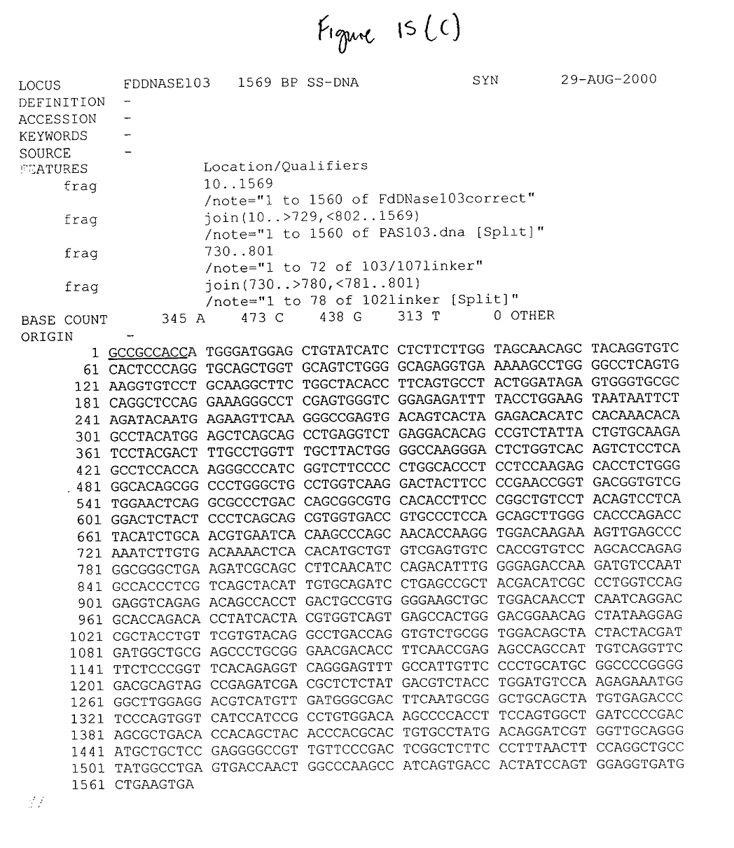

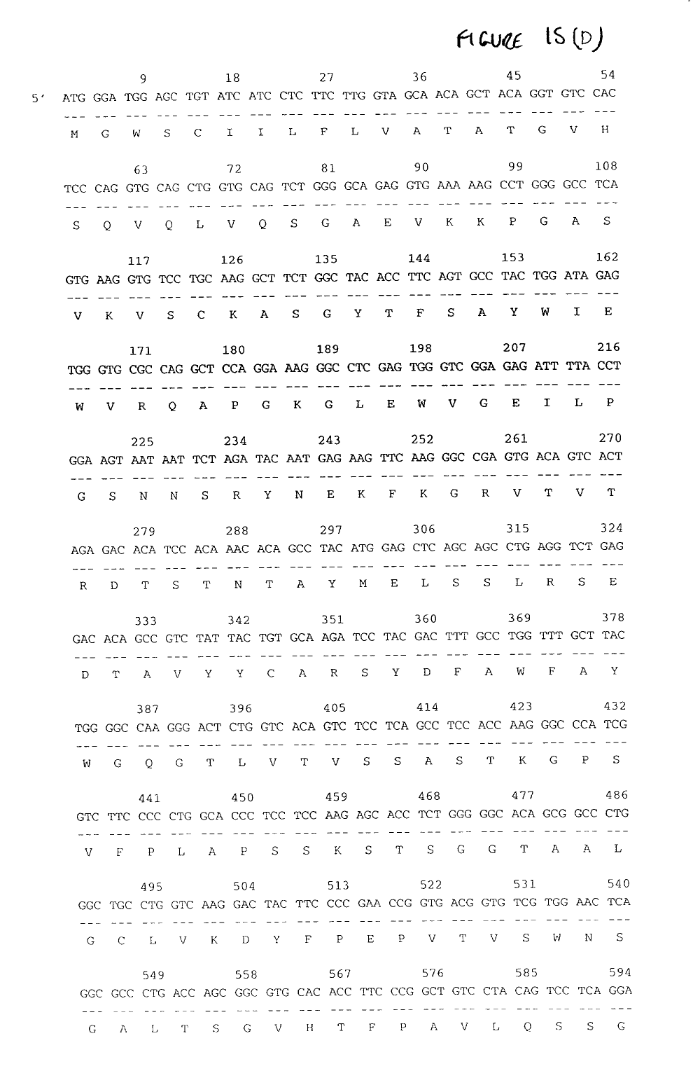

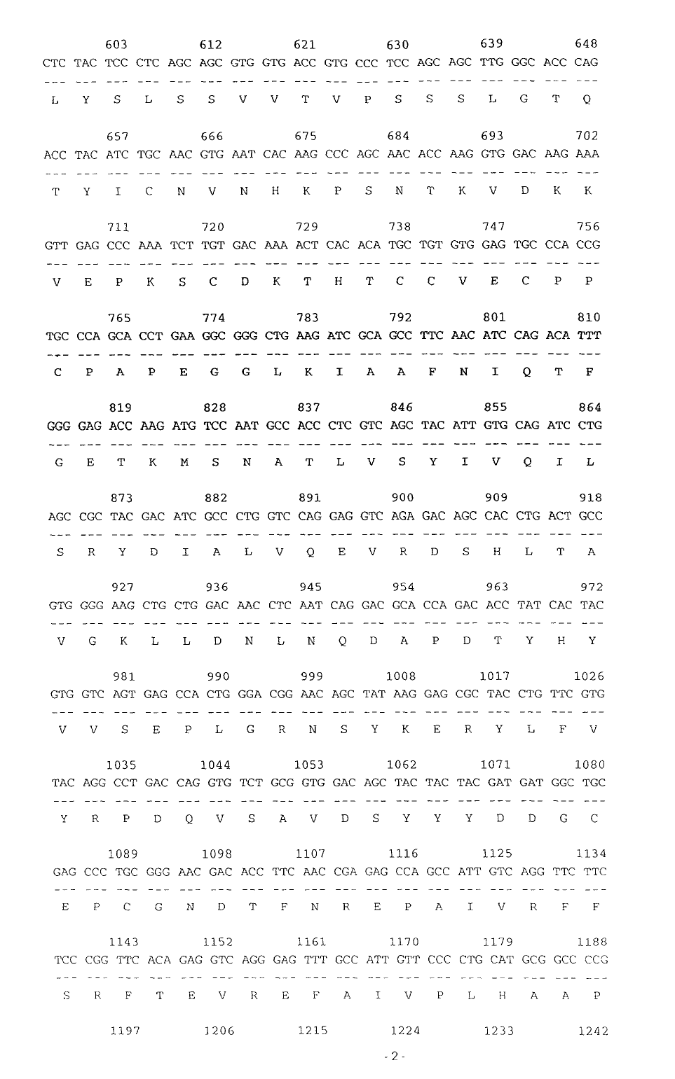

FIG. 21 shows autoradiographs from inmuno-precipitation experiments with metabolically labelled transient transfectants:

GEL A

Lane 1 shows the precipitation of supernatant from mock-transfected cells.

Lane 2 is from cells transfected with hHMFG-1 (construct 6) giving expected molecular weights of about 51.2 and 26.4 kDa for the heavy and light chains, respectively.

Lane 3 shows construct 34 antibody construct which has human DNase I fused to the C-terminus of the heavy chain gene. As expected, the size of the heavy chain gene has increased to about 80.7 kDa.

Samples from whole antibody DNase I constructs 35, 36 and 39 were run on the gel (Lanes 4 to 6) but were not sufficiently well expressed to be visible, in this experiment.

In subsequent experiments using this method, construct 39 was detectable but weak, and constructs 35 and 36 were detectable but very weak. Constructs 37 and 38 have not been tested in this assay system.

Lanes 8 to 10 are fusion of humanised HMFG1 F(ab′)₂ with human DNase I (constructs 41, 23 and 102, respectively). F(ab′)₂ alone was included in this set of experiments (lane 7, construct 41) but did not express, this was included in later experiments (see gels C and D). In addition to the light chain (about 26.4 kDa) and the Fd-DNAse I fusion (about 56.6 kDa), a third major band is observed at around 40 kDa. Interestingly, this band is observed in the humanised HMFG-1 fusions but not in the antibody alone. Since an anti-F(ab′)₂ antibody was used for immuno-precipitation, it is unlikely that this can be proteolysis between immunoglobulin and DNase I sequence. It probably represents a population of polypeptide produced by premature transcriptional termination (due to DNase I sequence in the 3′-end of the fusion mRNA).

GEL B

This is the non-reducing gel counterpart to gel A, described above. Lane 1 is the mock-transfected control cells and lanes 2 and 3 are from the cells transfected with humanised HMFG1 alone (construct 6) and the humanised HMFG-1 fused at the C-terminus to human DNase I, respectively. As before, lanes 4 to 6 are from cell supernatants from cells transfected with constructs 35, 36 and 39. The gel shows that both the whole antibody and the antibody-DNase I fusion are assembled, with the DNase fusion giving a higher molecular weight compared to the antibody alone.

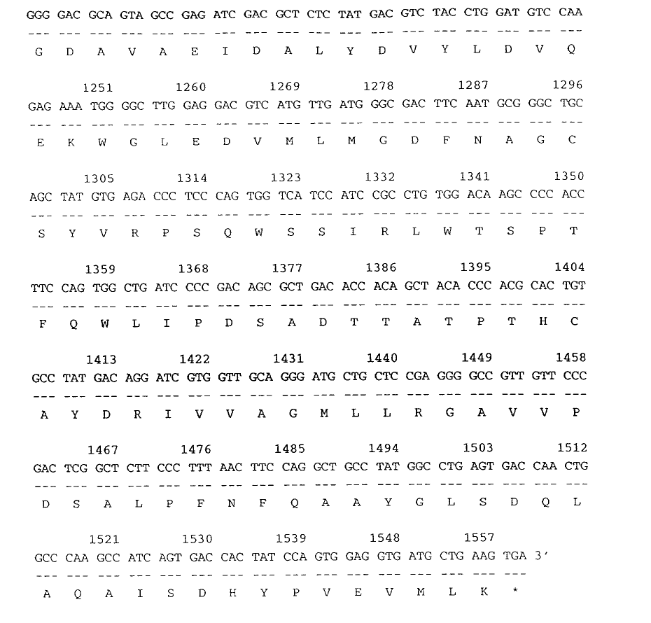

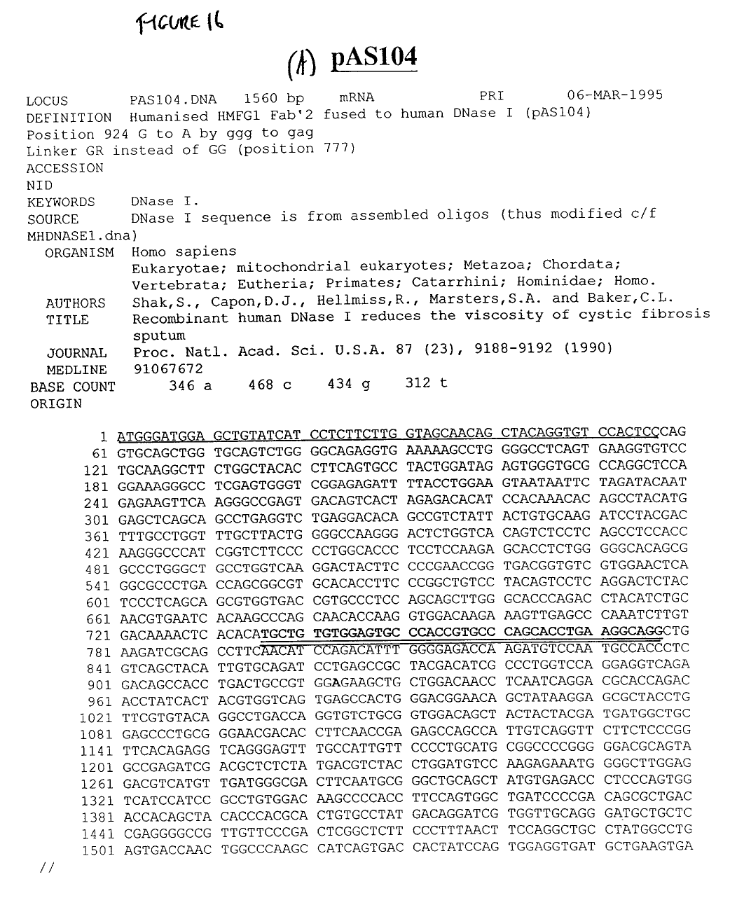

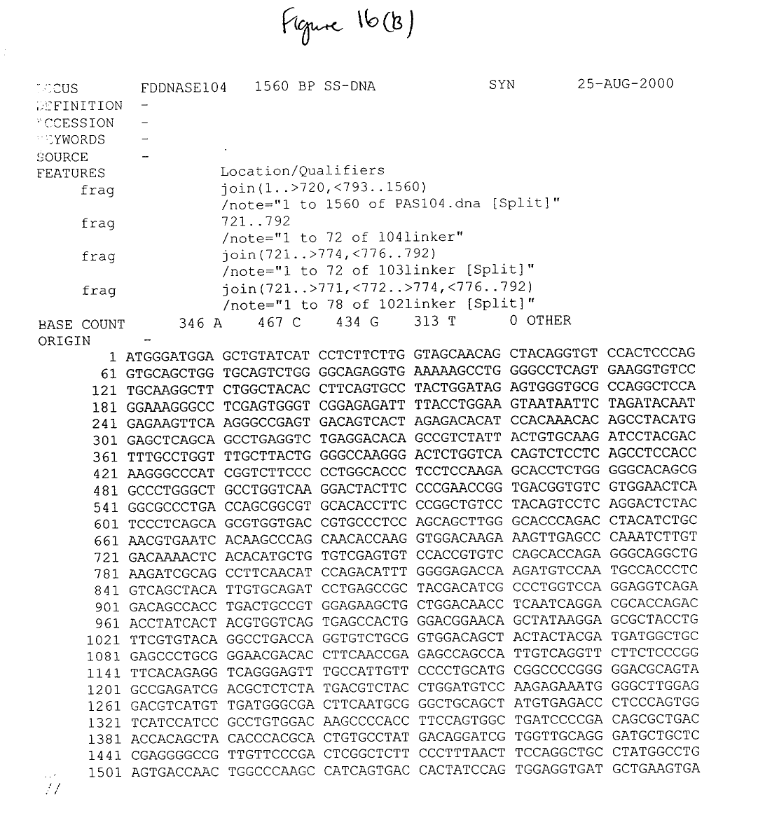

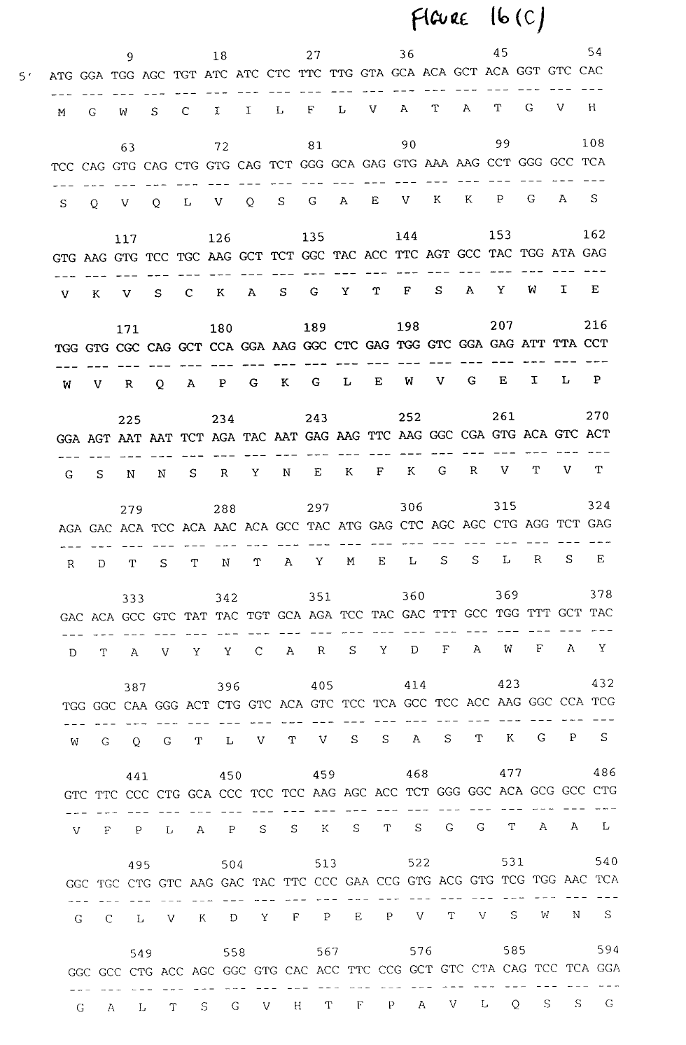

FIG. 22 shows a typical standard curve used to determine the concentration of PDTRP-binding material in the supernatants of transiently transfected L761h cells. Each point on the curve has been determined twice.

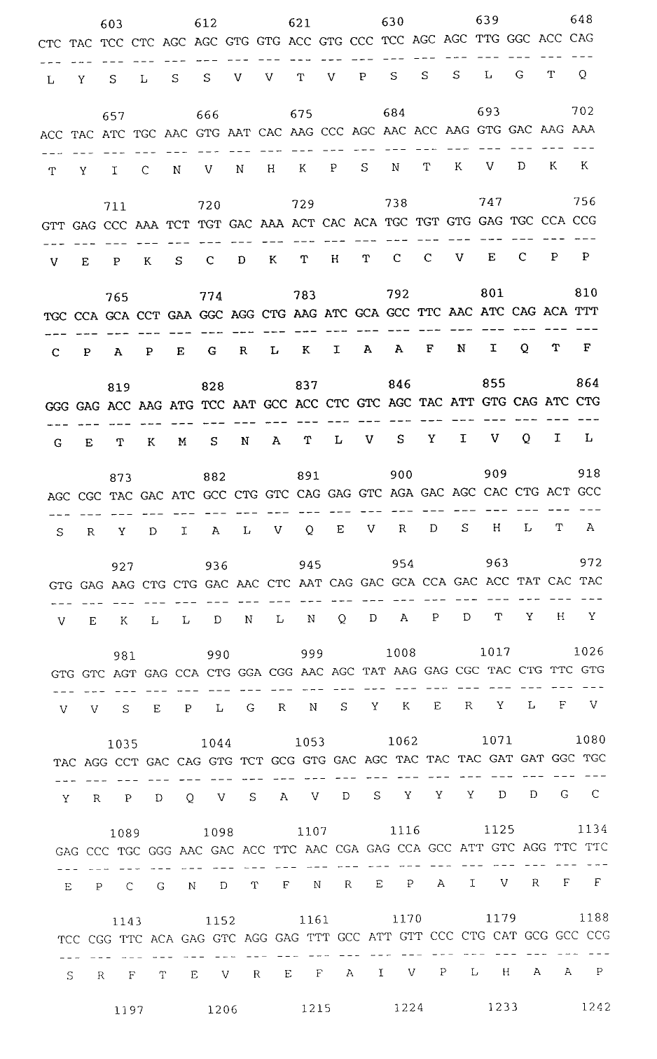

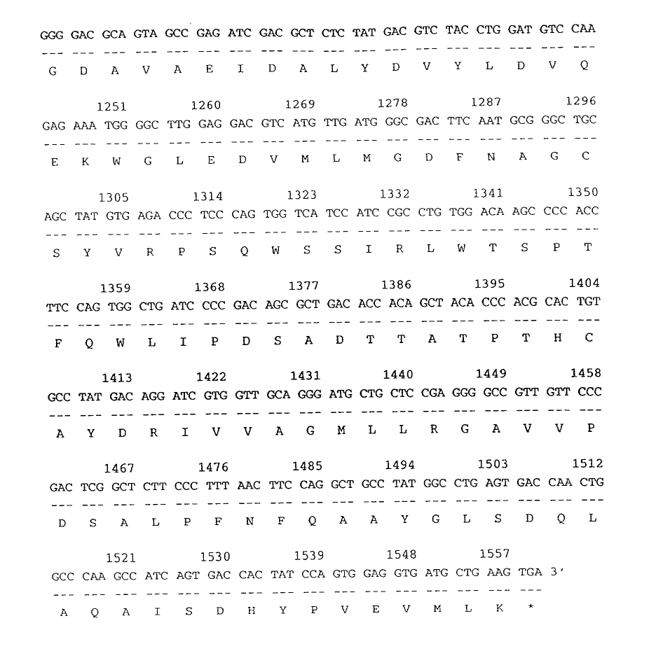

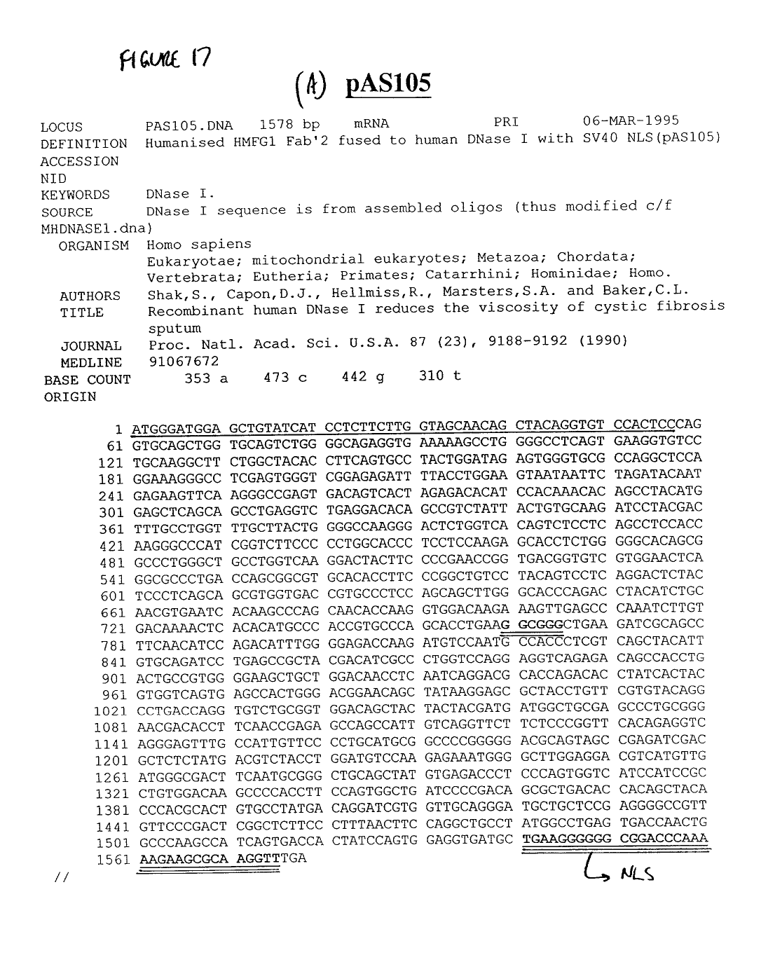

FIG. 23 shows typical standard curves used to determine the concentration of bovine DNAse I.

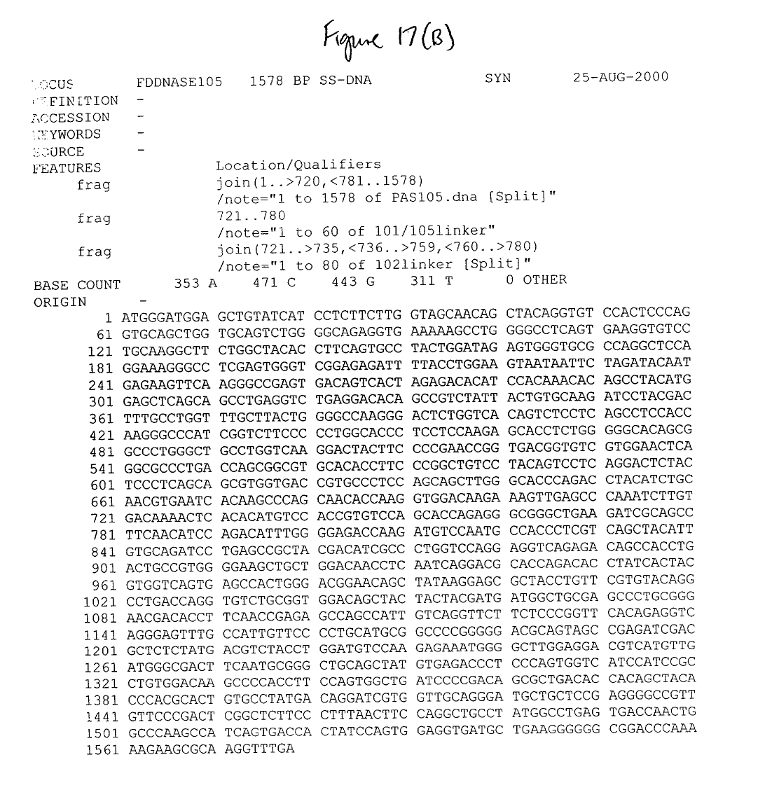

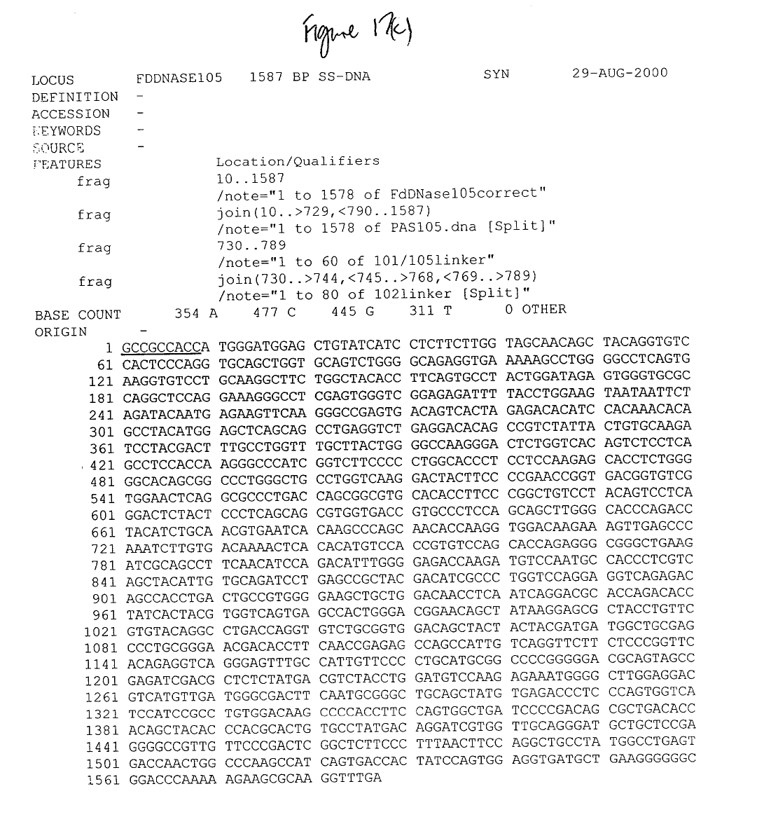

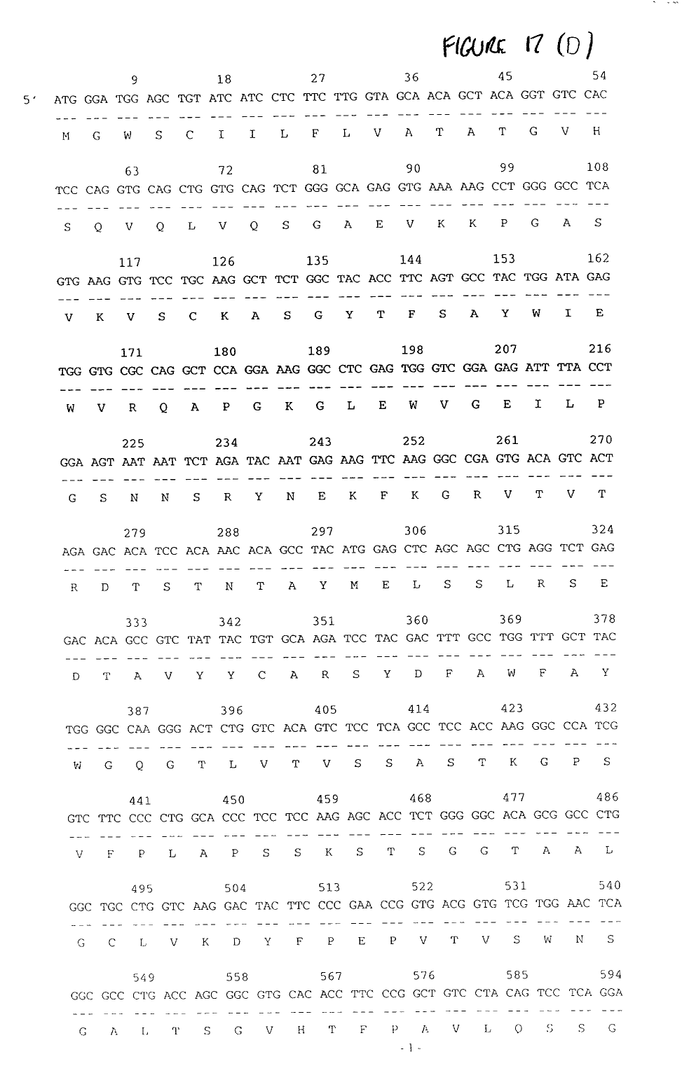

FIG. 24 shows corrected DNase I activity in transiently expressed humanised HMFGl whole antibody-human DNAse I fusions (i.e. pAS34, pAS34, pAS35 and pAS6[control]).

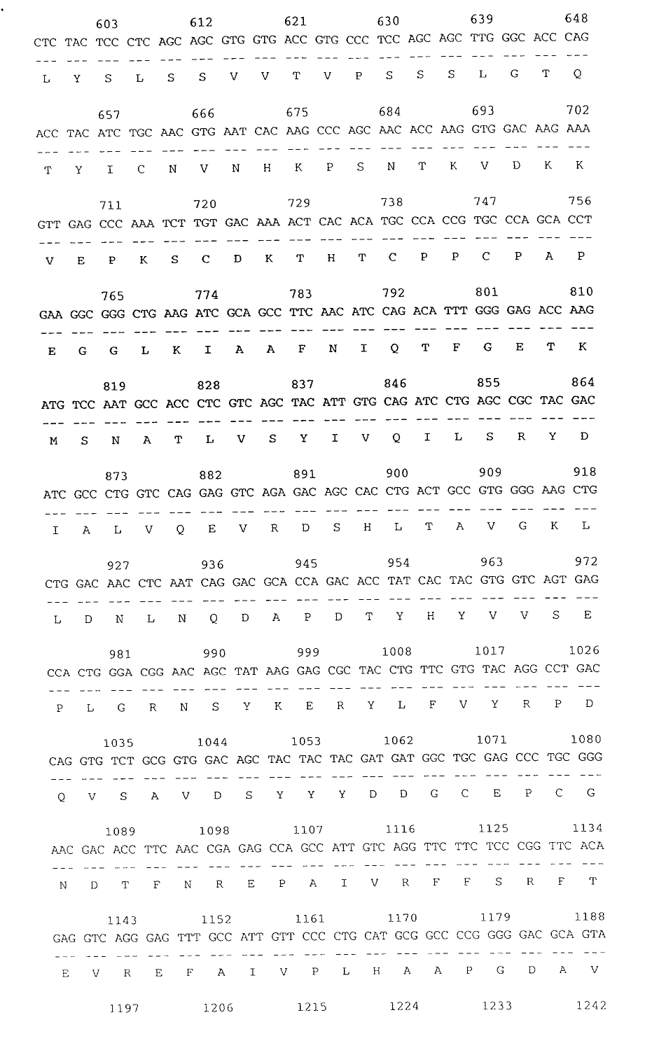

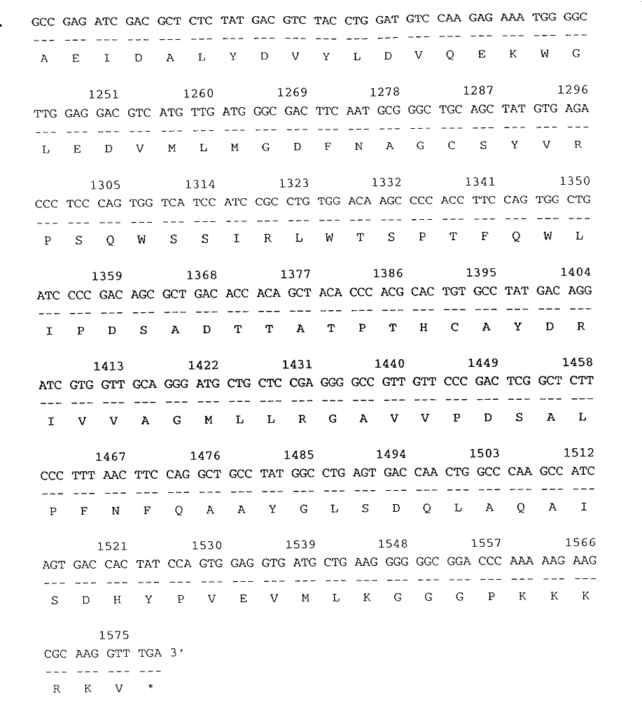

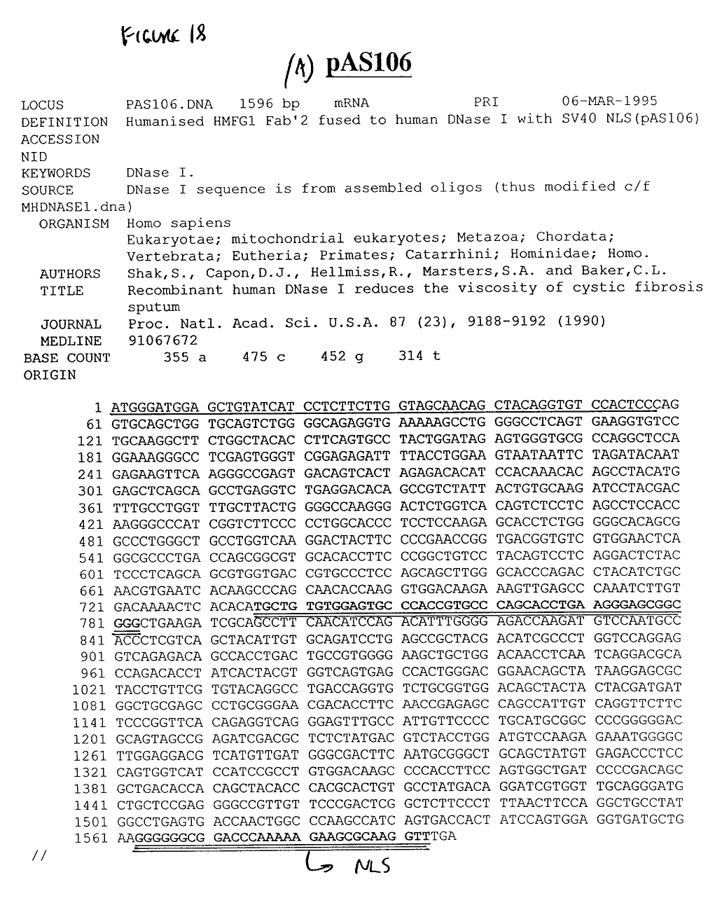

FIG. 25 shows the corrected DNAse I activity in transiently expressed humanised HMFG1 F(ab′)₂-human DNase I fusions (i.e. pAS101, pAS 102, pAS 103 and pAS41 [control]).

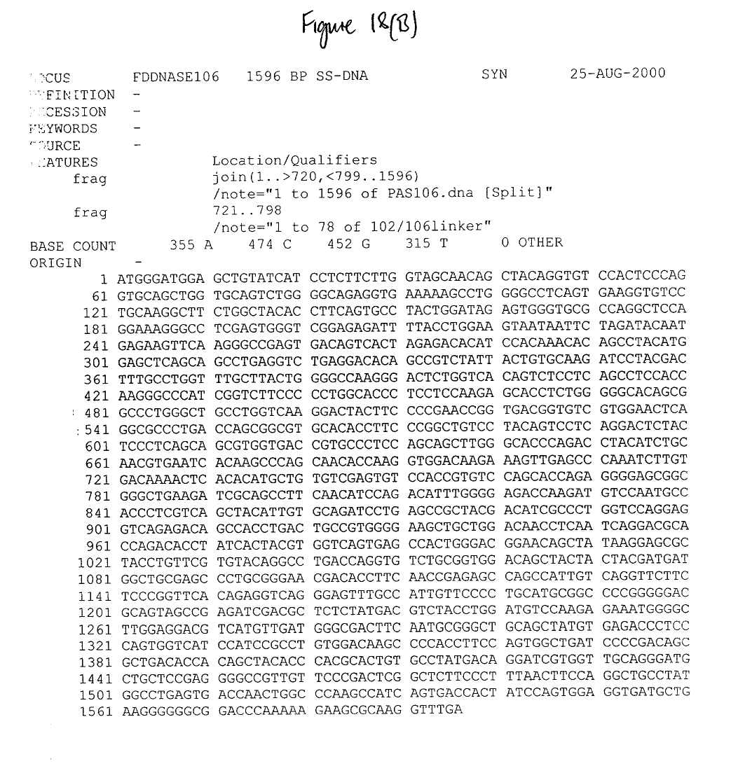

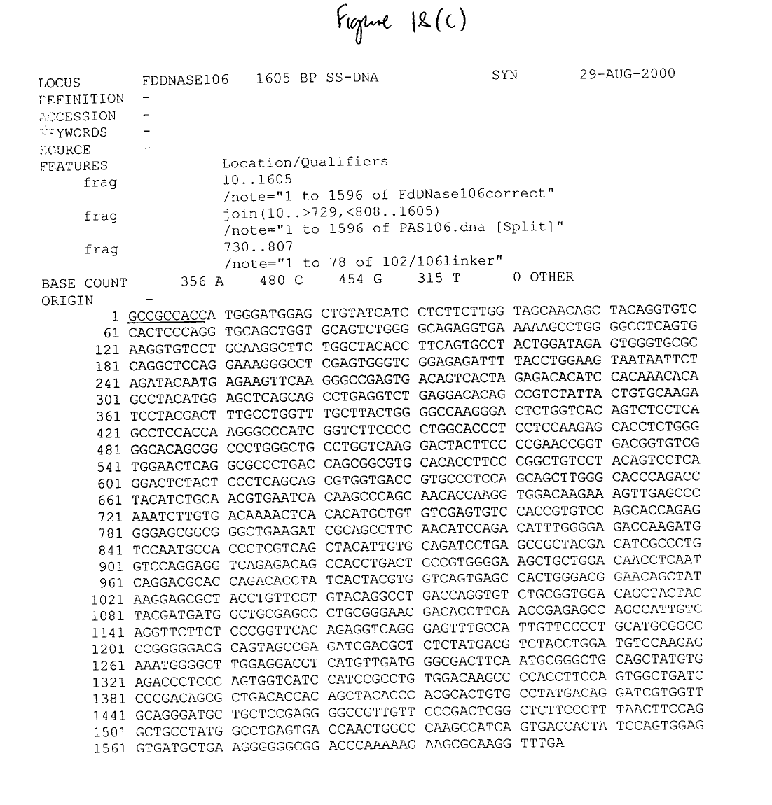

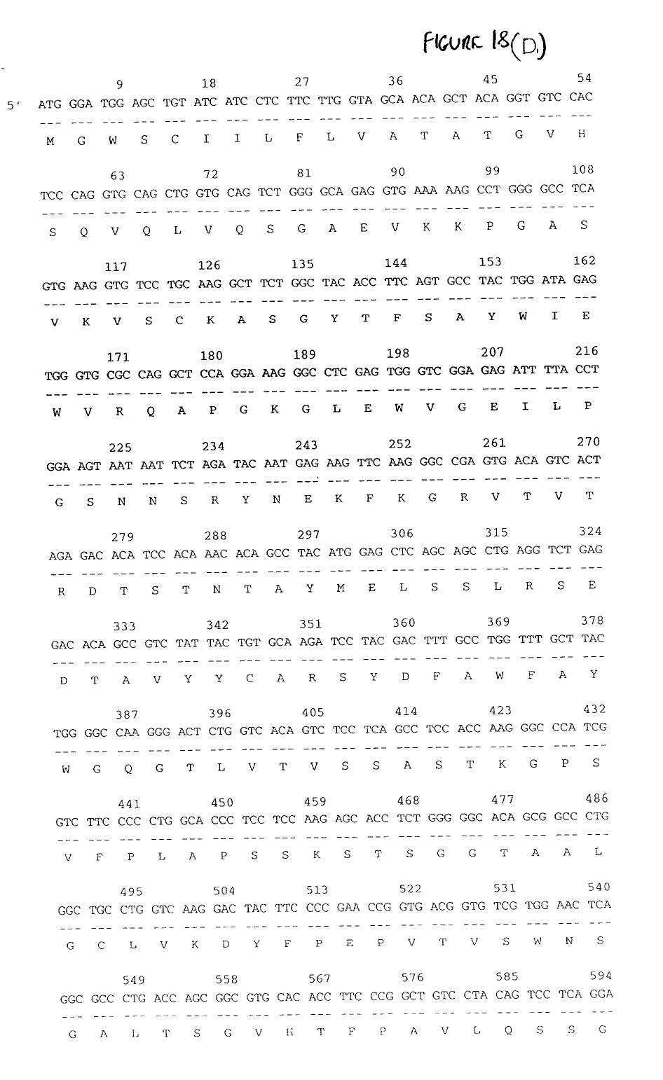

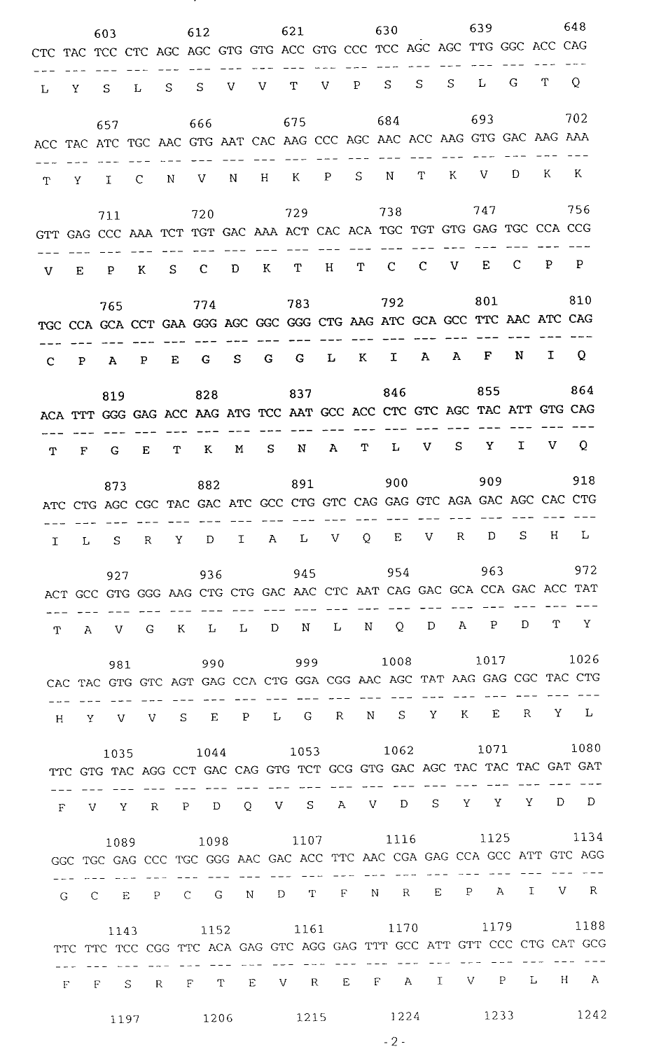

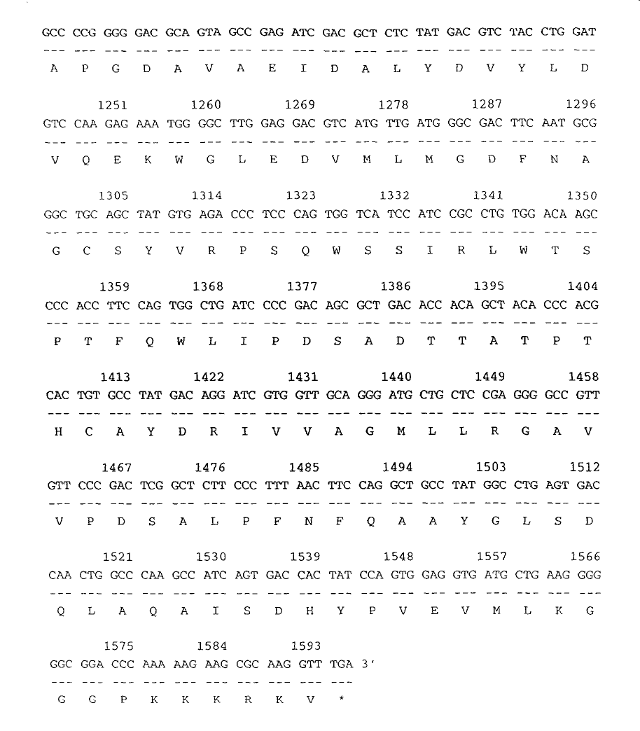

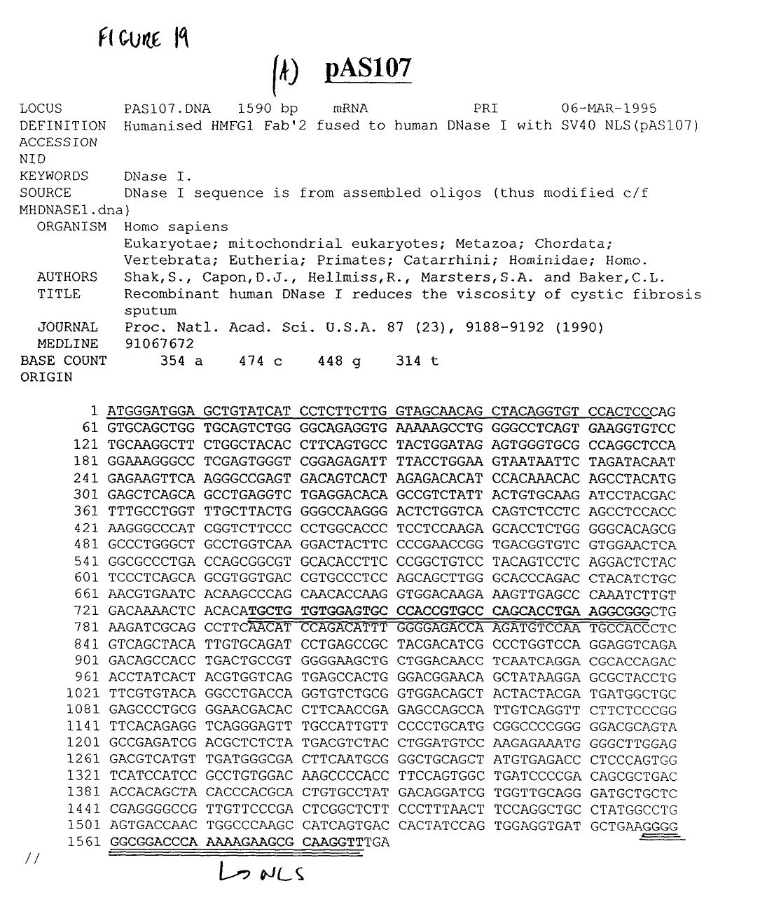

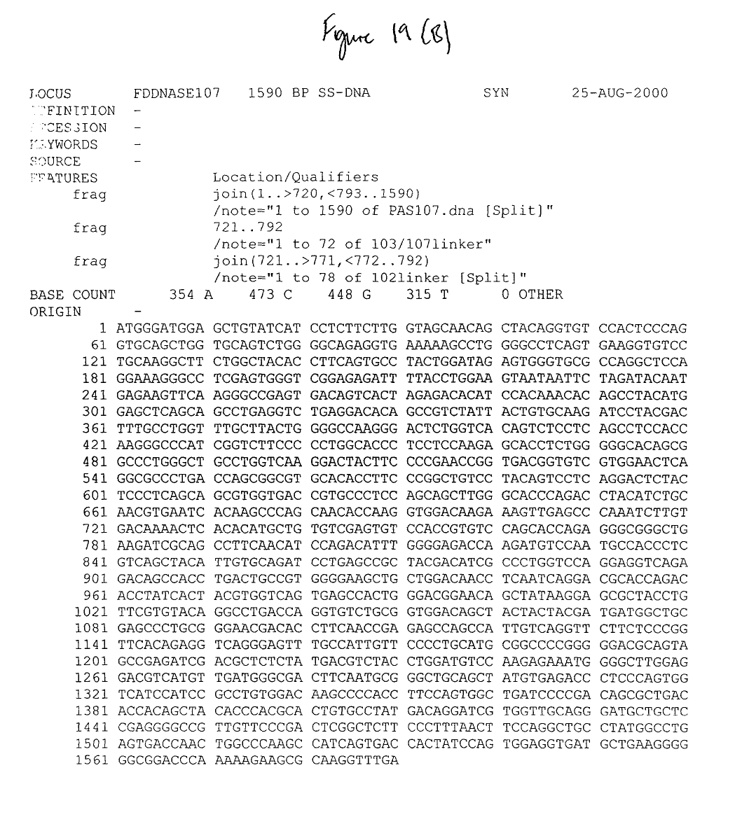

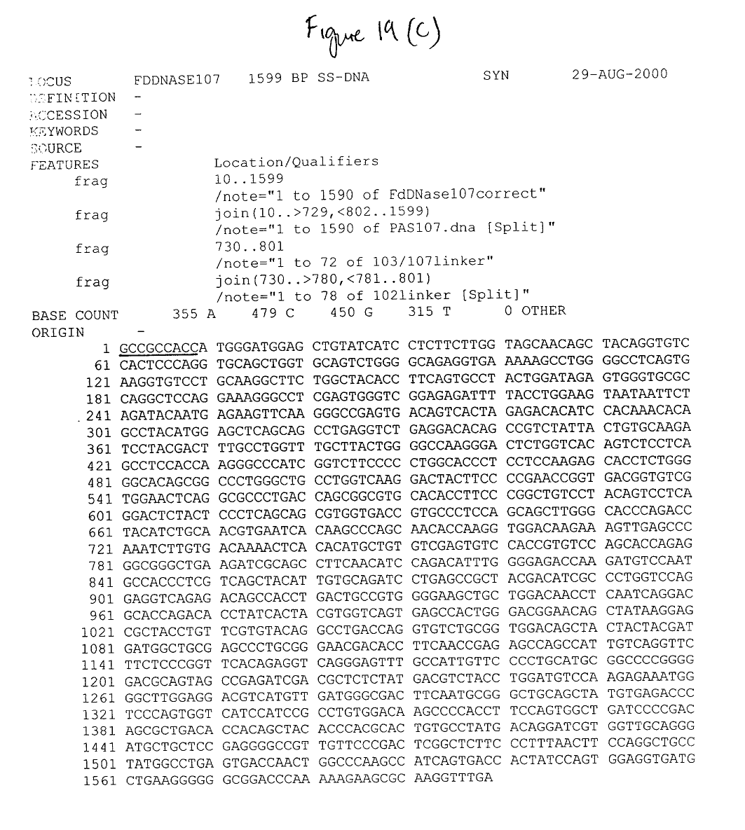

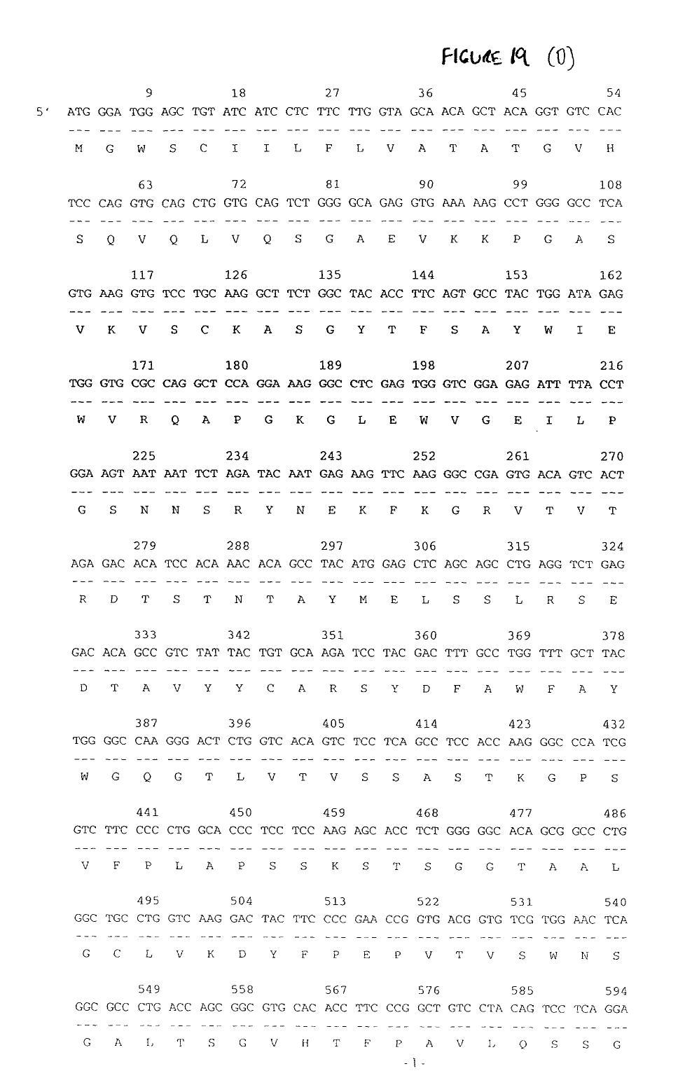

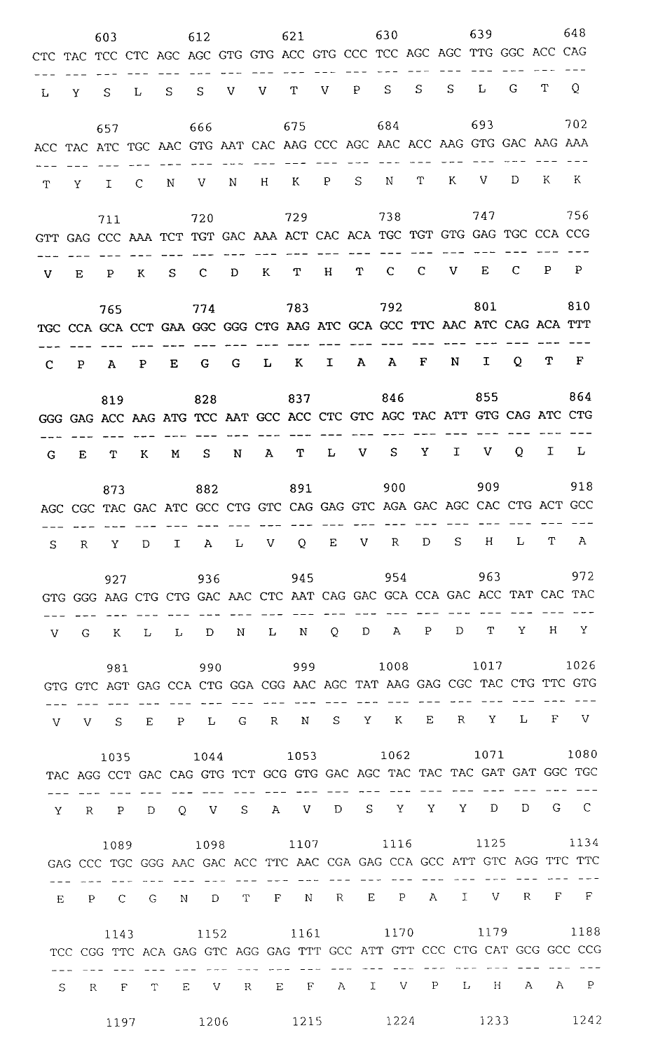

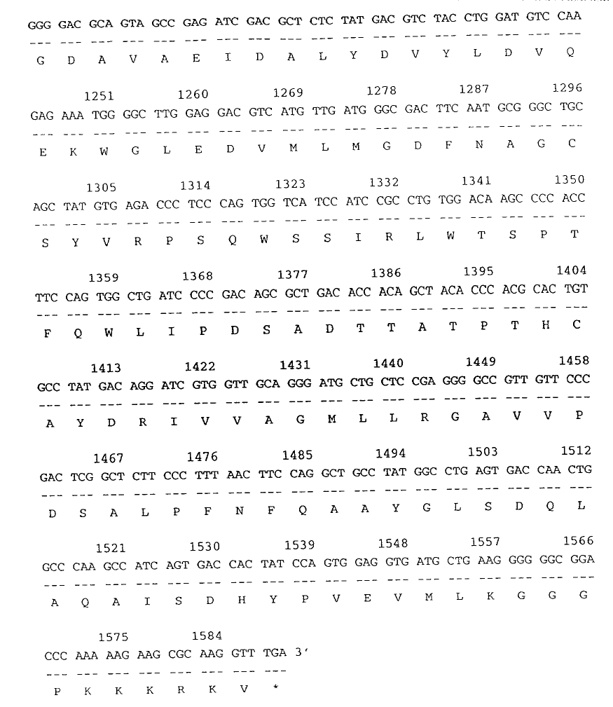

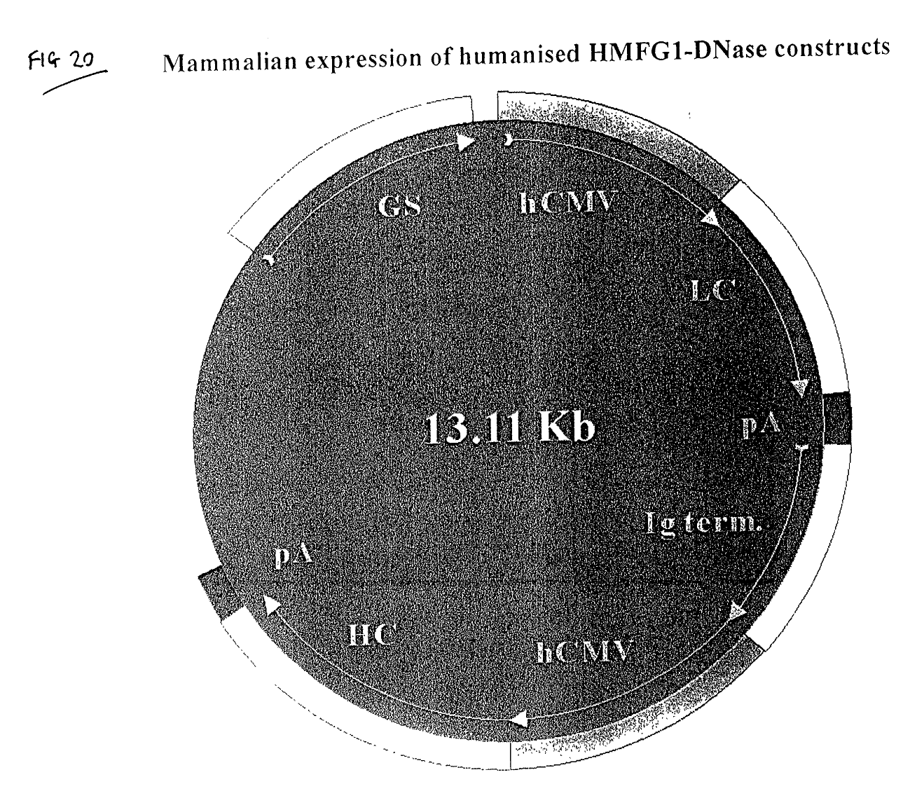

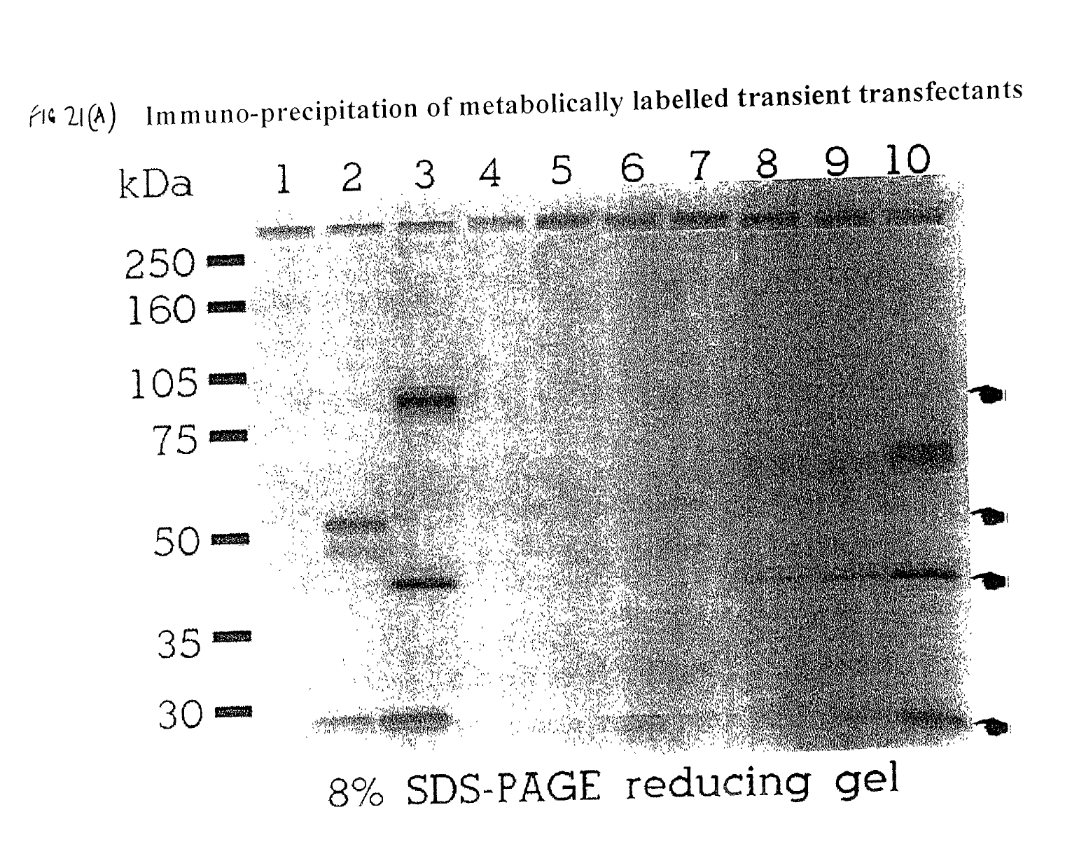

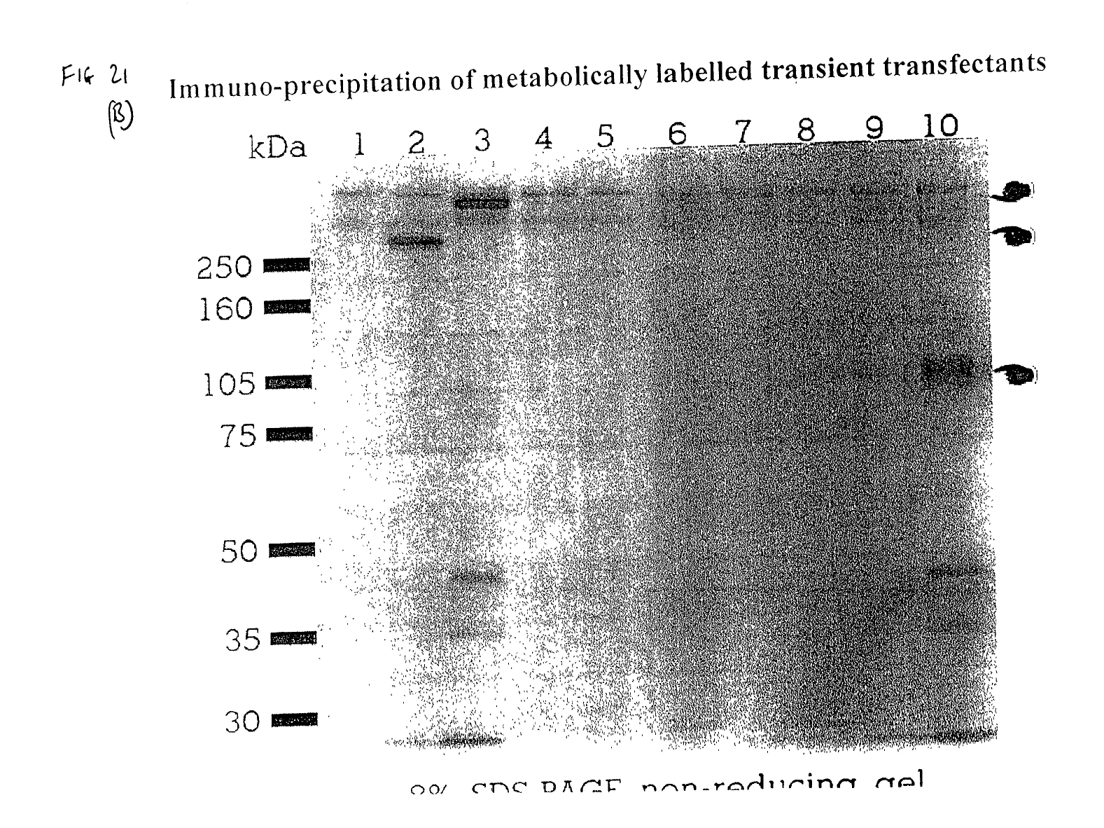

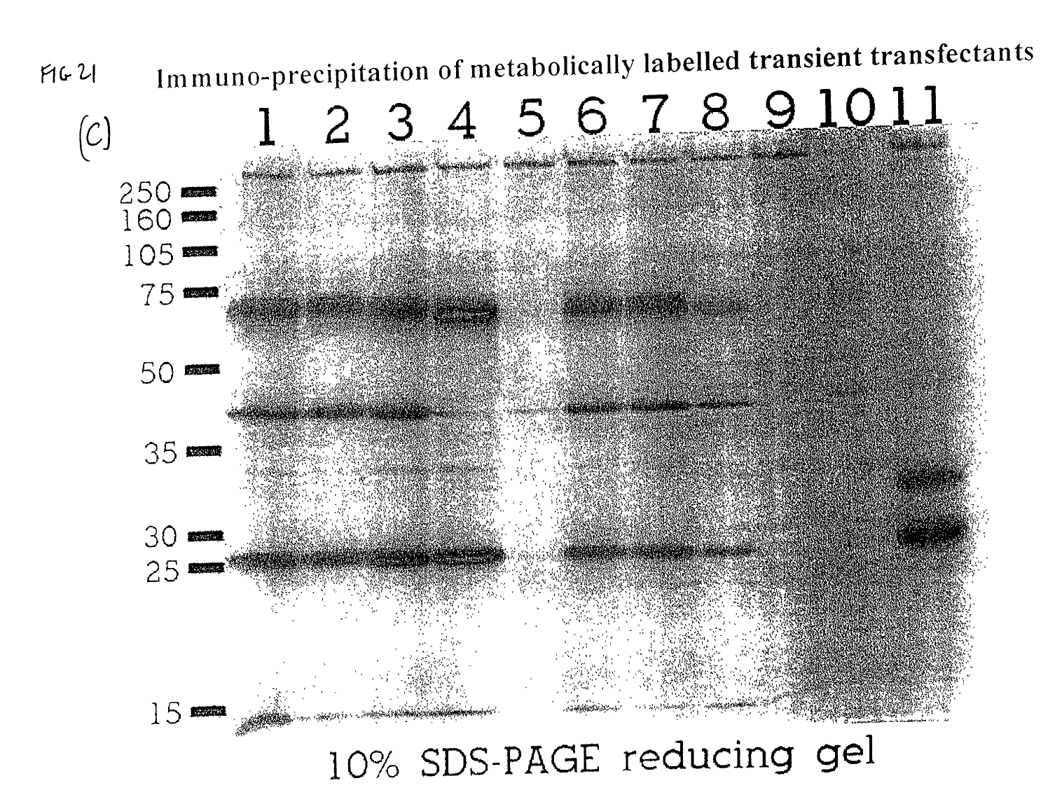

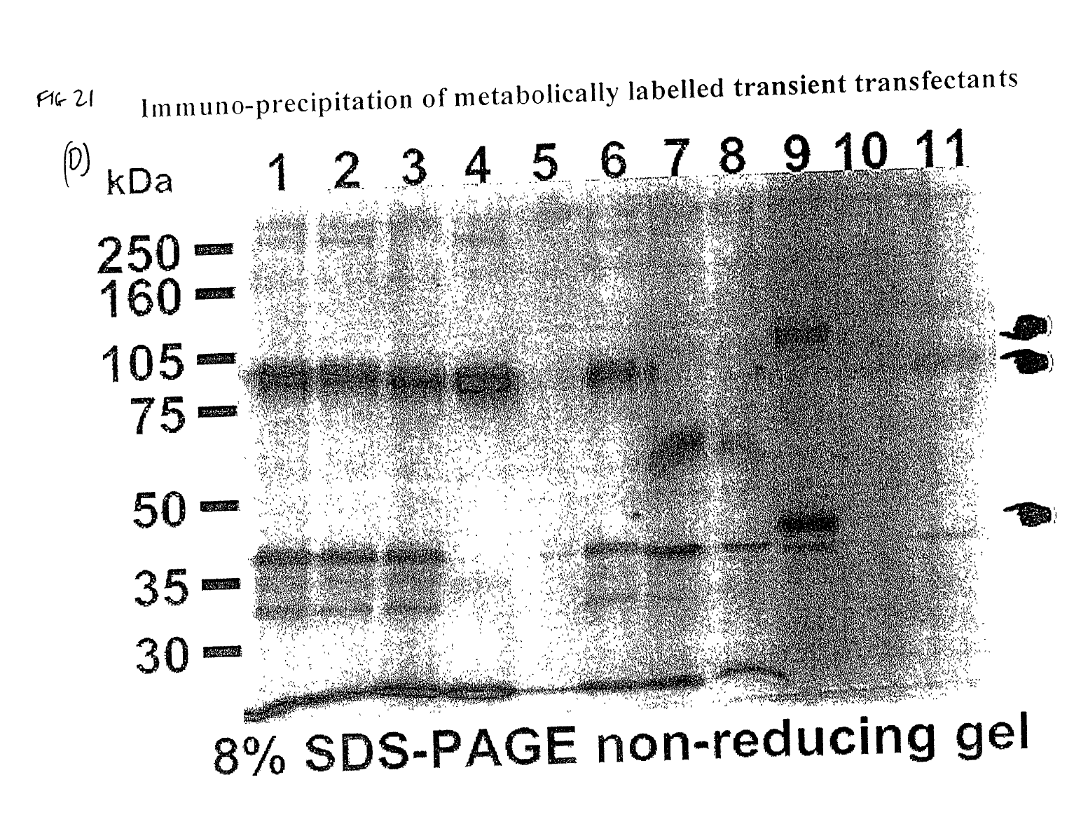

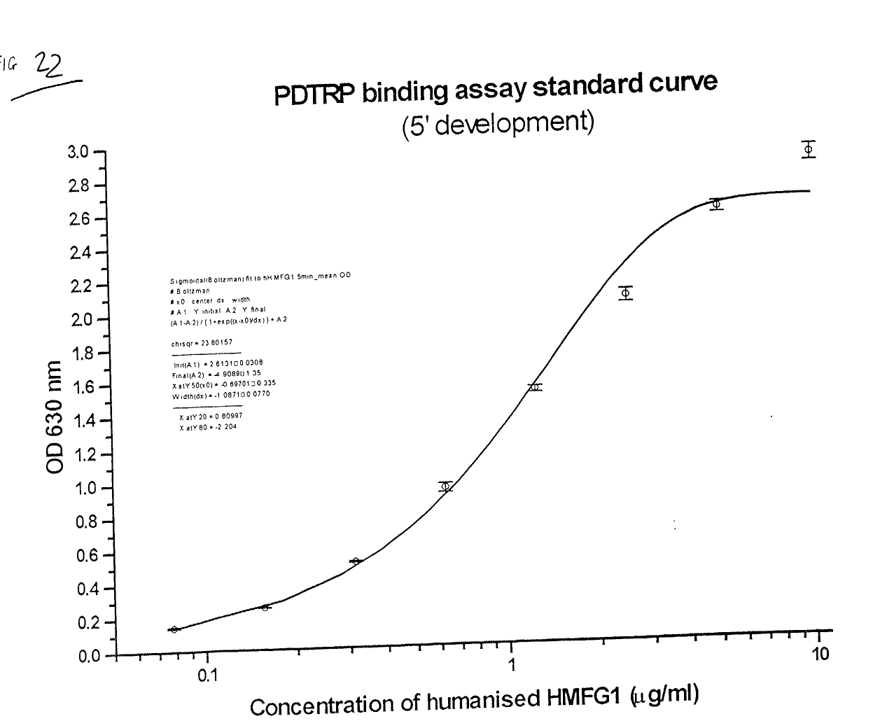

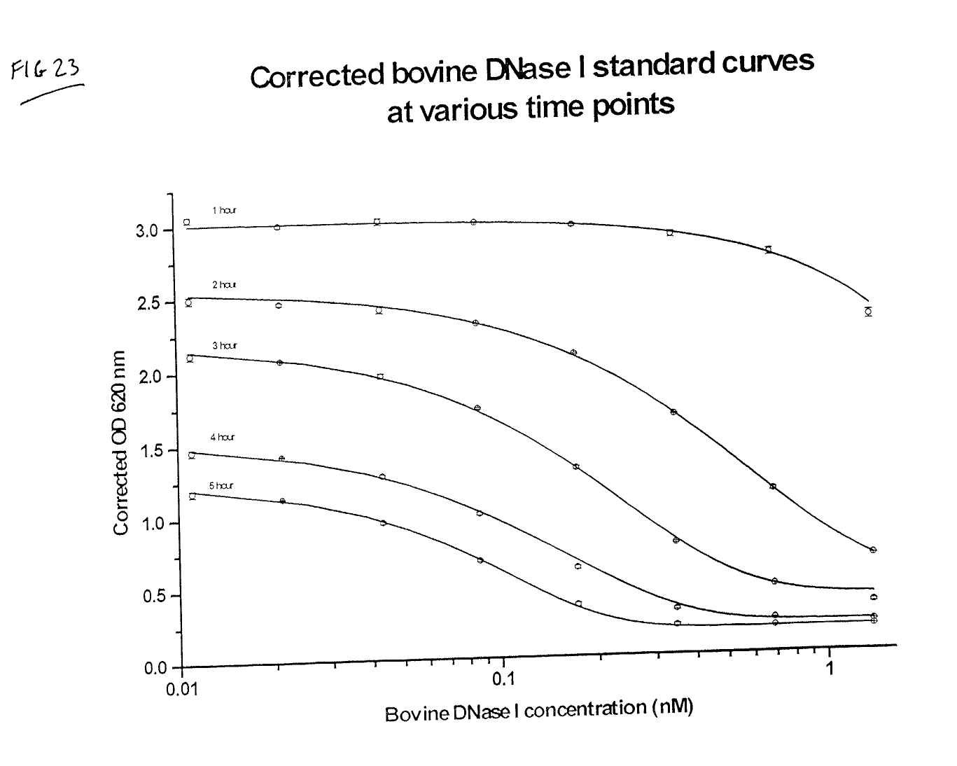

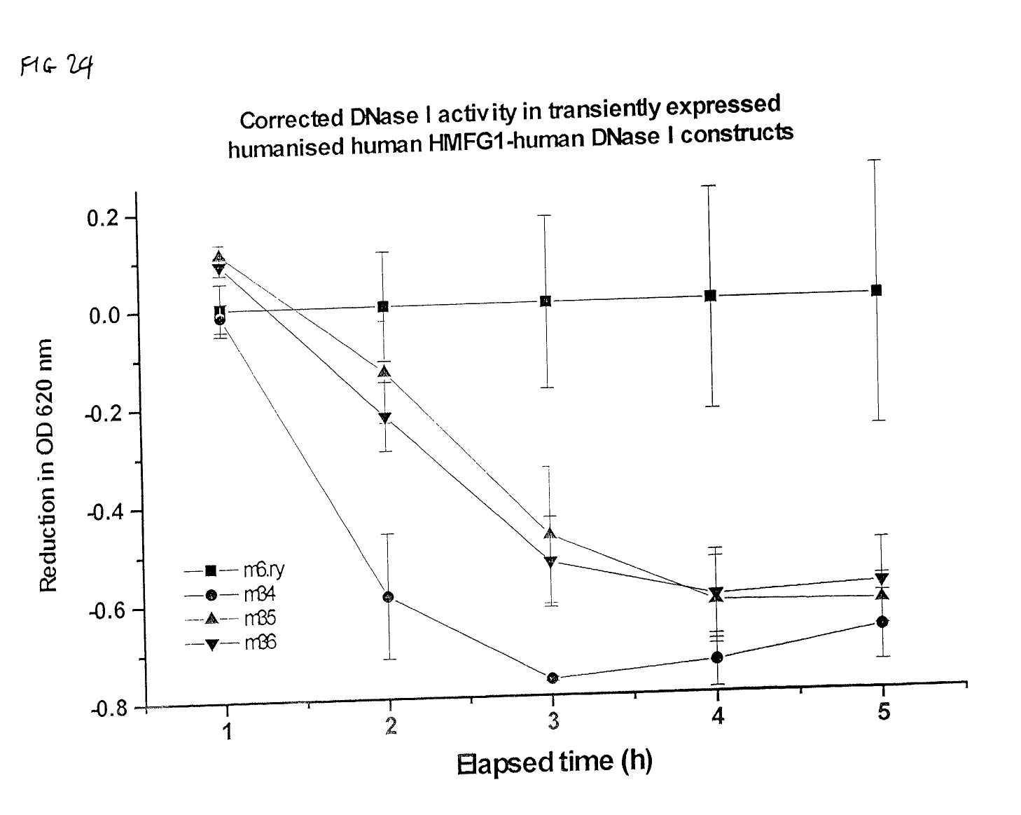

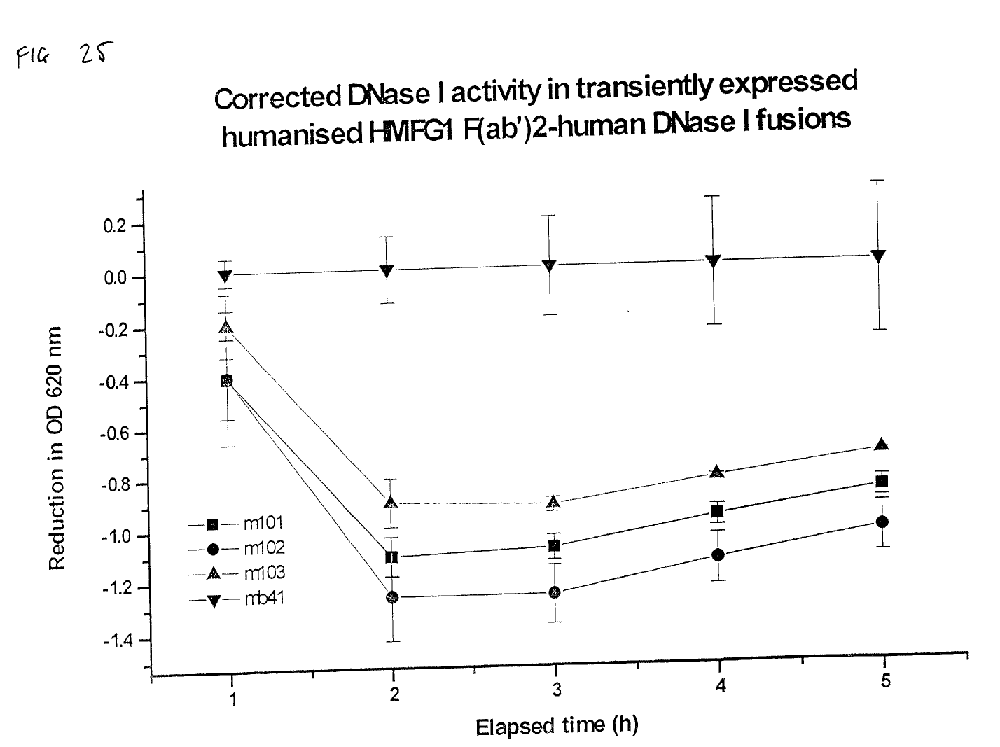

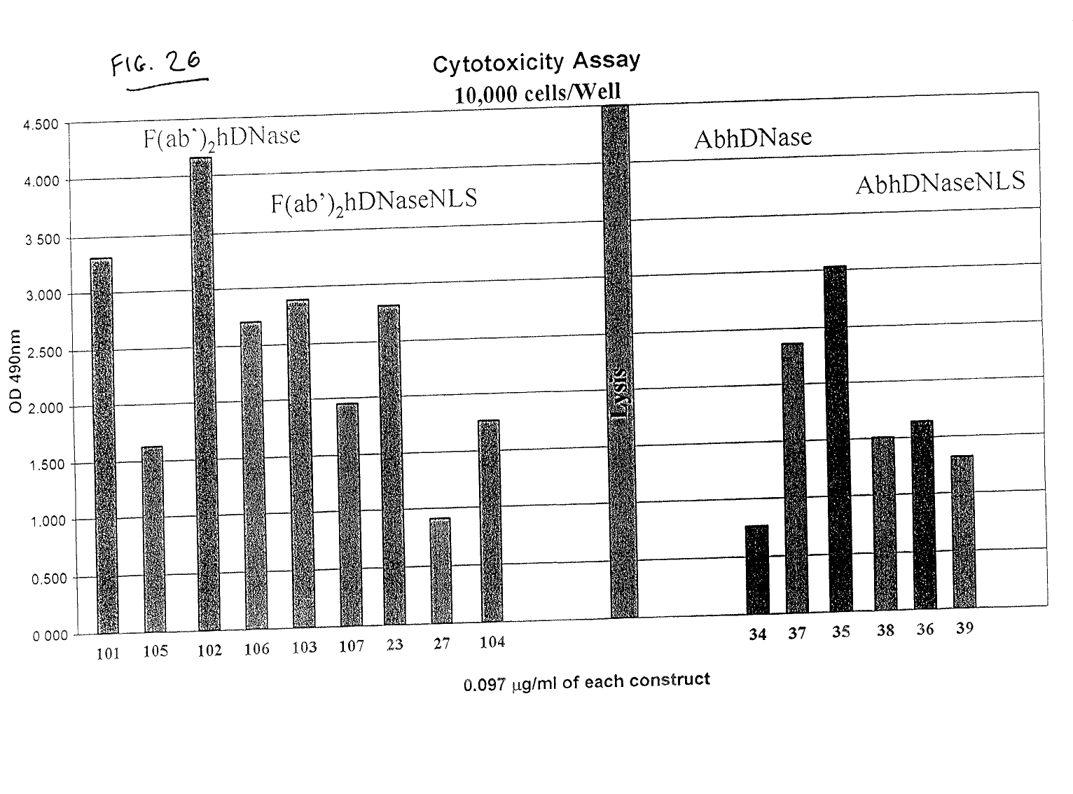

FIG. 26 shows results of the cytotoxicity assay.

FIG. 27 shows the % of MCF7 cells killed after incubation with the exemplary constructs.

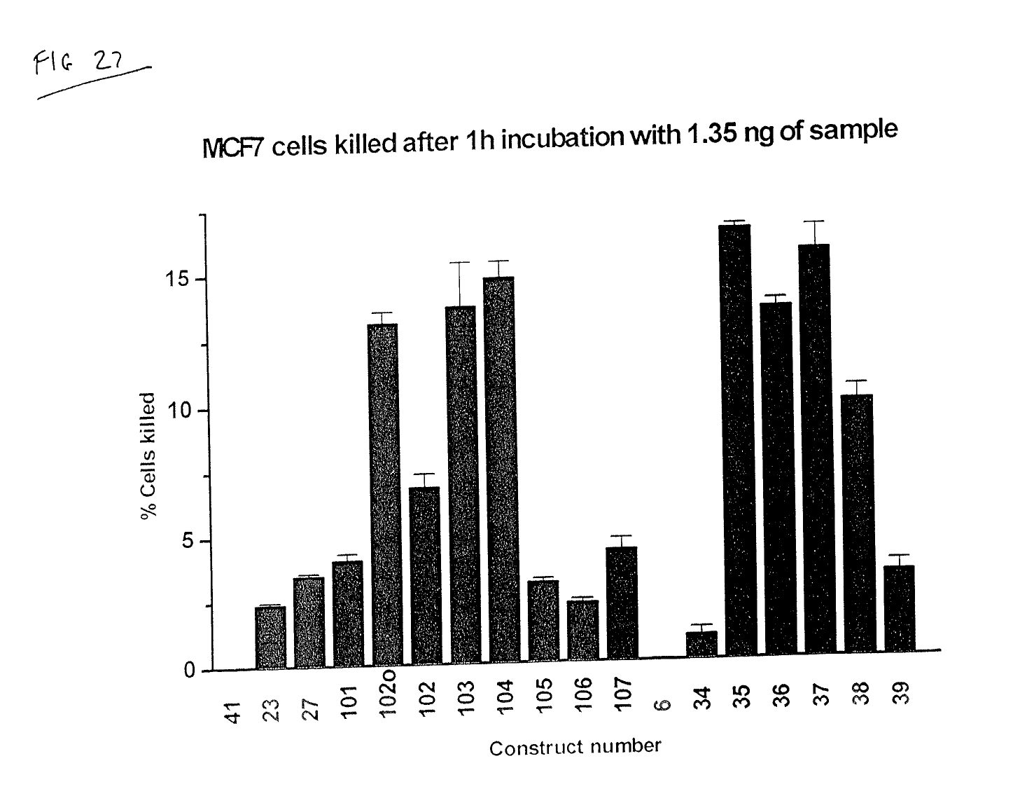

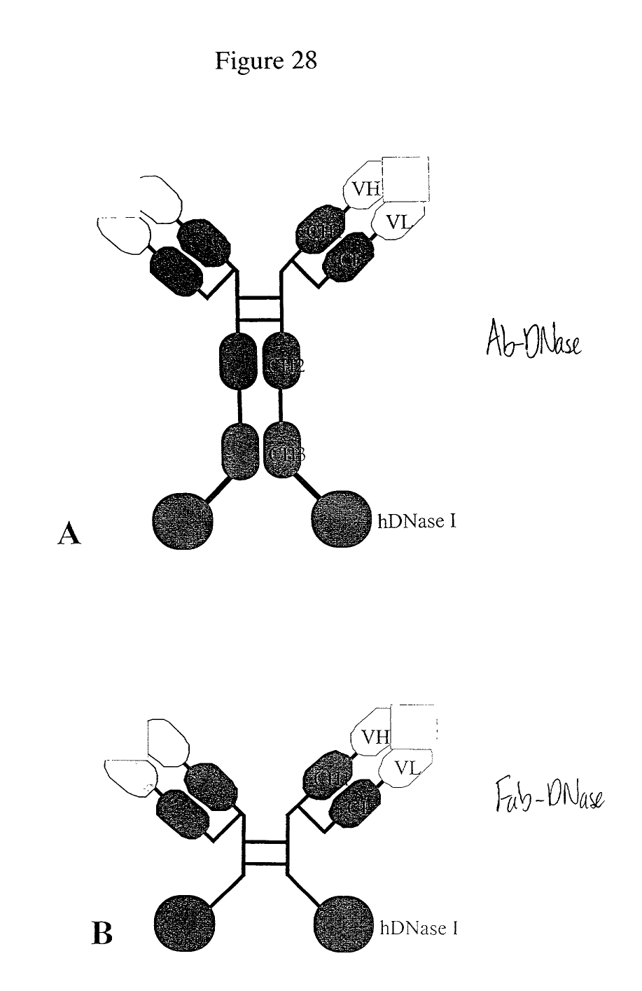

FIG. 28 shows a schematic diagram of (A) Ab-DNase and (B) Fab-DNase.

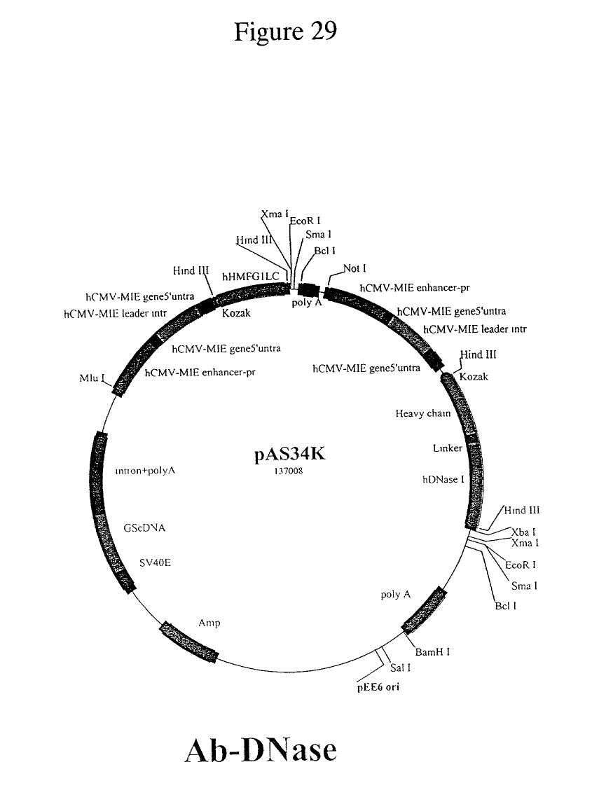

FIG. 29 shows a schematic diagram of vector pAS34K encoding Ab-DNase (i.e. pAS34 as shown in FIG. 7b plus Kozak sequence).

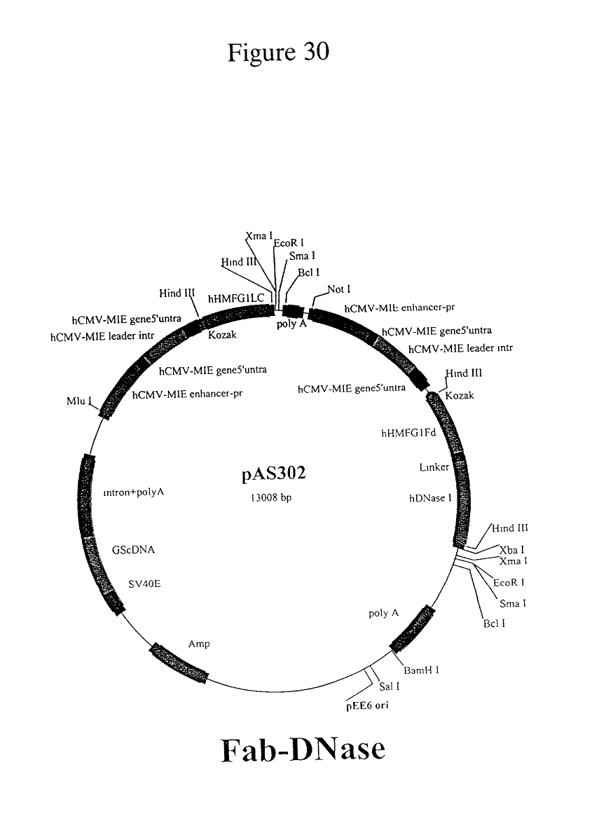

FIG. 30 shows a schematic diagram of vector pAS302 encoding Fab-DNase.

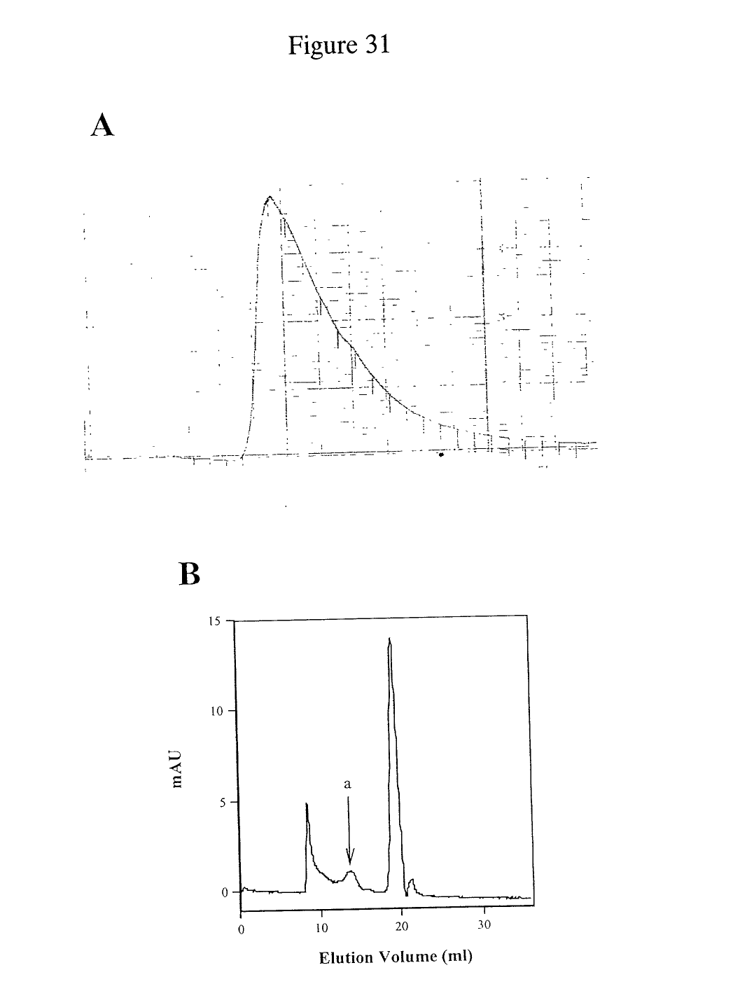

FIG. 31 shows (A) the elution profile from Protein-L column and (B) size exclusion chromatogram for Fab-DNase.

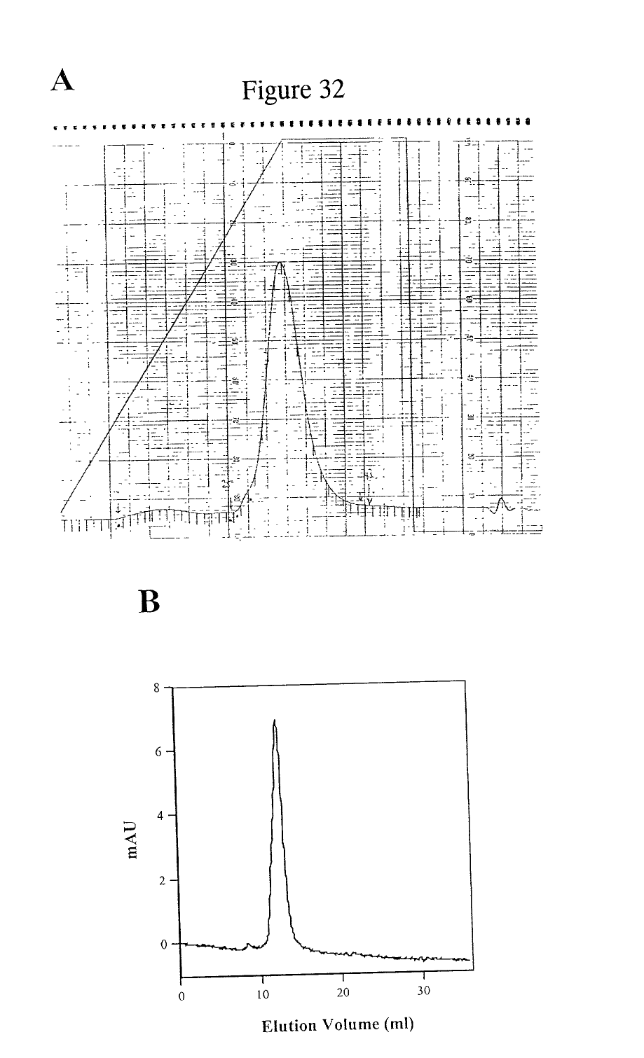

FIG. 32 shows (A) the elution profile from Protein-A column and (B) size exclusion chromatogram for Ab-DNase.

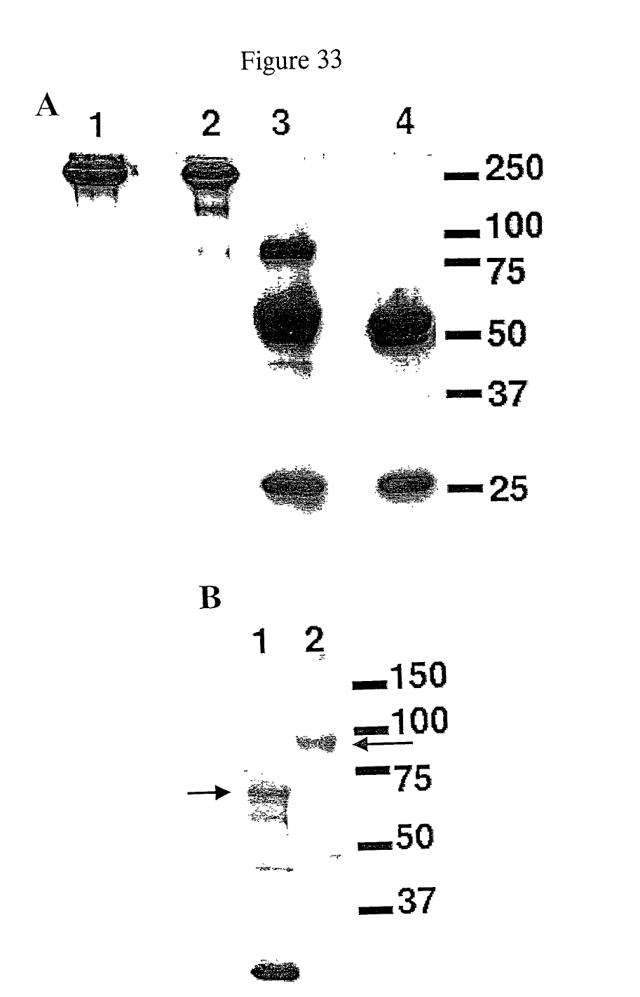

FIG. 33 shows the SDS-PAGE stained gels for (A) Ab-DNase and (B) Fab-DNase.

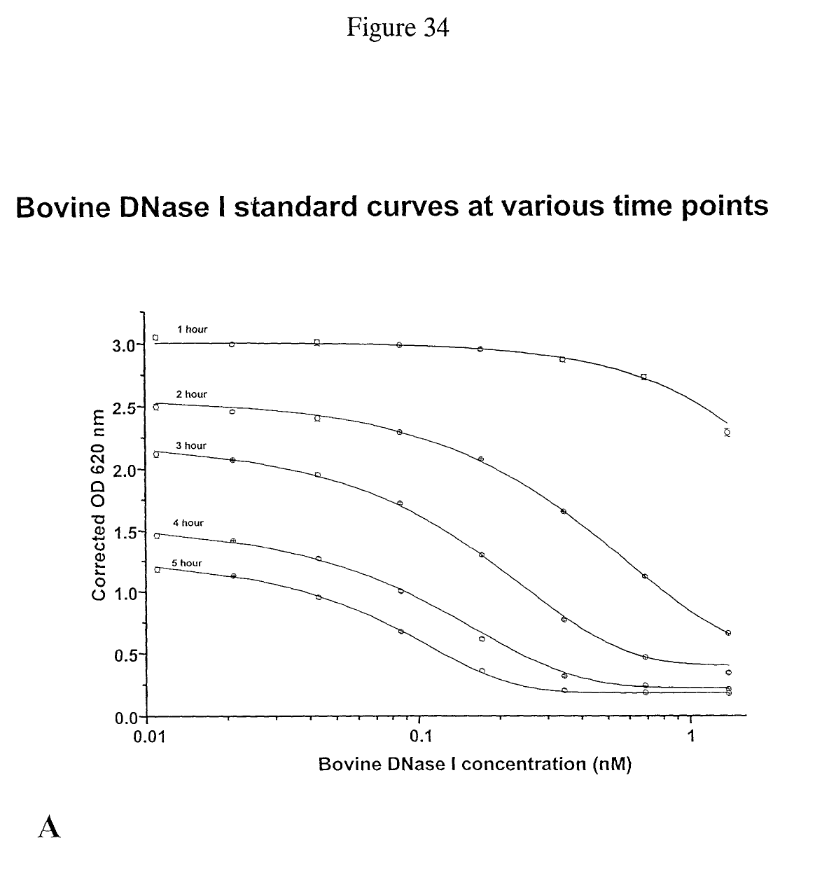

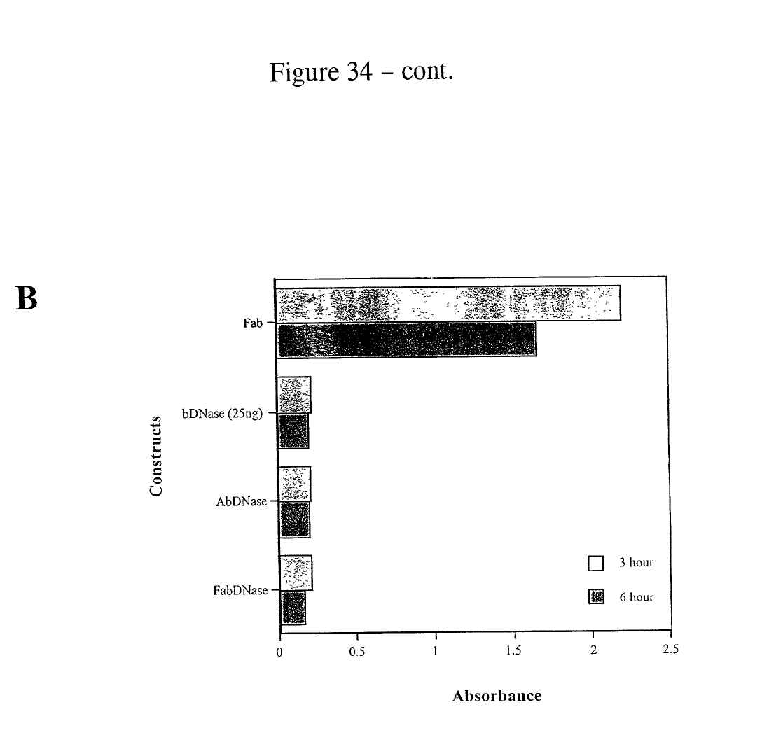

FIG. 34 shows (A) standard curve for bovine DNase concentration AND (B) DNase activity measurements at 3 hours and 6 hours.

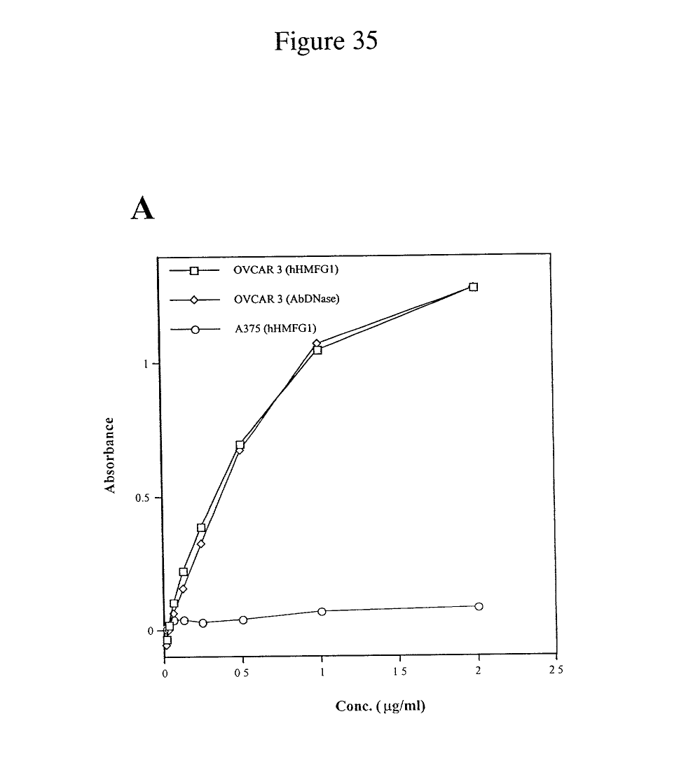

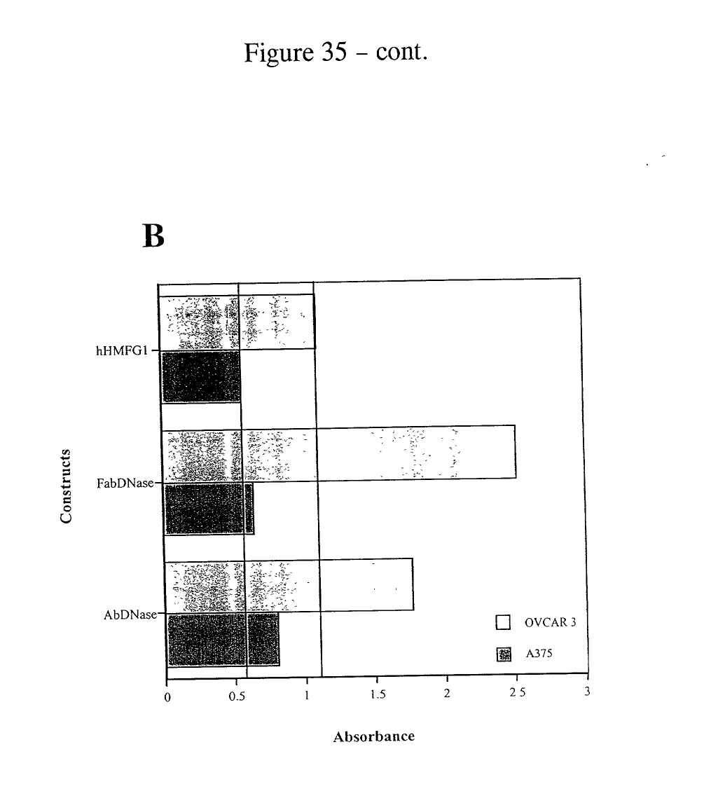

FIG. 35 shows (A) PEM expression on OVCAR 3 and A375 cells, as measured by ELISA using hHMFG-1 and AD-DNase antibodies, and (B) cytotoxicity measurements.

### EXAMPLES

### Example 1

(A) Mammalian Expression of Humanised HMFG1-DNase Constructs

The human HMFG1 light and heavy chain (with or without engineering a fusion to human DNase I), were cloned into the pEE6 expression vector system for expression in mammalian CHO or myeloid NS0 cells (see FIG. 20). The vector system was originally developed by Celltech Ltd (UK) and is now owned by al-Lonza (see Young & Owens, 1994, J. Immunol. Meth. 168:149-165). The vector consists of two human cytomegalovirus promoters (hCMV) for both the heavy and light chain genes. Each transcription unit is completed by the poly-adenylation signal (pA) with an optional immunoglobulin terminator sequence (Ig term.) located between the heavy and light chain transcription units. Propagation in E. coli can be selected for by the presence on an ampicillin resistance gene (not shown in FIG. 20). The inclusion of a glutamnine synthetase gene (GS) in the vector allows the stable NS0 transfectomas to be selected by growth in glutamine free media, since NSO cells are GS⁻ and cannot otherwise grow in glutamine free media.

Exemplary humanized HMFG1-DNAse I fusion constructs of the invention are detailed in FIGS. 5 to 19.

(B) Immuno-precipitation of Metabolically Labelled Transient Transfectants

CHO-L761h cells (Cockett et al., 1990, Nuc. Acids Res. 19:319-325) were transfected, according to the modification of Gorman et al, 1985), with expression vectors containing either whole HMFG1 antibody or F(ab′)₂ fragment of the antibody along with the various fusion constructs of their respective heavy chains and human DNase I. The cells were then incubated with either 50 μCi ³⁵S methione for 72 h in methionine-free medium. Secreted product was precipitated with a rabbit anti-human F(ab′)₂ antibody bound to protein A Sepharose. Bound material was eluted in either reducing or non-reducing SDS-PAGE loading buffer and run on gels. The autoradiographs (see FIG. 21) above were generated from those gels after drying them.

(C) Estimation of the Efficiency of DNase Constructs in Supernatants

Introduction

This set of experiments was designed to standardise the amount of construct in a given DNase I activity assay and to allow us to comment on the amount of activity a particular construct possesses. Given that the antibody-DNase I fusions are so different to the F(ab′)₂-DNase I fusions it is best not to compare the two groups. Once we have purified the protein, we will have a better idea of the exact molecular configuration of all species. Then, and only then, will it be sensible to compare amongst groups.

Determination of Concentration of Constructs

The concentration of constructs in supernatants from transiently transfected L761H cells was determined in a PDTRP-binding ELISA. To each well of a Maxisorb 96-well ELISA plate (Nunc) was added 100 μl of carbonate buffer containing 100 ng of recombinant GST-(PDTRP)₇ fusion protein (Gendler et al., 1990, J. Mol. Biol. 265:15286-93). After overnight binding at 4° C., the plate was washed three times in PBS-Tween (i.e. PBS containing 0.05% Tween-20). The plate was then blocked with three 3-minute washes of PBS-Tween containing 1% BSA.

For each construct, 100 μl of supernatant was added to a well on the plate. In addition, hHMFG-1 of known concentration was serially diluted down the plate using doubling dilutions in 100 μl of PBS-Tween per well. The plate was incubated for a further 1 h at 30° C., then 200 ng of MC 135 anti-human kappa light chain antibody (binding site) in 100 μl of PBS-Tween was added to each well for 1 h at 30° C. After three 3-minute washes in PBS-Tween, 100 μl of anti-mouse IgG-peroxidase conjugate (Jackson 315-035-045), diluted 1:2000 in PBS-Tween, was added to each well and incubated for 1 h at 30° C. Following a final set of three 3-minute washes in PBS-Tween, 100 μl of TMB substrate (Sigma) was added to each well of the plate and, after a colour developed, the optical density at 630 nm of the solution in each well of the plate was determined.

Results

(see FIG. 22)

(D) Corrected Bovine DNase I Standard Curves and DNase Assay

DNase activity was determined using a modification of the methyl green-DNA complex degradation method (Sinicropi et al., 1994, Analyt. Biochem. 222:351-358). Briefly, a 1:1 solution of the assay buffer and methyl green-salmon sperm DNA complex was mixed together to give a total volume of 0.2 ml. To this, 0.1 ml of tissue culture supernatant from transiently transfected CHO-L761h cells was added and the mixture incubated at 37° C. DNA cleavage by DNase results in a reduction in absorbance at 620 nm. FIG. 23 shows a standard curve produced with various concentrations of bovine DNase I over a number a time point.

FIGS. 24 and 25 show DNAse activity for the whole HMFG1 antibody—and F(ab′)₂-DNase fusions, respectively.

(E) Cytotoxicity of DNAse Constructs

Method

DNase constructs were transfected into CHO L761h cells using a calcium phosphate co-precipitation method (Gorman et al., 1985, In: DNA cloning (2nd edition), Glover A(ed.), Academic Press, NY, 163-188). Included in the experiment were negative controls, consisting of cells transfected with TE buffer alone or with TE buffer and pEE6 expression vector. In addition to these controls, vectors that express hHMFG-l (pAS6) and F(ab′)₂ of hHMFG1 (both with specificity for PEM but without DNase I) were included.

The supernatant from these cells was harvested after 72 h of expression, followed by centrifugation to remove dead cells. MCF-7 cells were incubated for 1 h at 37° C. with an aliquot of each of these supernatants. The amount of cellular lactate dehydrogenase (LDH) released from the MCF-7 cells due to the cytotoxicity of the supernatant was determined using the CytoTox96 cytotoxic assay kit (Promega). Total lysis (‘total LDH’) was determined by measuring the target cell maximum LDH release using the kits lysis solution. The percentage of cells killed was then calculated as the proportion of the LDH released to the total LDH released. For each construct, the cytotoxicity assay was performed in quadruplicate, except for assay of pAS38 and 39, which were performed in triplicate. The values of LDH release for each construct were compared against either F(ab′)₂ or whole antibody, or each other, using a one-tailed t-test in Excel.

Results

FIGS. 26 and 27 shows that there is negligible cell killing with either pAS6 (HMFG1 alone) or with pAS41 (F(ab′)₂ alone). All of the hHMFG1 F(ab′)₂-DNase I constructs kill significantly more cells than the F(ab′)₂ fragment alone (p<0.00193) and all of the antibody-DNase I constructs kill significantly more cells than antibody alone (p<0.00783), except for perhaps pAS34 (p<0.021).

(F) Use of the DNase-I/huHMFG-1 Fab Fusion Protein in the Treatment of Ovarian Cancer

Patients diagnosed with ovarian cancer are treated by intravenous injection of the DNaseI/huHMFG-1 Fab fusion protein. Typically, a dose of between 1 to 100 mg will be administered weekly.

Therapeutic response is measured by the normal clinical procedures that are well known in the art, for example radio-imaging methods.

### Example 2

(A) Mammalian expression of humanised HMFG-1/DNase constructs

In a second series of experiments, two further humanised HMFG-1/Dnase constructs were expressed in mammalian cells. The first construct encoded a fusion protein a complete hHMFG-1 antibody fused with human DNase, designated ‘Ad-DNase’. The second construct encoded a fusion protein a Fab fragment of the hHMFG-1 antibody fused with human DNase, designated ‘Fab-DNase’. Ad-Dnase and Fab-DNase are shown schematically in FIG. 28.

Ad-DNase comprises an HMFG-1 light chain as shown in FIG. 3(c) and an HMFG-1 heavy chain/DNase fusion as shown in FIG. 7(b).

Fab-DNase comprises an HMFG-1 light chain as shown in FIG. 3(c) and an HMFG-1 Fd chain/DNase fusion as shown in FIG. 14(d).

The human HMFG1 heavy and light chain constructs were cloned into the pEE6 expression vector system for expression in mammalian CHO or myeloid NS0 cells, as described in Section (A) of Example 1. This vector consists of two human cytomegalovirus promoters (hCMV) for both the heavy and light chain genes. Each transcription unit is completed by the poly-adenylation signal (pA) with an optional immunoglobulin terminator sequence (Ig term.) located between the heavy and light chain transcription units. The vectors also comprise a 5′-UT Kozak sequence (to enhance translation of the mRNA) and an ATG initiator codon upstream of both heavy and light chains.

The vectors encoding Ad-Dnase and Fab-DNase, designated pAS34K and pAS 302 respectively, are shown schematically in FIG. 32.

Propagation in E. coli can be selected for by the presence on an ampicillin resistance gene. The inclusion of a glutamine synthetase gene (GS) in the vector allows the stable NS0 transfectomas to be selected by growth in glutamine free media, since NS0 cells are GS⁼ and cannot otherwise grow in glutamine free media.

These plasmids were co-transfected with a vector containing a neomycin resistance gene into CHO cells. Stable cell lines were generated for each of the constructs.

Clones were selected that expressed DNase activity and antigen (PEM)-binding activity.

(B) Purification of hHMFG-1/DNase Constructs

The cells were routinely grown in:

| | |
|---|---|
| DMEM (Gibco 10938-025) | 500 ml |
| Non essential amino acids (Sigma M7145) | 5 ml |
| Sodium pyruvate (Sigma S8636) | 5 ml |
| Glutamine (G7513) | 5 ml |
| Heat inactivated foetal calf serum | 50 ml |

Incubation was carried out at 37° C. in 5% CO₂.

For production of the Ab-DNase fusion protein, W70 cells (CHO cells transfected with pAS34K) were maintained in flats and grown to confluency in T175 flasks. Each T175 flask was split between two 850 cm² roller bottles containing 100 ml of the aforementioned growth media. Each roller bottle was gassed with an 95% air 5% CO₂ mix for 1 minute and then sealed. They were rolled at a rate of 0.5 rpm and were gassed every other day as described earlier until the cultures were confluent. At this stage the medium was removed and 200 ml of harvest medium was replaced on the culture. This was the same medium but contained 2 mM sodium butyrate (with or without 10% heat inactivated FCS). The cells were then grown for a further 3-4 days before they were harvested. The medium was collected from the cells and dead cells were removed from the medium by centrifugation at 5000 rpm for 30 mins at 4° C. The spun medium (supernatant) was then filtered through a 0.2 micron filter unit, prior to applying to the affinity chromatography column.

The Fab-DNase fusion product was then purified by affinity chromatography using a Protein-L column (Protein L agarose, P3351 from Sigma Co, Poole, Dorset, UK), as follows:

1. Wash 1 ml of settled protein L agarose (P3351) with at least 5 volumes of phosphate buffered saline (PBS: 10 mM phosphate buffered saline, pH 7.4).

2. Dilute 1 ml supernatant with 9 ml PBS.

3. Mix diluted supernatant with protein-L agarose and incubate with gentle end over end mixing for 1 hour at room temperature.

4. Pack the slurry in a column and drain.

5. Wash away unbound proteins with 10-15 column volumes of PBS.

6. Elute bound protein with5 ml elution buffer (0.1 M glycine, pH 2.0, or 0.2 M citrate buffer, pH2.8).

7. Neutralise eluted material with Tris-base to achieve pH 7.5.

FIG. 31(a) shows the elution profile of the Fab-DNase from the Protein-L column when eluted with 0.1 M glycine, pH 2.0.

Following purification, Fab-DNase was analysed by analytical size-exclusion chromatography on a Superdex-200 column.

FIG. 31(b) shows the size-exclusion chromatogram obtained for the Fab-DNase.

The Ab-DNase fusion product was purified by affinity chromatography using a Protein-A sepharose column, as follows:

1. 25 ml of protein A sepharose fast flow resin (Amersham Pharmacia Biotech) in an XK26 column (Amersham Pharmacia Biotech) was equilibrated in 0.1M glycine, pH8.8, 0.5M NaCl.

2. Approximately 2 liters of sterile-filtered supernatant from cell line W70 (CHO cell line making 34K) was passed the column overnight at a low flow rate (1-2 ml/min).

3. The column was then washed down to base-line and was re-equilibrated in 0.15M disodium hydrogen phosphate, pH9.0 and the bound 34K was eluted by running a gradient between this buffer (A) and a low pH buffer (B) which consisted of 0.1M citric acid, pH2.0, supplemented to 2 mM calcium chloride and 2 mM magnesium sulphate. The gradient was run over 100 ml at a flow rate of 4 ml/min and a further 50 ml of buffer B was run over the column at the completion of the gradient, also at 4 ml/min.

4. During the 100 ml gradient and the last 50 ml of buffer A fractions were collected. The peak fractions were identified and pooled and dialysed against 4 liters of 25 mM Hepes, pH7.5, 0.2 M NaCl, 1 mM calcium chloride and 1 mM magnesium sulphate. Dialysis was performed overnight at 4C.

5. The dialysate was concentrated on Centricon spin concentrators to a final concentration of 6-13 mg/ml. The concentration was determined by dividing by its extinction coefficient of 1.558 (calculated from the known sequence).

FIG. 32(a) shows the elution profile of the Ab-DNase from the Protein-L column when eluted with a gradient of 0.15 M Na₂HPO₄, pH 9.0 to 0.1 M citric acid, pH 2.0 containing 2 mM each of CaCl₂ and MgCl₂.

FIG. 32(b) shows the size-exclusion chromatogram obtained for the Ab-DNase.

(C) Determination of Concentration of Fusion Proteins

Prior to measuring DNase activity of the purified fusion proteins (see Section (E) below), the concentration of the proteins was determined by ELISA, as follows (see also Section (C) of Example 1).

Materials

1. 96 Well ELISA plates (Nunc F96 Maxisorp Cat No. 442404).

2. Bovine serum albumin (Sigma A-9647).

3. Coating buffer (Na2CO3 1.59 g/l, NaHC03 2.93 g/l, NaN3 0.2 g/l, pH9.6.

4. GST-MUC1-7TR antigen (1.5 mg/ml).

5. Anti-human kappa light-chain antibody GD12 (0.2 mg/ml, Binding Site, MC135).

6. Peroxidase-conjugated rabbit anti-mouse IgG (Jackson, 315-035-045).

7. TMB- substrate buffer (Sigma P-4417).

8. Tween 20 (Sigma P7949).

9. Purified humanised HMFG1 (1.4 mg/ml).

Method

Note all washes in this protocol consist of 3×3 min washes in PBS buffer (note: all PBS buffer contained 0.05% Tween) and the plate was incubated in a lunch box containing moist tissue paper.

1. Coat 100 ng of antigen/100 μl coating buffer/well overnight at 4° C.

2. Wash the plate and block each well with 100 μl of PBS containing 0.05% Tween, and 1% BSA for 1 h at 30° C. Wash plate afterwards.

3. A standard curve of humanised HMFG1 should be prepared down the plate using doubling dilutions. Make each dilution in 100 μl PBS buffer and for the highest concentration in the curve use 1000 ng of antibody.

4. Incubate the plate for 2 h at 30° C, wash, and add 100 μl PBS containing 200 ng of the anti-human Kappa light chain antibody to each well of the plate. Incubate for a further 1 h at 30° C. and then wash the plate.

5. Add 100 μl PBS containing the rabbit anti-mouse IgG-peroxidase conjugate (diluted 1:2000) to each well of the plate and incubate for 30 min at 30° C. Wash the plate and add 100 ∥l TMB- substrate-buffer to each well of the plate and allow the reaction to proceed in the dark at room temperature. When the blue colour has developed, read the plate at a wavelength of 630 nm.

(D) SDS-PAGE

Following purification of Ab-DNase and Fab-DNase, the fusion proteins were analysed by SDS-PAGE under non-reducing and reducing conditions, as described in Section (B) of Example 1.

In brief, affinity-purified material was used. In the case of the Ab-DNase fusion protein, this was from a sample dialysed and concentrated (as described in the protein A protocol above). In the case of the Fab-DNase, this was unconcentrated protein directly eluted from the protein L affinity column. 15 ul of the Fab-DNase protein-L eluate was mixed with 5 ul of either reducing or non-reducing loading buffer whereas 2 ul of the Ab-DNase protein A eluate (dialysed and concentrated) was mixed with 5 ul of either reducing or non-reducing buffer. Both samples were boiled for 5 minutes and were loaded onto the gel. The gels were stained with Coomassie Brilliant Blue stain. The cells were not labelled with 35S-methionine (as in Example 1).

The SDS-PAGE autoradiograph for Ab-DNase is shown in FIG. 33(a). Under reducing conditions, Ab-DNase produces a band of about 80 kDa, which corresponds to the expected size of the heavy chain-DNase fusion product (see lane 3). A further band of about 50 kDa is also observed, which is approximately the same molecular weight as the hHMFG-1 heavy chain (see lane 4).

The SDS-PAGE autoradiograph for Fab-DNase is shown in FIG. 33(b). Under reducing conditions, Fab-DNase produces a band of about 55-60 kDa, which corresponds to the expected size of Fab-DNase (see lane 3). Under non-reducing conditions, a band of about 80-85 kDa is observed, which is the approximate molecular weight of Fab-DNase rather than F(ab′)₂-DNase (see lane 4). Thus, the Fab-DNase appears to exist as a dimer of the hHMFG-1 light chains and the hHMFG-1 heavy chain/human DNase fusion, not a tetrameric F(ab′)₂-DNase.

(E) Measurement of DNase Activity of hHMFG-1/DNase Constructs

DNase activity of the two fusion proteins was determined as described in Section (D) of Example 1. In brief, 0.1 ml of the purified protein was added to a 1:1 solution of assay buffer and methyl green-salmon sperm DNA complex, and the mixture incubated at 37° C. A reduction in absorbance at 620 mn is indicative of DNA activity.

A standard curve produced using bovine DNase I is shown in FIG. 34(a).

FIG. 34(b) shows the DNase activity of the Fab-DNase and Ab-DNase fusion proteins 3 h and 6 h after being added to the DNA, compared to a positive control of bovine DNase and a negative control of Fab only. Clearly, the DNase activity of the Fab-DNase and Ab-DNase fusion proteins is comparable to that of the bovine DNase positive control.

(F) Cytotoxicity of DNase Activity of hHMFG-1/DNase Constructs

Cytotoxocity of the Fab-DNase and Ab-DNase fusion proteins was analysed using two tumour cell lines, the human malignant melanoma cell line A375 and the human ovarian adenocarcinoma cell line OVCAR 3.

An initial cell-based ELISA was performed using hHMFG-1 antibodies to determine the level of expression of PEM (the MUCL gene product) on these cells.

Cell-based PEM ELISA Assay Protocol

Materials and Methods

1. Phosphate buffered saline tablets (Sigma P-4417)

2. 50% glutaraldehyde solution (BDH UN2810 Prod. 2868240)

3. sodium azide (Sigma S-8032)

4. Nunclon 96 well tissue culture plate (Nunc D167008)

5. BSA (Sigma A-9647)

6. OVCAR-3 ovarian cancer cells, A375 melanoma cancer cells both from ATCC

7. TMB substrate buffer (Sigma P-4417)

8. Tween 20 (Sigma P7949)

9. Purified humanised HMFG1 (1 mg/ml from ICRF)

10. RPMI 1640 media (Gibco 21875-034)

Protocol

1. The OVCAR-3 and A375 cells were grown in RPMI containing 20% and 10% FCS respectively at 37° C. in 5% CO2 in a 96 well tissue culture plate, seeded at 106 cells/ml with 0.1 nil/well.

2. Excess media was removed and the plate was fixed with 0.05% glutaraldehyde in water for 1 hour at room temperature.

3. Excess glutaraldehyde/water solution was removed and the plates were washed three times with PBS containing 0.05% Tween 20. The plate was stored at 4° C. until required in PBS with 0.02% sodium azide).

4. To use the plate, the plate was then washed with three washes of PBS containing 0.05% Tween 20, and the wells were blocked with 0.1 ml 5% BSA in PBS containing 0.05% Tween 20. The wells were blocked for 1 hour at 30° C.

5. They washed three times as described before. Serial dilutions of hHMFG1 were plated out on the wells from a maximum concentration of 2 μg/ml downward. Dilutions of constructs were also similarly plated onto the fixed cells. All dilutions were prepared in PBS containing 0.05% Tween 20.

6. The proteins were incubated with the fixed cells for 1 hour at 30° C. and were again washed three times as described above.

7. Anti-human IgG-Fc peroxidase conjugate antibody (Jackson 209-035-103) was diluted to 1:2000 in PBS containing 0.05% Tween 20. This was incubated at 30° C. for 30 minutes.

8. Once again the cells were washed as described as before. Then 0.1 ml TMB substrate was put in each well and the colour was developed at room temperature and the absorbance at 655 nm was determined.

For comparison, an additional ELISA using Ab-DNase was performed with the OVCAR 3 cells.

Antigen-bound hHMFG-1 and Ab-DNase was detected by a peroxidase-conjugated anti-human Fc antibody.

The results of the ELISA are shown in FIG. 35, indicating that the OVCAR 3 cell line expresses high levels of PEM (as measured by both hHMFG-1 and Ab-DNase) while the A375 cell line expresses low levels of PEM (and hence can be used as a negative control in cytotoxicity experiments).

Cytotoxicity was measured using an LDH release assay, as described in Section (E) of Example 1. In brief, 10⁵ cells per well of the A375 and OVCAR 3 cell lines were plated in a 96-well plate and grown for 24 hours. Fifteen microliters of the purified fusion proteins (containing 200 ng of Ab-DNase or 100 ng of Fab-DNase) were added to the cells and incubated for 48 hours at 37° C. A negative control group of each cell type was treated with 200 ng of the hHMFG-1 antibody (i.e. not fused to DNase).

Following the incubation period, 50 μl of the supernatant was removed and incubated with 50 μl of tetrazolium-containing substrate buffer for 30 minutes at 22° C. The reaction was stopped with stop buffer (Promega) and the absorbance of the reaction mixture at 490 nm measured.

Both Fab-DNase and Ab-DNase fusions show cell killing of OVCAR 3 cells as compared to the negative control hHMFG-1 treated cells. In contrast, killing of A375 cells by DNase fusions is negligible, consistent with negligible binding of the fusions to these cells.

1. A compound comprising a target cell-specific portion and a cytotoxic portion characterised in that:
(i) the target cell-specific portion comprises an humanised monoclonal antibody having specificity for polymorphic epithelial mucin (PEM), or an antigen binding fragment thereof; and
(ii) the cytotoxic portion has endonucleolytic activity.

2. A compound according to claim 1 wherein the target cell-specific portion comprises an humanised HMFG-1 antibody or an antigen binding fragment thereof.

3. A compound according to claim 2 wherein the target cell-specific portion is an humanised HMFG-1 antibody.

4. A compound according to claim 1 or 2 wherein the target cell-specific portion comprises an antigen binding fragment of the humanised antibody selected from the group consisting of Fab-like molecules, such as Fab and F(ab′)₂, Fv molecules, disulphide-linked Fv molecules, ScFv molecules and single domain antibodies (dAbs).

5. A compound according to claim 4 wherein the target cell-specific portion comprises a Fab molecule.

6. A compound according to claim 4 wherein the target cell-specific portion comprises a F(ab′)₂ molecule.

7. A compound according to claim 1 wherein the target cell-specific portion comprises an amino acid sequence encoded by at least part of one or both of the nucleotide sequences of FIG. 3(a) and (d).

8. A compound according to claim 7 wherein the target cell-specific portion comprises an amino acid sequence encoded by the nucleotide sequence of FIG. 3(a) and an amino acid sequence encoded by the nucleotide sequence of FIG. 3(d).

9. A compound according to any one of claims 1 to 8 wherein the cytotoxic portion has DNA endonucleolytic activity.

10. A compound according to claim 9 wherein the cytotoxic portion is at least the catalytically active portion of a DNA endonuclease.

11. A compound according to claim 10 wherein the endonuclease is a mammalian deoxyribonuclease I.

12. A compound according to claim 11 wherein the endonuclease is a human deoxyribonuclease I.

13. A compound according to claim 1 wherein the endonuclease is a restriction endonuclease.

14. A compound according to claim 10 wherein the cytotoxic portion comprises the amino acid sequence shown in FIG. 2(a) or (b).

15. A compound according to any one of claims 1 to 14 wherein a nuclear localization signal is incorporated.

16. A compound according to claim 15 wherein the nuclear localization signal comprises the sequence PKKKRKV.

17. A compound according to any one of claims 1 to 16 wherein the target cell-specific portion and the cytotoxic portion are fused.

18. A compound according to claim 17 wherein the target cell-specific portion and the cytotoxic portion are separated by a linker sequence.

19. A compound according to claim 18 wherein the linker sequence is or comprises GG or GSGG.

20. A compound according to any one of claims 1 to 19 wherein the compound comprises all or part of the amino acid sequence as shown in FIG. 3(c) together with all or part of an amino acid sequence selected from the group consisting of amino acid sequences as shown in FIGS. 5(d), 6(d), 7(b), 8(b), 9(b), 10(b), 11(b), 12(b), 13(d), 14(d), 15(d), 16(c), 17(d), 18(d) and 19(d).

21. A compound according to claim 20 wherein the compound comprises an amino acid sequence as shown in FIG. 3(c) and an amino acid sequence as shown in FIG. 7(b).

22. A compound according to claim 20 wherein the compound comprises an amino acid sequence as shown in FIG. 3(c) and an amino acid sequence as shown in FIG. 14(d).

23. A nucleic acid molecule encoding a compound as defined in any one of claims 1 to 22.

24. A nucleic acid molecule according to claim 23 wherein the molecule comprises all or part of the nucleotide sequence as shown in FIG. 3(a or b) together with all or part of a nucleotide sequence selected from the group consisting of nucleotide sequences as shown in FIGS. 5(a, b and c), 6(a, b and c), 7(a), 8(a), 9(a), 10(a), 11(a), 12(a), 13(a, b and c), 14(a, b and c), 15(a, b and c), 16(a and b), 17(a, b and c), 18(a, b and c) and 19(a, b and c).

25. A nucleic acid molecule according to claim 24 wherein the molecule comprises a nucleotide sequence as shown in FIG. 3(b) and a nucleotide sequence as shown in FIG. 7(a).

25. A nucleic acid molecule according to claim 24 wherein the molecule comprises a nucleotide sequence as shown in FIG. 3(b) and a nucleotide sequence as shown in FIG. 14(c).

26. A nucleic acid molecule according to any one of claims 23 to 25 wherein the molecule further comprises a Kozak consensus ribosome-binding site.

27. A vector comprising a nucleic acid molecule according to any one of claims 23 to 26.

28. A host cell comprising a vector according to claim 27.

29. A pharmaceutical composition comprising a compound according to any one of claims 1 to 22 and a pharmaceutically acceptable carrier.

30. A compound according to any one of claims 1 to 22 for use in medicine.

31. Use of a compound according to any one of claims 1 to 22 in the preparation of a medicament for treating a mammal having said target cells to be destroyed.

32. A method of treating a mammal having target cells to be destroyed, the method comprising administering a compound according to any one of claims 1 to 22 to said mammal.

33. A use according to claim 31 or a method according to claim 32 wherein the mammal is a human.

34. A use according to claim 31 or a method according to claim 32 wherein the target cells to be destroyed are cancer cells.

35. A use or a method according to claim 34 wherein the cancer cells are epithelial cancer cells.

36. A use or a method according to claim 35 wherein the cancer cells are ovarian, gastric, colorectal and/or pancreatic cancer cells.
